(19) United States
(12) Patent Application Publication
(10) Pub. No.: US 2026/0263598 A1
(43) Pub. Date: Sep. 10, 2026

(54) CHIMERIC ANTIGEN RECEPTORS AND CELL THERAPIES FOR AUTOIMMUNE DISEASES

(71) Applicant: Kite Pharma, Inc., Santa Monica, CA (US)

(72) Inventors: Ruben Alvarez Rodriguez, West Hills, CA (US); Andre S.S.H. Jung, Los Angeles, CA (US); Jodi L. Murakami, Culver City, CA (US); Myrna R. Nahas, Cypress, TX (US); Arianne C. Perez-Garcia, Woodland Hills, CA (US); Stuart A. Sievers, Van Nuys, CA (US); Jed J.W. Wiltzius, Winchester, MA (US)

(21) Appl. No.: 19/560,901

(22) Filed: Mar. 9, 2026

Related U.S. Application Data
(60) Provisional application No. 63/769,596, filed on Mar. 10, 2025.
(60) Provisional application No. 63/789,143, filed on Apr. 15, 2025.
(60) Provisional application No. 63/913,625, filed on Nov. 7, 2025.

(51) Int. Cl. A61K 40/41; A61K 40/11; A61K 40/31; A61P 37/06; C07K 14/705; C07K 16/28

(52) U.S. Cl. CPC A61K 40/41; A61K 40/11; A61K 40/31; A61P 37/06; C07K 14/7051; C07K 16/2803; C07K 16/2887

(57) ABSTRACT

Provided herein are new anti-CD20 chimeric antigen receptors (CARs) which, alone with together with an anti-CD19 CAR, can be suitably used for treating diseases or conditions mediated by dysregulated B cells, such as cancers and autoimmune diseases.

### CROSS REFERENCE TO RELATED APPLICATIONS

This application claims the benefit under 35 U.S.C. § 119(e) of the U.S. Provisional Application Ser. No. 63/769,596, filed Mar. 10, 2025, No. 63/789,143, filed Apr. 15, 2025, and No. 63/913,625, filed Nov. 7, 2025, the content of each of which is hereby incorporated by reference in its entirety.

### REFERENCE TO AN ELECTRONIC SEQUENCE LISTING

The content of the electronic sequence listing (407570.xml; Size: 41,737 bytes; and Date of Creation: Feb. 5, 2026) is herein incorporated by reference in its entirety.

### BACKGROUND

Human CD19 is a 95 kDa transmembrane protein expressed only in the B-cell lineage. CD19 is a signal transduction molecule that plays an important role in establishing B-cell signaling thresholds required to regulate the development, activation, and differentiation of B cells. CD19 is expressed in all normal B cells starting at the pre-B-cell stage until the final differentiation stage and is not expressed in pluripotent hematopoietic stem cells (HSCs), granulocytes, monocytes, platelets, erythrocytes, T lymphocytes, or most plasma cells.

Human CD20 is a 33 kDa surface antigen expressed by B-cells with levels varying among different subtypes and across patients within a subtype. Expression of CD20 is restricted to B cells from the pre-B-cell stage to the memory B-cell stage, plasmablasts, and stimulated plasma cells; this offers a targeting opportunity with an off-target toxicity profile similar to CD19.

The pattern of CD19 and CD20 expression is maintained in most B-cell malignancies. In autoimmune diseases, aberrant B-cell reactivity to normal constituents of the host play an important role in the etiology and pathogenesis of these conditions. Besides production of pathogenic autoantibodies, B cells activate T cells and release inflammatory and immuno-modulatory cytokines and chemokines, which promote local inflammation and fibrosis.

Given the prominent role of dysregulated B cells in autoimmune diseases, inhibition of B cells has been one of the main foci of treatment of autoimmune diseases for decades. The broad applicability of this approach is supported by the Food and Drug Administration (FDA)-approved B-cell-directed therapies over the past two decades in conditions like rheumatoid arthritis (RA), antineutrophil cytoplasmic antibody (ANCA)-associated vasculitis, SLE, multiple sclerosis (MS), neuromyelitis optica, and pemphigus vulgaris. The targeting of CD19, CD20, or B-lymphocyte stimulator/B-cell activating factor (BLyS/BAFF) have all shown encouraging results; however, mortality and morbidity in these diseases remain high, particularly with severe manifestations like nephritis and interstitial lung disease (ILD). One hypothesis for relapsing after CD20-targeting therapy in SLE/LN is the potential for incomplete B-cell depletion, which results in the persistence of pathogenic B cells in the lymph nodes, kidneys, and other organs. With anti-CD20-directed therapies, such as rituximab, mature CD20⁺ B cells are depleted, but not CD19⁺CD20⁻ B cells, including the majority of pathogenic plasma blasts, which are abnormally increased in patients with SLE and correlate with disease activity and severity. Limited tissue penetration and the dependence on additional effector cells such as macrophages and natural killer cells may pose additional limiting factors for the effectiveness of rituximab (and other antibody-based therapies) in SLE. Furthermore, the long-term treatment burden of all the currently approved therapies presents significant challenges in patients living with chronic autoimmune diseases.

Emerging early data on B-cell depletion using novel CAR T-cell therapies in various autoimmune diseases highlight the potential for reestablishing immune tolerance and achieving rapid, sustained remission without chronic use of systemic immunosuppressants. Achieving durable remission without chronic systemic immunosuppression is a highly desired outcome in the treatment of autoimmune diseases.

Targeting CD19 via CAR T cells has been shown to provide more comprehensive B-cell depletion compared with antibody-mediated targeting of CD20 alone. Expression of CD19 in B cells starts prior to the expression of CD20 at the late pro-B-cell stage and is maintained until plasma cell differentiation, which is then associated with partial loss of CD19 expression. Therefore, anti-CD19 therapy can potentially target autoantibody-secreting plasma blasts and plasma cells as well as early B-cell differentiation stages not covered by anti-CD20 therapy.

Targeting both CD20 and CD19 may provide more comprehensive B-cell depletion than what could be achieved by targeting either antigen alone. The cell surface density of CD19 is lower than that of CD20 in normal B cells, and variability in CD19 expression may render CD19 single-targeting inadequate to achieve optimal B-cell depletion, both in circulation and in tissues of patients with autoimmune diseases.

### SUMMARY

Provided herein are new anti-CD20 chimeric antigen receptors (CARs) which, alone with together with an anti-CD19 CAR, can be suitably used for treating diseases or conditions mediated by dysregulated B cells, such as cancers and autoimmune diseases.

One embodiment of the present disclosure provides a method for treating an autoimmune disease or condition in a patient in needed thereof, comprising administering to the patient a T cell comprising an anti-CD19 chimeric antigen receptor (CAR) that specifically binds CD19 and an anti-CD20 CAR that specifically binds CD20, wherein the anti-CD19 CAR comprises a first antigen binding fragment comprising a heavy chain variable region (VH) comprising complementarity determining regions (CDR) 1, 2, and 3, and a light chain variable region (VL) comprising a CDR1, a CDR2 and a CDR3, wherein the VH CDR1, VH CDR2, VH CDR3, VL CDR1, VL CDR2 and VL CDR3 of the anti-CD19 CAR comprise the amino acid sequences of SEQ ID NO:21-26, respectively, and the anti-CD20 CAR comprises a second antigen binding fragment comprising a VH comprising a CDR1, a CDR2, and a CDR3, and a VL comprising a CDR1, a CDR2 and a CDR3, wherein the VH CDR1, VH CDR2, VH CDR3, VL CDR1, VL CDR2 and VL CDR3 of the anti-CD20 CAR comprise the amino acid sequences of SEQ ID NO:27-32, respectively.

In some embodiments, the anti-CD19 CAR further comprises a first transmembrane domain and/or a first CD3 zeta signaling domain. In some embodiments, the anti-CD20 CAR further comprises a second transmembrane domain and/or a first CD3 zeta signaling domain.

In some embodiments, the anti-CD19 CAR further comprises a first co-stimulatory domain, and the anti-CD20 CAR further comprises a second co-stimulatory domain, wherein the first co-stimulatory domain comprises a CD28 intracellular signaling domain and the second co-stimulatory domain comprises a 4-1BB intracellular signaling domain.

In some embodiments, the CD28 intracellular signaling domain comprises an amino acid sequence that is at least 90% identical to SEQ ID NO:10. In some embodiments, the 4-1BB intracellular signaling domain comprises an amino acid sequence that is at least 90% identical to SEQ ID NO:17.

In some embodiments, the autoimmune disease or condition is a rheumatological autoimmune disease or condition, or a neurological autoimmune disease or condition.

In some embodiments, the rheumatological autoimmune disease or condition is selected from the group consisting of lupus nephritis (LN), non-renal systemic lupus erythematosus (SLE), systemic sclerosis (SSc), idiopathic inflammatory myopathies (IIM), Anti-neutrophil cytoplasmic antibody-associated (ANCA) vasculitis, Sjogren's syndrome, rheumatoid arthritis (RA), and pediatric lupus.

In some embodiments, the neurological autoimmune disease or condition is selected from the group consisting of myasthenia gravis, multiple sclerosis, and chronic inflammatory demyelinating polyneuropathy.

In some embodiments, the autoimmune disease or condition is relapsed/refractory or moderate to severe, progressive systemic lupus erythematosus (SLE) with or without lupus nephritis (LN), systemic sclerosis (SSc), or idiopathic inflammatory myopathy (IIM).

In some embodiments, the T cell is administered to the patient at a dose of 0.2×10⁶ to 2×10⁶ cells per kilogram of body weight of the patient, but not greater than 1×10⁸ cells. In some embodiments, the T cell is administered to the patient at a dose of 0.4×10⁶ to 0.6×10⁶ cells per kilogram of body weight of the patient. In some embodiments, the T cell is administered to the patient at a dose of 0.8×10⁶ to 1.2×10⁶ cells per kilogram of body weight of the patient.

In some embodiments, the T cell is administered no more than once every 4 weeks, 1 month, 2 months, 3 months, 6 months, 1 year, 2 year, or 5 years.

In some embodiments, the T cell is autologous or allogeneic.

In some embodiments, the first antigen binding fragment or the second antigen binding fragment is a single chain fragment (scFv). In some embodiments, the VH and VL of the anti-CD19 CAR comprise the amino acid sequences of SEQ ID NO:4 and 5, respectively. In some embodiments, the anti-CD19 scFv comprises an amino acid sequence that is at least 90% identical to SEQ ID NO:7. In some embodiments, the VH and VL of the anti-CD20 CAR comprise the amino acid sequences of SEQ ID NO:12 and 13, respectively. In some embodiments, the anti-CD19 scFv comprises an amino acid sequence that is at least 90% identical to SEQ ID NO:14.

In some embodiments, the first transmembrane domain and the second transmembrane domain each, independently, comprises a transmembrane domain of a protein selected from the group consisting of CD28, CD8alpha, CD8beta, 4-1BB, B7-H3, BAFFR, BLAME, BTLA, CD100, CD103, CD11a, CD11b, CD11c, CD11d, CD160, CD18, CD19, CD19a, CD2, CD247, CD27, CD276, CD29, CD3 delta, CD3 epsilon, CD3 gamma, CD30, CD4, CD40, CD49a, CD49D, CD49f, CD69, CD7, CD79a, CD84, CD96, CDS, CEACAM1, CRT AM, DAP-10, DNAM1, Fc gamma receptor, GADS, GITR, HVEM, IA4, ICAM-1, ICOS, IL-2R beta, IL-2R gamma, IL-7R alpha, ITGA4, ITGA6, ITGAD, ITGAE, ITGAL, ITGAM, ITGAX, ITGB1, ITGB2, ITGB7, KIRDS2, LAT, LFA-1, LIGHT, LTBR, Ly9, NKG2C, NKG2D, NKp30, NKp44, NKp46, NKp80, OX-40, PAG, PD-1, PSGL1, SELPLG, SLAM, SLAMF4, SLAMF6, SLAMF7, SLP-76, TNFR2, TNFSFi4, TRANCE, VLA1, VLA-6, a cytokine receptor, a MHC class 1 molecule, a SLAM protein, and a TNF receptor protein. In some embodiments, the first transmembrane domain comprises the amino acid sequence of SEQ ID NO:9. In some embodiments, the second transmembrane domain comprises the amino acid sequence of SEQ ID NO:16.

In some embodiments, the anti-CD19 CAR further comprises a first hinge between the anti-CD19 antigen binding fragment and the first transmembrane domain, and the anti-CD20 CAR further comprises a second hinge between the anti-CD20 antigen binding fragment and the second transmembrane domain. In some embodiments, the first hinge and the second hinge each, independently, comprises a hinge region of an IgG, CD28 or CD8alpha. In some embodiments, the first hinge comprises the amino acid sequence of SEQ ID NO:8. In some embodiments, the second hinge comprises the amino acid sequence of SEQ ID NO:15.

In some embodiments, the anti-CD19 CAR comprises an amino acid sequence that is at least 90% identical to SEQ ID NO:1. In some embodiments, the anti-CD20 CAR comprises an amino acid sequence that is at least 90% identical to SEQ ID NO:2. In some embodiments, the anti-CD19 CAR comprises the amino acid sequence of SEQ ID NO:1 and the anti-CD20 CAR comprises the amino acid sequence of SEQ ID NO:2.

In some embodiments, the T cell comprises a polynucleotide comprising an open reading frame (ORF) that encodes a bicistronic CAR that comprises the anti-CD19 CAR and the anti-CD20 CAR. In some embodiments, the bicistronic CAR comprises a cleavable linker between the anti-CD19 CAR and the anti-CD20 CAR. In some embodiments, the cleavable linker comprises a furin recognition site and/or a ribosomal skipping signal. In some embodiments, the cleavable linker comprises an amino acid sequence that is at least 90% identical to SEQ ID NO:19.

In some embodiments, the bicistronic CAR comprises an amino acid sequence that is at least 90% identical to SEQ ID NO:3. In some embodiments, the bicistronic CAR comprises the amino acid sequence of SEQ ID NO:3. In some embodiments, the polynucleotide comprises a nucleic acid sequence that is at least 90%, 95 or 100% identical to SEQ ID NO:39. In some embodiments, the polynucleotide comprises the nucleic acid sequence of SEQ ID NO:39.

Also provided, in one embodiment, is a T cell comprising an anti-CD19 chimeric antigen receptor (CAR) that specifically binds CD19 and an anti-CD20 CAR that specifically binds CD20, wherein the anti-CD19 CAR comprises a first antigen binding fragment comprising a heavy chain variable region (VH) comprising complementarity determining regions (CDR) 1, 2, and 3, and a light chain variable region (VL) comprising a CDR1, a CDR2 and a CDR3, wherein the VH CDR1, VH CDR2, VH CDR3, VL CDR1, VL CDR2 and VL CDR3 of the anti-CD19 CAR comprise the amino acid sequences of SEQ ID NO:21-26, respectively, and the anti-CD20 CAR comprises a second antigen binding fragment comprising a VH comprising a CDR1, a CDR2, and a CDR3, and a VL comprising a CDR1, a CDR2 and a CDR3, wherein the VH CDR1, VH CDR2, VH CDR3, VL CDR1, VL CDR2 and VL CDR3 of the anti-CD20 CAR comprise the amino acid sequences of SEQ ID NO:27-32, respectively.

In some embodiments, the anti-CD19 CAR further comprises a first transmembrane domain and/or a first CD3 zeta signaling domain. In some embodiments, the anti-CD20 CAR further comprises a second transmembrane domain and/or a first CD3 zeta signaling domain.

In some embodiments, the anti-CD19 CAR further comprises a first co-stimulatory domain, and the anti-CD20 CAR further comprises a second co-stimulatory domain, wherein the first co-stimulatory domain comprises a CD28 intracellular signaling domain and the second co-stimulatory domain comprises a 4-1BB intracellular signaling domain. In some embodiments, the CD28 intracellular signaling domain comprises an amino acid sequence that is at least 90% identical to SEQ ID NO:10. In some embodiments, the 4-1BB intracellular signaling domain comprises an amino acid sequence that is at least 90% identical to SEQ ID NO:17.

In some embodiments, the T cell is autologous or allogeneic.

In some embodiments, the first antigen binding fragment or the second antigen binding fragment is a single chain fragment (scFv). In some embodiments, the VH and VL of the anti-CD19 CAR comprise the amino acid sequences of SEQ ID NO:4 and 5, respectively. In some embodiments, the anti-CD19 scFv comprises an amino acid sequence that is at least 90% identical to SEQ ID NO:7. In some embodiments, the VH and VL of the anti-CD20 CAR comprise the amino acid sequences of SEQ ID NO:12 and 13, respectively. In some embodiments, the anti-CD20 scFv comprises an amino acid sequence that is at least 90% identical to SEQ ID NO:14.

In some embodiments, the first transmembrane domain and the second transmembrane domain each, independently, comprises a transmembrane domain of a protein selected from the group consisting of CD28, CD8alpha, CD8beta, 4-1BB, B7-H3, BAFFR, BLAME, BTLA, CD100, CD103, CD11a, CD11b, CD11c, CD11d, CD160, CD18, CD19, CD19a, CD2, CD247, CD27, CD276, CD29, CD3 delta, CD3 epsilon, CD3 gamma, CD30, CD4, CD40, CD49a, CD49D, CD49f, CD69, CD7, CD79a, CD84, CD96, CDS, CEACAM1, CRT AM, DAP-10, DNAM1, Fc gamma receptor, GADS, GITR, HVEM, IA4, ICAM-1, ICOS, IL-2R beta, IL-2R gamma, IL-7R alpha, ITGA4, ITGA6, ITGAD, ITGAE, ITGAL, ITGAM, ITGAX, ITGB1, ITGB2, ITGB7, KIRDS2, LAT, LFA-1, LIGHT, LTBR, Ly9, NKG2C, NKG2D, NKp30, NKp44, NKp46, NKp80, OX-40, PAG, PD-1, PSGL1, SELPLG, SLAM, SLAMF4, SLAMF6, SLAMF7, SLP-76, TNFR2, TNFSFi4, TRANCE, VLA1, VLA-6, a cytokine receptor, a MHC class 1 molecule, a SLAM protein, and a TNF receptor protein. In some embodiments, the first transmembrane domain comprises the amino acid sequence of SEQ ID NO:9. In some embodiments, the second transmembrane domain comprises the amino acid sequence of SEQ ID NO:16.

In some embodiments, the anti-CD19 CAR further comprises a first hinge between the anti-CD19 antigen binding fragment and the first transmembrane domain, and the anti-CD20 CAR further comprises a second hinge between the anti-CD20 antigen binding fragment and the second transmembrane domain. In some embodiments, the first hinge and the second hinge each, independently, comprises a hinge region of an IgG, CD28 or CD8alpha. In some embodiments, the first hinge comprises the amino acid sequence of SEQ ID NO:8. In some embodiments, the second hinge comprises the amino acid sequence of SEQ ID NO:15.

In some embodiments, the anti-CD19 CAR comprises an amino acid sequence that is at least 90% identical to SEQ ID NO:1. In some embodiments, the anti-CD20 CAR comprises an amino acid sequence that is at least 90% identical to SEQ ID NO:2. In some embodiments, the anti-CD19 CAR comprises the amino acid sequence of SEQ ID NO:1 and the anti-CD20 CAR comprises the amino acid sequence of SEQ ID NO:2.

In some embodiments, the T cell comprises a polynucleotide comprising an open reading frame (ORF) that encodes a bicistronic CAR that comprises the anti-CD19 CAR and the anti-CD20 CAR. In some embodiments, the bicistronic CAR comprises a cleavable linker between the anti-CD19 CAR and the anti-CD20 CAR. In some embodiments, the cleavable linker comprises a furin recognition site and/or a ribosomal skipping signal. In some embodiments, the cleavable linker comprises an amino acid sequence that is at least 90% identical to SEQ ID NO:19.

In some embodiments, the bicistronic CAR comprises an amino acid sequence that is at least 90% identical to SEQ ID NO:3. In some embodiments, the bicistronic CAR comprises the amino acid sequence of SEQ ID NO:3. In some embodiments, the polynucleotide comprises a nucleic acid sequence that is at least 90%, 95 or 100% identical to SEQ ID NO:39. In some embodiments, the polynucleotide comprises the nucleic acid sequence of SEQ ID NO:39.

In some embodiments, the T cell is administered to a patient to treat autoimmune disease. In some embodiments, the T cell is administered at a dose of about 0.2×10⁶ to about 2×10⁶ cells per kilogram of body weight of the patient, but not greater than about 1×10⁸ cells. In some embodiments, the T cell is administered to the patient at a dose of about 0.4×10⁶ to about 0.6×10⁶ cells per kilogram of body weight of the patient. In some embodiments, the T cell is administered to the patient at a dose of about 0.8×10⁶ to about 1.2×10⁶ cells per kilogram of body weight of the patient.

Yet another embodiment provides a polynucleotide encoding an anti-CD19 chimeric antigen receptor (CAR) that specifically binds CD19 and an anti-CD20 CAR that specifically binds CD20, wherein the anti-CD19 CAR comprises a first antigen binding fragment comprising a heavy chain variable region (VH) comprising complementarity determining regions (CDR) 1, 2, and 3, and a light chain variable region (VL) comprising a CDR1, a CDR2 and a CDR3, wherein the VH CDR1, VH CDR2, VH CDR3, VL CDR1, VL CDR2 and VL CDR3 of the anti-CD19 CAR comprise the amino acid sequences of SEQ ID NO:21-26, respectively, and the anti-CD20 CAR comprises a second antigen binding fragment comprising a VH comprising a CDR1, a CDR2, and a CDR3, and a VL comprising a CDR1, a CDR2 and a CDR3, wherein the VH CDR1, VH CDR2, VH CDR3, VL CDR1, VL CDR2 and VL CDR3 of the anti-CD20 CAR comprise the amino acid sequences of SEQ ID NO:27-32, respectively.

In some embodiments, the anti-CD19 CAR further comprises a first transmembrane domain and/or a first CD3 zeta signaling domain. In some embodiments, the anti-CD20 CAR further comprises a second transmembrane domain and/or a first CD3 zeta signaling domain.

In some embodiments, the anti-CD19 CAR further comprises a first co-stimulatory domain, and the anti-CD20 CAR further comprises a second co-stimulatory domain, wherein the first co-stimulatory domain comprises a CD28 intracellular signaling domain and the second co-stimulatory domain comprises a 4-1BB intracellular signaling domain. In some embodiments, the CD28 intracellular signaling domain comprises an amino acid sequence that is at least 90% identical to SEQ ID NO:10. In some embodiments, the 4-1BB intracellular signaling domain comprises an amino acid sequence that is at least 90% identical to SEQ ID NO:17.

In some embodiments, the first antigen binding fragment or the second antigen binding fragment is a single chain fragment (scFv). In some embodiments, the VH and VL of the anti-CD19 CAR comprise the amino acid sequences of SEQ ID NO:4 and 5, respectively. In some embodiments, the anti-CD19 scFv comprises an amino acid sequence that is at least 90% identical to SEQ ID NO:7. In some embodiments, the VH and VL of the anti-CD20 CAR comprise the amino acid sequences of SEQ ID NO:12 and 13, respectively. In some embodiments, the anti-CD20 scFv comprises an amino acid sequence that is at least 90% identical to SEQ ID NO:14.

In some embodiments, the first transmembrane domain and the second transmembrane domain each, independently, comprises a transmembrane domain of a protein selected from the group consisting of CD28, CD8alpha, CD8beta, 4-1BB, B7-H3, BAFFR, BLAME, BTLA, CD100, CD103, CD11a, CD11b, CD11c, CD11d, CD160, CD18, CD19, CD19a, CD2, CD247, CD27, CD276, CD29, CD3 delta, CD3 epsilon, CD3 gamma, CD30, CD4, CD40, CD49a, CD49D, CD49f, CD69, CD7, CD79a, CD84, CD96, CDS, CEACAM1, CRT AM, DAP-10, DNAM1, Fc gamma receptor, GADS, GITR, HVEM, IA4, ICAM-1, ICOS, IL-2R beta, IL-2R gamma, IL-7R alpha, ITGA4, ITGA6, ITGAD, ITGAE, ITGAL, ITGAM, ITGAX, ITGB1, ITGB2, ITGB7, KIRDS2, LAT, LFA-1, LIGHT, LTBR, Ly9, NKG2C, NKG2D, NKp30, NKp44, NKp46, NKp80, OX-40, PAG, PD-1, PSGL1, SELPLG, SLAM, SLAMF4, SLAMF6, SLAMF7, SLP-76, TNFR2, TNFSF14, TRANCE, VLA1, VLA-6, a cytokine receptor, a MHC class 1 molecule, a SLAM protein, and a TNF receptor protein. In some embodiments, the first transmembrane domain comprises the amino acid sequence of SEQ ID NO:9. In some embodiments, the second transmembrane domain comprises the amino acid sequence of SEQ ID NO:16.

In some embodiments, the anti-CD19 CAR further comprises a first hinge between the anti-CD19 antigen binding fragment and the first transmembrane domain, and the anti-CD20 CAR further comprises a second hinge between the anti-CD20 antigen binding fragment and the second transmembrane domain. In some embodiments, the first hinge and the second hinge each, independently, comprises a hinge region of an IgG, CD28 or CD8alpha. In some embodiments, the first hinge comprises the amino acid sequence of SEQ ID NO:8. In some embodiments, the second hinge comprises the amino acid sequence of SEQ ID NO:15.

In some embodiments, the anti-CD19 CAR comprises an amino acid sequence that is at least 90% identical to SEQ ID NO:1. In some embodiments, the anti-CD20 CAR comprises an amino acid sequence that is at least 90% identical to SEQ ID NO:2. In some embodiments, the anti-CD19 CAR comprises the amino acid sequence of SEQ ID NO:1 and the anti-CD20 CAR comprises the amino acid sequence of SEQ ID NO:2.

In some embodiments, the polynucleotide comprises an open reading frame (ORF) that encodes a bicistronic CAR that comprises the anti-CD19 CAR and the anti-CD20 CAR. In some embodiments, the bicistronic CAR comprises a cleavable linker between the anti-CD19 CAR and the anti-CD20 CAR. In some embodiments, the cleavable linker comprises a furin recognition site and/or a ribosomal skipping signal. In some embodiments, the cleavable linker comprises an amino acid sequence that is at least 90% identical to SEQ ID NO:19.

In some embodiments, the bicistronic CAR comprises an amino acid sequence that is at least 90% identical to SEQ ID NO:3. In some embodiments, the bicistronic CAR comprises the amino acid sequence of SEQ ID NO:3. In some embodiments, the polynucleotide comprises a nucleic acid sequence that is at least 90%, 95 or 100% identical to SEQ ID NO:39. In some embodiments, the polynucleotide comprises the nucleic acid sequence of SEQ ID NO:39.

Another embodiment provides an anti-CD20 chimeric antigen receptor (CAR) that specifically binds CD20, comprising an antigen binding fragment comprising a VH comprising a CDR1, a CDR2, and a CDR3, and a VL comprising a CDR1, a CDR2 and a CDR3, wherein the VH CDR1, VH CDR2, VH CDR3, VL CDR1, VL CDR2 and VL CDR3 of comprise the amino acid sequences of SEQ ID NO:27-32, respectively.

In some embodiments, the anti-CD20 CAR further comprises a transmembrane domain and/or a first CD3 zeta signaling domain. In some embodiments, the anti-CD20 CAR further comprises a co-stimulatory domain. In some embodiments, the co-stimulatory domain comprises an amino acid sequence that is at least 90% identical to SEQ ID NO:17.

In some embodiments, the antigen binding fragment is a single chain fragment (scFv). In some embodiments, the VH and VL of comprise the amino acid sequences of SEQ ID NO:12 and 13, respectively. In some embodiments, the scFv comprises an amino acid sequence that is at least 90% identical to SEQ ID NO:14.

In some embodiments, the transmembrane domain comprises a transmembrane domain of a protein selected from the group consisting of CD28, CD8alpha, CD8beta, 4-1BB, B7-H3, BAFFR, BLAME, BTLA, CD100, CD103, CD11a, CD11b, CD11c, CD11d, CD160, CD18, CD19, CD19a, CD2, CD247, CD27, CD276, CD29, CD3 delta, CD3 epsilon, CD3 gamma, CD30, CD4, CD40, CD49a, CD49D, CD49f, CD69, CD7, CD79a, CD84, CD96, CDS, CEACAM1, CRT AM, DAP-10, DNAM1, Fc gamma receptor, GADS, GITR, HVEM, IA4, ICAM-1, ICOS, IL-2R beta, IL-2R gamma, IL-7R alpha, ITGA4, ITGA6, ITGAD, ITGAE, ITGAL, ITGAM, ITGAX, ITGB1, ITGB2, ITGB7, KIRDS2, LAT, LFA-1, LIGHT, LTBR, Ly9, NKG2C, NKG2D, NKp30, NKp44, NKp46, NKp80, OX-40, PAG, PD-1, PSGL1, SELPLG, SLAM, SLAMF4, SLAMF6, SLAMF7, SLP-76, TNFR2, TNFSFi4, TRANCE, VLA1, VLA-6, a cytokine receptor, a MHC class 1 molecule, a SLAM protein, and a TNF receptor protein. In some embodiments, the transmembrane domain comprises the amino acid sequence of SEQ ID NO:16.

In some embodiments, the anti-CD20 CAR further comprises a hinge between the antigen binding fragment and the second transmembrane domain. In some embodiments, the hinge comprises a hinge region of an IgG, CD28 or CD8alpha. In some embodiments, the hinge comprises the amino acid sequence of SEQ ID NO:15.

In some embodiments, the anti-CD20 CAR comprises an amino acid sequence that is at least 90% identical to SEQ ID NO:2. In some embodiments, the anti-CD20 CAR comprises the amino acid sequence of SEQ ID NO:2.

### BRIEF DESCRIPTION OF THE DRAWINGS

FIG. 1A-B show the cytotoxicity of anti-CD20 CAR containing the Ab4 binder against cells overexpressing CD20, or variants of MUSK or THOP1, on day 1 (A) or day 4 (B).

FIG. 2A-E show B-cell subset frequencies (FRQ) in autoimmune conditions, healthy donors (HDs), and patients With LBCL. Abbreviations: DN, double negative; FRQ, Frequency (%); HD, healthy donor; IIM, Idiopathic inflammatory myopathy; LBCL, large B cell lymphoma; LN, systemic lupus erythematosus with lupus nephritis; SLE, systemic lupus erythematosus without lupus nephritis; SSc, systemic sclerosis. The following B cell subsets were identified by flow cytometry as a percentage (denoted here as FRQ) of total B cells (CD45⁺Lineage-CD19⁺ and/or CD20⁺) from donors with LBCL (n=5), LN (n=7), SLE (n=7) HD (n=14), SSc (n=6) or myositis (n=3): (A) Naive B cells (IgD⁺CD27⁻CD381^{low/-}CD24^{low/-}) (B) Memory B cells (CD27⁺) (C) Memory Switched B cells (IgD⁻CD27⁺IgM⁻) (D) DN B cells (IgD⁻CD27⁻) (E) Plasmablasts (CD45⁺CD19⁺ and/or CD20⁺IgD-CD27⁺CD24⁻CD38^{hi}CD138⁻). Biological replicates are shown as individual datapoints; horizontal bar represents the median value for each group. * indicates statistical significance compared to HD, where at least 3 biological replicates were available. *p<0.05 threshold for significance.

FIG. 3A-F show expression levels of CD19 on B Cells and B-cell subsets from patients with autoimmune conditions, HDs, or patients with LBCL. A: total B cells; B: naive B cells; C: memory B cells; D: memory switched; E: DN B cells; F: plamablasts.

FIG. 4A-F show expression levels of CD20 on B Cells and B-cell subsets from patients with autoimmune conditions, HDs, or patients with LBCL. In FIG. 3-4, CD19 and CD20 expression levels for B-cell subsets identified with surface markers by flow cytometry were quantified using Quantum Simply Cellular beads to calculate ABC as a readout of CD19 and CD20 expression from donors with LBCL (n=5), donors with LN (n=7), healthy donors (n=14), donors with SSc (n=3) or SLE (n=7): (A) Total B cells (CD45⁺Lineage⁻CD19⁺ and/or CD20⁺), (B) Naive B cells (IgD⁺CD27⁻CD38^{low/-}CD24^{low/-}) (C) Memory B cells (IgD⁻CD27⁺) (D) Memory Switched B cells (IgD⁻CD27⁺IgM⁻) (E) DN B cells (IgD⁻CD27⁻) (F) Plasmablasts (CD45+CD19+ and/or CD20+IgD−CD27+CD24−CD38hiCD138−). Biological replicates are shown as individual datapoints; horizontal bar represents the median value for each group. * and ** indicates statistical significance compared to healthy donor, where at least 3 biological replicates were available. *p<0.05 threshold for significance, **p<0.01. IIM samples in plasmablasts subset not tested for statistical significance due to low number of samples (n=2).

FIG. 5 shows the frequencies of CD19⁺ and/or CD20⁺ cells in B cells and select B cell subsets. CD19 and CD20 expression for selected B cell subsets shown from donors with LBCL (n=5), donors with LN (n=7), HD (n=14), donors with SSc (n=3) or SLE (n=7): Total B cells (CD45⁺Lineage⁻CD19⁺ and/or CD20⁺), DN age associated B cells (IgD⁻CD27⁻CD11c⁺), DN B cells (IgD⁻CD27⁻), Memory Switched B cells (IgD⁻CD27⁺IgM⁻) and Plasma blasts (IgD-CD27⁺CD24⁻CD38^{hi}CD138⁻). Individual datapoint required a minimum of 15 events (by flow cytometry) within any given subgroup to be reported.

FIG. 6A-C show cytokine production by T-cell products after 1 day of co-culture with antigen-positive and antigen-negative target cells. Levels of (A) IFN-γ, (B) IL-2, and (C) TNF-α secreted by T-cell products generated from Donors 1, 2, and 3 after 1 day of co-culture (1:1 effector: target) with dual antigen-positive (Nalm6 WT and Raji WT), single antigen-positive (Raji CD19KO, Raji CD20KO), or antigen-negative (Raji DKO) target cells, or in the absence of target cells (T cells alone). Quantitation of cytokine levels was performed using the Mesoscale Discovery® platform. These values lie within the limits of quantitation for this assay. Two multiplicities of infection (MOI; MOI=5 and MOI 10) were used to understand the relationship between the number of infectious viral particles per cell and transduction efficiency. Abbreviations: NTD, nontransduced; MOI, multiplicity of infection; IFN, interferon; IL, interleukin; TNF, tumor necrosis factor; WT, wild type; KO, knockout; DKO, double-antigen knockout.

FIG. 7A-F show cytokine production by KITE-001 products from patients with SLE at day 1 of co-culture with antigen-positive and antigen-negative target cells. Abbreviations: E:T, effector to target; IFN-γ, interferon-gamma; IL, interleukin; KO, knockout; NTD, nontransduced; TNF-α, tumor necrosis factor alpha; SLE, systemic lupus erythematosus. Results show levels of (A, D) IFN-γ, (B, E) IL-2, and (C, F) TNF-α pro-inflammatory cytokines secreted by KITE-001 T-cell products from SLE-248 and SLE-254 at Day 1 of co-culture with Raji cells (E:T 1:1), Raji CD19KO cells (E:T 1:1), Raji CD20KO cells (E:T 1:1), autologous B cells (E:T 2:1 and 1.5:1 for SLE-248 and SLE-254 respectively), K-562 (E:T 1:1) cells, or in the absence of target cells (T cells alone). Graphs denote mean±standard deviation. Cytokine measurements were performed using 3 technical replicates.

FIG. 8A-E show cytotoxicity of T-cell products against antigen-positive and antigen-negative target cell lines after 4 days co-culture. Two multiplicities of infection (MOI; MOI=5 and MOI 10) were used to understand the relationship between the number of infectious viral particles per cell and transduction efficiency.

FIG. 9A-H show the results of a luciferase-based killing assay. cytolytic activity of KITE-001 products from patients with SLE against antigen-positive and antigen-negative target cells at day 4 of co-culture. Figures show cytotoxicity of KITE-001 T-cell products on (A, E) Raji, (B, F) Raji CD19KO, (C, G) Raji CD20KO, or (D, H) K-562 target cell lines at 4 days post-co-culture at E:T ratios of 3:1, 1:1, 1:3, 1:9, 1:27, and 1:81 by luciferase assay. Graphs denote mean±standard deviation. Co-culture assays were performed using 3 technical replicates.

FIG. 10A-D show the results of a flow cytometry-based killing assay. cytolytic activity of KITE-001 products from patients with SLE against antigen-positive Raji cells and autologous B cells at day 4 of co-culture. Flow cytometry-based cytotoxicity of KITE-001 products from patients with SLE (SLE-248 and SLE-254) on (A, C) Raji (CD19⁺, CD20⁺) target cell line at 4 days post-coculture at E:T ratios of 3:1, 1:1, and 1:3. CAR T-cell products were co-cultured with (B, D) autologous B cells at E:T ratios of 2:1, 1:1.5, and 1:4.5 for SLE-248, and 1.5:1, 1:2, and 1:6 for SLE-254. Graphs denote mean±standard deviation. Co-culture assays were performed using 3 technical replicates.

FIG. 11 shows the effect of KITE-001 on the tumor burden of Raji-luc tumor-bearing NSG mice. Abbreviations: BLI, bioluminescence; IV, intravenous(ly); luc, luciferase; NSG, nonobese diabetic (NOD) severe combined immunodeficiency (scid) interleukin-2 receptor-gamma chain null. On Day 0.5×10⁵, Raji-luc cells were implanted IV. On Day 6, KITE-001 (1×10⁶ or 5×10⁶ CAR T cells) or NTD T cells were delivered by IV injection (6 mice per group). Mean BLI values are only represented when ≥80% of mice remained in each cohort (graph on the left). Tumor burden of individual animals is shown in graphs on the right. A significant (p<0.05; analysis of variance using Tukey's post-test) dose-dependent reduction in tumor burden was shown on Day 12 after treatment with KITE-001 compared with the vehicle and NTD controls.

FIG. 12 shows the effect of KITE-001 on tumor growth delay in Raji-luc tumor-bearing NSG mice. Abbreviations: BLI, bioluminescence; luc, luciferase; NSG, nonobese diabetic (NOD), severe combined immunodeficiency (scid), interleukin-2 receptor-gamma chain null; NTD, nontransduced. Tumor burden was monitored over time, and the statistical survival to the tumor burden endpoint of 1×10⁷ photons/sec was determined, with the percent surviving represented at each day of study. Significant (p<0.05; log-rank [Mantel-Cox]test) dose-dependent tumor growth delay was observed at both dose levels (1×10⁶ or 5×10⁶ CAR T cells) of KITE-001 compared with groups treated with vehicle or NTD T cells.

FIG. 13 shows in vivo expansion of total CAR⁺, CD4⁺, and CD8⁺ anti-CD19/CD20 CAR T Cells. Animals were administered 1×10⁶ or 5×10⁶ CAR T cells. Mean CAR⁺ cell count per microliter of murine blood sampled during the time course of the study. Mean CD4⁺ and CD8⁺ cells expressed as a percentage of the total CD3⁺ cells.

FIG. 14 shows total b-cell levels over time in participants with B-cell lymphomas treated with 0.5×10⁶, 1.0×10⁶, or 2.0×10⁶ CAR T cells/kg. Abbreviations: BLQ, Below Limit of Quantification; DL3, Dose Level 3; FL, follicular lymphoma; LBCL, large B-cell lymphoma; LEUKA, leukapheresis (pre-treatment baseline); NLPHL, nodular lymphocytic predominant Hodgkin lymphoma; tFL, transformed follicular lymphoma. Blood B cell levels over time from 9 participants presenting B-cell lymphomas (FL, LBCL, tFL, NLPHL) who had not received prior CAR T-cell therapy and presented detectable B cells before infusion with KITE-001 product. Within the first 2 to 4 weeks post KITE-001 treatment, all participants, at all 3 tested doses, presented complete B-cell depletion. In DL3 only 2 participants had assessment of B cell levels after Week 4.

FIG. 15 shows autologous B cell cytotoxicity by in vitro KITE-001 produced from autoimmune disease patients.

FIG. 16 shows antigen-specific cytotoxicity of KITE-001 against target cell lines in vitro.

FIG. 17 shows the KITE-001 cells' antigen-specific proliferation in vitro.

FIG. 18 shows antigen-specific cytokine production by KITE-001 in vitro.

FIG. 19 shows the viability of T-cell products generated from patients with myasthenia gravis (MG). NTD: nontransduced; MG: myasthenia gravis.

FIG. 20 shows the diameters of T-cell products generated from the MG patients.

FIG. 21 shows the expansion of T-cell products generated from the MG patients.

FIG. 22 shows the transduction efficiency of T-cell products generated from the MG patients.

FIG. 23 shows the proportions of CD4⁺ and CD8⁺ subsets between Days 0 and 9.

FIG. 24 shows the proportions of T-cell effector and memory subsets between Days 0 and 9.

FIG. 25 shows B cells detected from the MG patients and their CD19/CD20 expression.

FIG. 26 shows total CAR expression of T-cell products.

FIG. 27 shows cytolytic activity of T-cell products against antigen-positive and antigen-negative target cells at day 1 of co-culture.

FIG. 28 shows cytolytic activity of T-cell products against antigen-positive Raji cells and autologous B cells at day 1 of co-culture. Na*: Data not available due to lack of B cells.

FIG. 29 shows cytolytic activity of T-cell products against antigen-positive and antigen-negative target cells at day 4 of co-culture.

FIG. 30 shows cytolytic activity of T-cell products against antigen-positive Raji cells and autologous B cells at day 4 of co-culture. Na*: Data not available due to lack of B cells.

FIG. 31 shows cytokine production of T-cell products at day 1 of co-culture.

FIG. 32 shows proliferation of T-cell products. Na*: Data not available due to absence of B cells.

FIG. 33 shows the viability of T-cell products generated from patients with multiple sclerosis (MS). NTD: nontransduced; MS: multiple sclerosis.

FIG. 34 shows the diameters of T-cell products generated from the MS patients.

FIG. 35 shows the expansion of T-cell products generated from the MS patients.

FIG. 36 shows the transduction efficiency of T-cell products generated from the MS patients.

FIG. 37 shows the proportions of CD4⁺ and CD8⁺ subsets between Days 0 and 9.

FIG. 38 shows the proportions of T-cell effector and memory subsets between Days 0 and 9.

FIG. 39 shows B cells detected from the MG patients and their CD19/CD20 expression.

FIG. 40 shows total CAR expression of T-cell products.

FIG. 41 shows cytolytic activity of T-cell products against antigen-positive and antigen-negative target cells at day 1 of co-culture.

FIG. 42 shows cytolytic activity of T-cell products against antigen-positive Raji cells and autologous B cells at day 1 of co-culture. Na*: Data not available due to lack of B cells.

FIG. 43 shows cytolytic activity of T-cell products against antigen-positive and antigen-negative target cells at day 4 of co-culture.

FIG. 44 shows cytolytic activity of T-cell products against antigen-positive Raji cells and autologous B cells at day 4 of co-culture. Na*: Data not available due to lack of B cells.

FIG. 45 shows cytokine production of T-cell products at day 1 of co-culture.

FIG. 46 shows proliferation of T-cell products.

FIG. 47 shows the viability of T-cell products generated from patients with rheumatoid arthritis (RA). NTD: nontransduced; RA: rheumatoid arthritis.

FIG. 48 shows the diameters of T-cell products generated from the RA patients.

FIG. 49 shows the expansion of T-cell products generated from the RA patients.

FIG. 50 shows the transduction efficiency of T-cell products generated from the RA patients.

FIG. 51 shows the proportions of CD4⁺ and CD8⁺ subsets between Days 0 and 9.

FIG. 52 shows the proportions of T-cell effector and memory subsets between Days 0 and 9.

FIG. 53 shows total CAR expression of T-cell products.

FIG. 54 shows cytolytic activity of T-cell products against antigen-positive and antigen-negative target cells at day 1 of co-culture.

FIG. 55 shows cytolytic activity of T-cell products against antigen-positive Raji cells and autologous B cells at day 1 of co-culture.

FIG. 56 shows cytolytic activity of T-cell products against antigen-positive and antigen-negative target cells at day 4 of co-culture.

FIG. 57 shows cytolytic activity of T-cell products against antigen-positive Raji cells and autologous B cells at day 4 of co-culture.

FIG. 58 shows cytokine production of T-cell products at day 1 of co-culture.

FIG. 59 shows proliferation of T-cell products.

FIG. 60 shows the viability of T-cell products generated from patients with Sjogren's syndrome (SJ). NTD: nontransduced; SJ: Sjogren's syndrome.

FIG. 61 shows the diameters of T-cell products generated from the SJ patients.

FIG. 62 shows the expansion of T-cell products generated from the SJ patients.

FIG. 63 shows the transduction efficiency of T-cell products generated from the SJ patients.

FIG. 64 shows the proportions of CD4⁺ and CD8⁺ subsets between Days 0 and 9.

FIG. 65 shows the proportions of T-cell effector and memory subsets between Days 0 and 9.

FIG. 66 shows total CAR expression of T-cell products.

FIG. 67 shows cytolytic activity of T-cell products against antigen-positive and antigen-negative target cells at day 1 of co-culture.

FIG. 68 shows cytolytic activity of T-cell products against antigen-positive Raji cells and autologous B cells at day 1 of co-culture.

FIG. 69 shows cytolytic activity of T-cell products against antigen-positive and antigen-negative target cells at day 4 of co-culture.

FIG. 70 shows cytolytic activity of T-cell products against antigen-positive Raji cells and autologous B cells at day 4 of co-culture.

FIG. 71 shows cytokine production of T-cell products at day 1 of co-culture.

FIG. 72 shows proliferation of T-cell products.

FIG. 73 illustrates the structures of the anti-CD19 CAR and anti-CD20 CAR in a KITE-001 cell.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences.

The term “an equivalent nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference polypeptide or polynucleotide. In some aspects, the equivalent sequence retains the activity (e.g., epitope-binding) or structure (e.g., salt-bridge) of the reference sequence.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

A “single-chain variable fragment” or “scFv” refers to a fusion protein of the variable regions of the heavy (V_{H}) and light chains (V_{L}) of immunoglobulins. In some aspects, the regions are connected with a short linker peptide of ten to about 25 amino acids. The linker can be rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker. ScFv molecules are known in the art and are described, e.g., in U.S. Pat. No. 5,892,019.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgG₅, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgAQ1 and IgA2) or subclass of immunoglobulin molecule.

Light chains are classified as either kappa or lambda (K, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VK) and heavy (VH) chain portions determine antigen recognition and specificity. Conversely, the constant domains of the light chain (CK) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 and CK domains actually comprise the carboxy-terminus of the heavy and light chain, respectively.

As indicated above, the variable region allows the antibody to selectively recognize and specifically bind epitopes on antigens. That is, the VK domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. More specifically, the antigen-binding site is defined by three CDRs on each of the VH and VK chains (i.e. CDR-H1, CDR-H2, CDR-H3, CDR-L1, CDR-L2 and CDR-L3). In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen-binding domain are short, non-contiguous sequences of amino acids that are specifically positioned to form the antigen-binding domain as the antibody assumes its three dimensional configuration in an aqueous environment. The remainder of the amino acids in the antigen-binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see “Sequences of Proteins of Immunological Interest,” Kabat, E., et al., U.S. Department of Health and Human Services, (1983); and Chothia and Lesk, J. Mol. Biol., 196:901-917 (1987)).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

| | Kabat | Chothia |
|---|---|---|
| CDR-H1 | 31-35 | 26-32 |
| CDR-H2 | 50-65 | 52-58 |
| CDR-H3 | 95-102 | 95-102 |
| CDR-L1 | 24-34 | 26-32 |
| CDR-L2 | 50-56 | 50-52 |
| CDR-L3 | 89-97 | 91-96 |

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table above, the Kabat number system describes the CDR regions as follows: CDR-H1 begins at approximately amino acid 31 (i.e., approximately 9 residues after the first cysteine residue), includes approximately 5-7 amino acids, and ends at the next tryptophan residue. CDR-H2 begins at the fifteenth residue after the end of CDR-H1, includes approximately 16-19 amino acids, and ends at the next arginine or lysine residue. CDR-H3 begins at approximately the thirty third amino acid residue after the end of CDR-H2; includes 3-25 amino acids; and ends at the sequence W-G-X-G, where X is any amino acid. CDR-L1 begins at approximately residue 24 (i.e., following a cysteine residue); includes approximately 10-17 residues; and ends at the next tryptophan residue. CDR-L2 begins at approximately the sixteenth residue after the end of CDR-L1 and includes approximately 7 residues. CDR-L3 begins at approximately the thirty third residue after the end of CDR-L2 (i.e., following a cysteine residue); includes approximately 7-11 residues and ends at the sequence F or W-G-X-G, where X is any amino acid.

Antibodies disclosed herein may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks).

As used herein, the term “heavy chain constant region” includes amino acid sequences derived from an immunoglobulin heavy chain. A polypeptide comprising a heavy chain constant region comprises at least one of: a CH1 domain, a hinge (e.g., upper, middle, and/or lower hinge region) domain, a CH2 domain, a CH3 domain, or a variant or fragment thereof. For example, an antigen-binding polypeptide for use in the disclosure may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the disclosure comprises a polypeptide chain comprising a CH3 domain. Further, an antibody for use in the disclosure may lack at least a portion of a CH2 domain (e.g., all or part of a CH2 domain). As set forth above, it will be understood by one of ordinary skill in the art that the heavy chain constant region may be modified such that they vary in amino acid sequence from the naturally occurring immunoglobulin molecule.

The heavy chain constant region of an antibody disclosed herein may be derived from different immunoglobulin molecules. For example, a heavy chain constant region of a polypeptide may comprise a CH1 domain derived from an IgG₁ molecule and a hinge region derived from an IgG3 molecule. In another example, a heavy chain constant region can comprise a hinge region derived, in part, from an IgG₁ molecule and, in part, from an IgG3 molecule. In another example, a heavy chain portion can comprise a chimeric hinge derived, in part, from an IgG₁ molecule and, in part, from an IgG₄ molecule.

As used herein, the term “light chain constant region” includes amino acid sequences derived from antibody light chain. Preferably, the light chain constant region comprises at least one of a constant kappa domain or constant lambda domain.

A “light chain-heavy chain pair” refers to the collection of a light chain and heavy chain that can form a dimer through a disulfide bond between the CL domain of the light chain and the CH1 domain of the heavy chain.

As previously indicated, the subunit structures and three-dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat et al., U.S. Dept. of Health and Human Services, “Sequences of Proteins of Immunological Interest” (1983). The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen-binding regions to move independently. Hinge regions can be subdivided into three distinct domains: upper, middle, and lower hinge domains (Roux et al., J. Immunol 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CK regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (which may be intact, partial or modified in accordance with the instant disclosure) is obtained from a second species. In certain embodiments the target binding region or site will be from a non-human source (e.g., mouse or primate) and the constant region is human.

As used herein, “percent humanization” is calculated by determining the number of framework amino acid differences (i.e., non-CDR difference) between the humanized domain and the germline domain, subtracting that number from the total number of amino acids, and then dividing that by the total number of amino acids and multiplying by 100.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### Chimeric Antigen Receptors (CAR) Specific to CD20

Chimeric antigen receptors (CARs) are genetically engineered receptors. These engineered receptors can be readily inserted into and expressed by immune cells, such as T cells. A CAR can recognize a specific antigen and, when bound to that antigen, activates the immune cell to attack and destroy the cell bearing that antigen.

CARs can be engineered to bind to an antigen (such as a cell-surface antigen) by incorporating an antigen binding molecule that interacts with that targeted antigen. Preferably, the antigen binding molecule is an antibody fragment thereof, and more preferably one or more single chain antibody fragment (“scFv”). An scFv is a single chain antibody fragment having the variable regions of the heavy and light chains of an antibody linked together. It will be appreciated that the antigen binding molecule is typically contained within the extracellular portion of the CAR such that it is capable of recognizing and binding to the antigen of interest. Bispecific and multispecific CARs are contemplated within the scope of the invention, with specificity to more than one target of interest.

An example CAR includes, in addition to the antigen binding molecule, a transmembrane domain and an activation domain. In some embodiments, the CAR can further include a hinge domain between the antigen binding molecule and the transmembrane domain. In some embodiments, the CAR can further include a co-stimulatory domain.

In one embodiment, the antigen binding molecule in a CAR of the present disclosure is the anti-CD20 antibody Ab4 or the antigen-binding fragments thereof. Ab4 was identified in a batch of 36 fully-human antibodies identified from a full-length human IgG antibody library expressed in an in vitro yeast selection library. While four out of the 36 antibodies, Ab3, Ab4, Ab8 and Ab9, stood out as the best antibodies, it was discovered herein that Ab4 possessed the best properties for use in a CAR. As shown in Example 1, Ab4 exhibited the highest selectivity to CD20. Even though Ab4 also had some detectable binding to MUSK (muscle associated receptor tyrosine kinase) and THOP1 (thimet oligopeptidase 1), such binding did not lead to undesirable cytotoxicity (Example 2). In addition, membrane and cytoplasmic staining of mononuclear cells showed that any non-specific cross-reactivity of Ab4 in human tissues had little to no toxicologic significance (Example 3). These properties, therefore, underscore the suitability of Ab4 in constructing a CAR for cellular therapy.

In accordance with one embodiment of the present disclosure, therefore, provided is an anti-CD20 chimeric antigen receptor (CAR) that specifically binds CD20 which includes, among others, the VH and VL CDRs of Ab4. As shown in Table 3, the VH CDR1, VH CDR2, VH CDR3, VL CDR1, VL CDR2 and VL CDR3 of Ab4 include the amino acid sequences of SEQ ID NO:27-32, respectively.

In some embodiments, the anti-CD20 CAR includes a VH having at least 70%, 75%, 80%, 85%, 90%, 95%, 98%, 99% or 100% sequence identity to SEQ ID NO:12. In some embodiments, the anti-CD20 CAR includes a VL having at least 70%, 75%, 80%, 85%, 90%, 95%, 98%, 99% or 100% sequence identity to SEQ ID NO:13. In some embodiments, the anti-CD20 CAR includes a VH having the amino acid sequence of SEQ ID NO:12 and a VL having the amino acid sequence of SEQ ID NO:13.

In some embodiments, the anti-CD20 CAR includes a single chain fragment (scFv) derived from the Ab4 antibody, i.e., the VH and VL of Ab4 connected through a peptide linker. In some embodiments, the scFv has a configuration of VH-linker-VL. In some embodiments, the scFv has a configuration of VL-linker-VH. In some embodiments, the linker is a Whitlow linker (e.g., SEQ ID NO:6). In some embodiments, the scFv has at least 70%, 75%, 80%, 85%, 90%, 95%, 98%, 99% or 100% sequence identity to SEQ ID NO:14. In some embodiments, the scFv has at least 70%, 75%, 80%, 85%, 90%, 95%, 98%, 99% or 100% sequence identity to SEQ ID NO:14, while retaining the Ab4 CDRs (e.g., SEQ ID NO:27-32).

In some embodiments, the anti-CD20 CAR further includes a hinge, a transmembrane domain, an intracellular co-stimulatory domain, and/or an activating domain, each of which is discussed in further detail below.

### Activating Domains and Costimulatory Domains

The activation domain is part of the intracellular (cytoplasmic) portion of a CAR. The intracellular domain of a CAR can provide activation of at least one of the normal effector functions of the immune cell. Effector function of a T cell, for example, may refer to cytolytic activity or helper activity, including the secretion of cytokines.

A commonly used activation domain is the CD3zeta domain by itself or combined with any other desired intracellular domain(s). CD3 is an element of the T cell receptor on native T cells, and has been shown to be an important intracellular activating element in CARs. Example sequences of CD3zeta include SEQ ID NO:11 and sequences having at least 70%, 75%, 80%, 85%, 90%, 95%, 98% or 99% sequence identity to SEQ ID NO:11.

In some embodiments, the intracellular portion of the CAR further includes a costimulatory domain. An example is the signaling domain of 4-1BB, with an example sequence in SEQ ID NO:17 (or a sequence having at least 70%, 75%, 80%, 85%, 90%, 95%, 98% or 99% sequence identity to SEQ ID NO:17). In some embodiments, the costimulatory domain is N-terminal to the CD3zeta.

Another example is the signaling domain of CD28, with an example sequence in SEQ ID NO:10 (or a sequence having at least 70%, 75%, 80%, 85%, 90%, 95%, 98% or 99% sequence identity to SEQ ID NO:10). In some embodiments, the costimulatory domain is N-terminal to the CD3zeta.

### Transmembrane Domain and Hinge

Like other transmembrane proteins, a CAR also includes a transmembrane domain, which can be derived from, or includes, the transmembrane domain of another protein. Non-limiting examples of such proteins include CD28, CD8alpha, CD8beta, 4-1BB, B7-H3, BAFFR, BLAME, BTLA, CD100, CD103, CD11a, CD11b, CD11c, CD11d, CD160, CD18, CD19, CD19a, CD2, CD247, CD27, CD276, CD29, CD3 delta, CD3 epsilon, CD3 gamma, CD30, CD4, CD40, CD49a, CD49D, CD49f, CD69, CD7, CD79a, CD84, CD96, CDS, CEACAM1, CRT AM, DAP-10, DNAM1, Fc gamma receptor, GADS, GITR, HVEM, IA4, ICAM-1, ICOS, IL-2R beta, IL-2R gamma, IL-7R alpha, ITGA4, ITGA6, ITGAD, ITGAE, ITGAL, ITGAM, ITGAX, ITGB1, ITGB2, ITGB7, KIRDS2, LAT, LFA-1, LIGHT, LTBR, Ly9, NKG2C, NKG2D, NKp30, NKp44, NKp46, NKp80, OX-40, PAG, PD-1, PSGL1, SELPLG, SLAM, SLAMF4, SLAMF6, SLAMF7, SLP-76, TNFR2, TNFSFi4, TRANCE, VLA1, VLA-6, a cytokine receptor, a MHC class 1 molecule, a SLAM protein, a TNF receptor protein, a Toll ligand receptor, an activating NK cell receptor, an immunoglobulin protein, and an integrin.

In a particular example, the transmembrane domain is one from CD8α. Example sequences of CD8α transmembrane domain include SEQ ID NO:16 and sequences having at least 70%, 75%, 80%, 85%, 90%, 95%, 98% or 99% sequence identity to SEQ ID NO:16.

In another example, the transmembrane domain is one from CD28. Example sequences of CD28 transmembrane domain include SEQ ID NO:9 and sequences having at least 70%, 75%, 80%, 85%, 90%, 95%, 98% or 99% sequence identity to SEQ ID NO:9.

In some embodiments, the CAR further includes a hinge domain (or simply “hinge”) between the transmembrane domain and the antigen targeting molecule. The hinge domain can likewise be derived from cell surface proteins as listed above. In one example, the hinge domain is derived from CD8α. Example sequences of the CD8α hinge include SEQ ID NO:15 and sequences having at least 70%, 75%, 80%, 85%, 90%, 95%, 98% or 99% sequence identity to SEQ ID NO:15.

In another example, the hinge domain is derived from CD28. Example sequences of the CD28 hinge include SEQ ID NO:8 and sequences having at least 70%, 75%, 80%, 85%, 90%, 95%, 98% or 99% sequence identity to SEQ ID NO:8. In another example, the hinge domain is derived from IgG4.

In some embodiments, one or more spacers (e.g., AAA) can be inserted between any of the conjunctive domains as needed.

In some embodiments, the anti-CD20 CAR includes a scFv derived from Ab4, followed by a hinge domain from CD8 (e.g., SEQ ID NO:15 or a biological variant (i.e., a sequence having at least 70%, 75%, 80%, 85%, 90%, 95%, 98%, 99% or 100% sequence identity thereto)), a transmembrane domain from CD8 (e.g., SEQ ID NO:16 or a biological variant), a 4-1BB co-stimulatory domain (e.g., SEQ ID NO:17 or a biological variant) and a CD3 zeta activating domain (e.g., SEQ ID NO:11 or a biological variant).

In some embodiments, the anti-CD20 CAR has at least 70%, 75%, 80%, 85%, 90%, 95%, 98%, 99% or 100% sequence identity to SEQ ID NO:2. In some embodiments, the anti-CD20 CAR has at least 70%, 75%, 80%, 85%, 90%, 95%, 98%, 99% or 100% sequence identity to SEQ ID NO:2 while retaining the requisite domains or their biological equivalents as outlined above.

It is appreciated that an amino acid sequence can be modified to include those having one, two or three amino acid addition, deletion and/or substitutions while meeting the sequence identity requirement (e.g., 70%, 75%, 80%, 85%, 90%, 95%, 98%, 99% or 100% sequence identity). In some embodiments, the substitutions can be conservative substitutions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

It will also be understood by one of ordinary skill in the art that antibodies as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, 98%, or 99% identical to the starting sequence.

### Dual CAR-T Cells and Bicistronic CARs Specific to CD19/CD20

Bicistronic CARs and CAR-T cells having dual specificities are also provided. Such a dual CAR-T, referred to as “KITE-001”, was tested in the experimental examples. KITE-011 expressed an anti-CD19 CAR and an anti-CD20 CAR of the present disclosure. The anti-CD19 CAR includes a scFv derived from FMC63 (VH and VL as provided in SEQ ID NO:4 and 5, respectively), a CD28 hinge, a CD28 transmembrane domain, a CD28 intracellular, co-stimulatory domain, and a CD3 zeta activating domain.

As tested in the experimental examples, autologous KITE-001 products prepared from patients with SLE showed robust antigen-dependent and -specific proliferation from co-culture with antigen-positive target cells, including autologous B cells and demonstrated functionality with the production of pro-inflammatory cytokines such as IFN-7, IL-2, and TNF-α co-culture with dual antigen-positive target cells (Example 5). Also, importantly, KITE-001 exhibited excellent activity even when co-cultured with target cells expressing a single antigen, CD19 or CD20 (Example 5). Overall, KITE-001 showed robust antigen-dependent and -specific cytotoxicity against target B cells, suggesting its efficacy in treating cancers and autoimmune diseases mediated by dysregulated B cells.

In vivo study with KITE-001 demonstrated its potency in tumor growth inhibition and superior safety profile (Example 6) even at higher doses (e.g., 5×10⁶ CAR T cells in mice).

Interestingly, in huma cancer patients, KITE-001 exhibited increased expansion potential relative to axicabtagene ciloleucel which contains the anti-CD19 CAR only (Example 7). Moreover, KITE-001 products prepared from patients of various autoimmune diseases, e.g., systemic lupus erythematosus (SLE) with or without lupus nephritis (LN), systemic sclerosis (SSc), myasthenia gravis (MG), multiple sclerosis (MS), rheumatoid arthritis (RA) and Sjogren's Syndrome (SJ), were successfully prepared and exhibited excellent functional characteristics (Examples 9-13).

In accordance with one embodiment of the present disclosure, therefore, provided is an immune cell (e.g., T cell) that includes/expresses both an anti-CD19 CAR and an anti-CD20 CAR of the present disclosure. Various anti-CD20 CARs have been discussed above.

Similar to an anti-CD20 CAR, an anti-CD19 CAR of the present disclosure includes an antigen binding molecule, a hinge, a transmembrane domain, an intracellular co-stimulatory domain, and/or an activating domain, each of which is discussed in further detail below.

In some embodiments, an anti-CD19 chimeric antigen receptor (CAR) that specifically binds CD19 includes, among others, the VH and VL CDRs of FMC63. As shown in Table 3, the VH CDR1, VH CDR2, VH CDR3, VL CDR1, VL CDR2 and VL CDR3 of FMC63 include the amino acid sequences of SEQ ID NO:21-26, respectively.

In some embodiments, the anti-CD19 CAR includes a VH having at least 70%, 75%, 80%, 85%, 90%, 95%, 98%, 99% or 100% sequence identity to SEQ ID NO:4. In some embodiments, the anti-CD19 CAR includes a VL having at least 70%, 75%, 80%, 85%, 90%, 95%, 98%, 99% or 100% sequence identity to SEQ ID NO:5. In some embodiments, the anti-CD19 CAR includes a VH having the amino acid sequence of SEQ ID NO:4 and a VL having the amino acid sequence of SEQ ID NO:5.

In some embodiments, the anti-CD19 CAR includes a single chain fragment (scFv) derived from the FMC63 antibody, i.e., the VH and VL of FMC63 connected through a peptide linker. In some embodiments, the scFv has a configuration of VH-linker-VL. In some embodiments, the scFv has a configuration of VL-linker-VH. In some embodiments, the linker is a Whitlow linker (e.g., SEQ ID NO:6). In some embodiments, the scFv has at least 70%, 75%, 80%, 85%, 90%, 95%, 98%, 99% or 100% sequence identity to SEQ ID NO:7. In some embodiments, the scFv has at least 70%, 75%, 80%, 85%, 90%, 95%, 98%, 99% or 100% sequence identity to SEQ ID NO:7, while retaining the FMC63 CDRs (e.g., SEQ ID NO:21-26).

In some embodiments, the anti-CD19 CAR includes a scFv derived from FMC63, followed by a hinge domain from CD28 (e.g., SEQ ID NO:8 or a biological variant (i.e., a sequence having at least 70%, 75%, 80%, 85%, 90%, 95%, 98%, 99% or 100% sequence identity thereto)), a transmembrane domain from CD28 (e.g., SEQ ID NO:9 or a biological variant), a CD28 co-stimulatory domain (e.g., SEQ ID NO:10 or a biological variant) and a CD3 zeta activating domain (e.g., SEQ ID NO:11 or a biological variant).

In some embodiments, the anti-CD19 CAR has at least 70%, 75%, 80%, 85%, 90%, 95%, 98%, 99% or 100% sequence identity to SEQ ID NO:1. In some embodiments, the anti-CD19 CAR has at least 70%, 75%, 80%, 85%, 90%, 95%, 98%, 99% or 100% sequence identity to SEQ ID NO:1 while retaining the requisite domains or their biological equivalents as outlined above.

It is appreciated that an amino acid sequence can be modified to include those having one, two or three amino acid addition, deletion and/or substitutions while meeting the sequence identity requirement (e.g., 70%, 75%, 80%, 85%, 90%, 95%, 98%, 99% or 100% sequence identity). In some embodiments, the substitutions can be conservative substitutions.

It is appreciated that the anti-CD19 CAR and the anti-CD20 CAR in the immune cell can be encoded by a common vector/polynucleotide or separate ones. In a preferred embodiments, they are encoded in a bicistronic construct that includes an dual-specific open reading frame (ORF) encoding a bicistronic CAR.

Optionally, within the bicistronic CAR, between the anti-CD19 CAR and the anti-CD20 CAR, there is a cleavable linker and/or a ribosomal skipping signal. In some embodiments, the cleavable linker is a furin linker. In some embodiments, the ribosomal skipping signal is a 2A peptide, such as T2A. An example of combined cleavable linker/ribosomal skipping signal is provided in SEQ ID NO:19, and sequences having 70%, 75%, 80%, 85%, 90%, 95%, 98%, 99% or 100% sequence identity to SEQ ID NO:19.

In some embodiments, the bicistronic CAR has an anti-CD19 CAR-linker-anti-CAR20 CAR configuration. In some embodiments, the bicistronic ORF has an anti-CD20 CAR-linker-anti-CAR19 CAR configuration. In some embodiments, each of the CAR further includes a signal peptide (e.g., SEQ ID NO:18 and 20).

In some embodiments, the bicistronic CAR has at least 70%, 75%, 80%, 85%, 90%, 95%, 98%, 99% or 100% sequence identity to SEQ ID NO:3. In some embodiments, the bicistronic CAR has at least 70%, 75%, 80%, 85%, 90%, 95%, 98%, 99% or 100% sequence identity to SEQ ID NO:3 while retaining the requisite CARs and linkers, or their biological equivalents as outlined above.

Example polynucleotides and constructs are also provided that can encode, or be used to produce, the disclosed bicistronic CAR. One such example polynucleotide sequence is provided in SEQ ID NO:39 (Table 3). In some embodiments, the polynucleotide includes a nucleic acid sequence that has at least 70%, 75%, 80%, 85%, 90%, 95%, 98%, 99% or 100% sequence identity to SEQ ID NO:39. In some embodiments, the nucleic acid sequence that has at least 70%, 75%, 80%, 85%, 90%, 95%, 98%, 99% or 100% sequence identity to SEQ ID NO:39 retains structural characteristics as described herein, e.g., encoding amino acid sequences having the recited fragments/sequences as discussed above (e.g., CDR sequences). In some embodiments, the polynucleotide includes the nucleic acid sequence of SEQ ID NO:39.

Various types of immune cells have been tested for expressing CARs. Non-limiting examples include T cells, NK cells, macrophages and monocytes. A T cell, in some embodiment, may be an alpha beta T cell. In some embodiments, the T cell is a gamma delta T cell.

In some embodiments, T cells are classified as Helper T-cells (e.g., CD4+ cells), Cytotoxic T-cells (also known as TC, cytotoxic T lymphocyte, CTL, T-killer cell, cytolytic T cell, CD8+ T-cells or killer T cell), Memory T-cells ((i) stem memory T_{SCM} cells, like naive cells, are CD45RO−, CCR7+, CD45RA+, CD62L+(L-selectin), CD27+, CD28+ and IL-7Rα+, but they also express large amounts of CD95, IL-2Rβ, CXCR3, and LFA-1, and show numerous functional attributes distinctive of memory cells); (ii) central memory T_{CM} cells express L-selectin and the CCR7, they secrete IL-2, but not IFNγ or IL-4, and (iii) effector memory T_{EM} cells, however, do not express L-selectin or CCR7 but produce effector cytokines like IFNγ and IL-4), Regulatory T-cells (Tregs, suppressor T cells, or CD4+CD25+ regulatory T cells), Natural Killer T-cells (NKT) and Gamma Delta T-cells.

In some embodiments, the immune cell is autologous, or allogeneic. Without limitation, an autologous immune cell is obtained from a patient and engineered to express a CAR. An allogeneic immune cell may be derived from donor cells, such as stem cells.

Methods of preparing the engineered immune cells are also provided. In some embodiments, the method entails transfecting or transducing an immune cell with a polynucleotide or vector encoding the CAR or CARs.

### Treatments and Uses

The present disclosure is further directed to therapies which involve administering the CARs or CAR-immune cells of the disclosure to a patient such as an animal, a mammal, and a human for treating one or more of the disorders or conditions described herein.

In one embodiment, the CARs or CAR-immune cells of the disclosure can be used to treat or inhibit cancer. In some embodiments, the cancer cells in the patient express or overexpress CD19 and/or CD20.

In some embodiments, the immune cell was isolated from the cancer patient him- or her-self. In some embodiments, the cell was provided by a donor or from a cell bank. When the cell is isolated from the cancer patient, undesired immune reactions can be minimized.

Non-limiting examples of cancers include bladder cancer, breast cancer, colorectal cancer, endometrial cancer, esophageal cancer, head and neck cancer, kidney cancer, leukemia, liver cancer, lung cancer, lymphoma, melanoma, pancreatic cancer, prostate cancer, and thyroid cancer. In some embodiments, the cancer is one or more of gastric, pancreatic, esophageal, ovarian, and lung cancers.

In some embodiments, the cancer is acute lymphoblastic leukemia (ALL) (including non T cell ALL), acute myeloid leukemia, B cell prolymphocytic leukemia, B cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia, chronic or acute leukemia, diffuse large B cell lymphoma (DLBCL), follicular lymphoma (FL), hairy cell leukemia, Hodgkin's Disease, malignant lymphoproliferative conditions, MALT lymphoma, mantle cell lymphoma, Marginal zone lymphoma, monoclonal gammapathy of undetermined significance (MGUS), multiple myeloma, myelodysplasia and myelodysplastic syndrome, non-Hodgkin's lymphoma (NHL), plasma cell proliferative disorder (including asymptomatic myeloma (smoldering multiple myeloma or indolent myeloma), plasmablastic lymphoma, plasmacytoid dendritic cell neoplasm, plasmacytomas (including plasma cell dyscrasia; solitary myeloma; solitary plasmacytoma; extramedullary plasmacytoma; and multiple plasmacytoma), POEMS syndrome (also known as Crow-Fukase syndrome; Takatsuki disease; and PEP syndrome), primary mediastinal large B cell lymphoma (PMBC), small cell- or a large cell-follicular lymphoma, splenic marginal zone lymphoma (SMZL), systemic amyloid light chain amyloidosis, T cell acute lymphoid leukemia (“TALL”), T cell lymphoma, transformed follicular lymphoma, or Waldenstrom macroglobulinemia, Mantle cell lymphoma (MCL), Transformed follicular lymphoma (TFL), Primary mediastinal B cell lymphoma (PMBCL), Multiple myeloma, Hairy cell lymphoma/leukemia, or a combination thereof.

### Autoimmune Diseases and Conditions

The disclosed antibodies and CARs can also be used for treating autoimmune diseases and conditions. In some embodiments, provided are methods for treating an autoimmune disease or condition in a patient in need thereof. The method, in one embodiment, entails administering to the patient an effective amount of a CAR or CAR-immune cell of the present disclosure.

Examples of neurological (CNS, PNS, NMJ) autoimmune diseases (AID) include, without limitation, multiple sclerosis (MS), neuromyelitis optica spectrum disorder (NMOSD), NMDAR encephalitis, CIPD (Chronic Inflammatory Demyelinating Polyneurophathy) and myasthenia gravis.

Examples of AID in rheumatology include, without limitation, lupus nephritis (LN), non-renal systemic lupus erythematosus (SLE), systemic sclerosis (SSc), idiopathic inflammatory myopathies (IIM), Anti-neutrophil cytoplasmic antibody-associated (ANCA) vasculitis, Sjogren's syndrome, rheumatoid arthritis (RA), and pediatric lupus.

In some embodiments, the autoimmune disease or condition to be treated by the disclosed CARs or CAR-immune cells includes one or more of alopecia areata, autoimmune hemolytic anemia, autoimmune hepatitis, dermatomyositis, diabetes (type 1), celiac disease, autoimmune juvenile idiopathic arthritis, glomerulonephritis, Graves' disease, Guillain-Barré syndrome, idiopathic thrombocytopenic purpura, myasthenia gravis, autoimmune myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjogren's syndrome, systemic lupus erythematosus, autoimmune thyroiditis, Hashimoto's thyroiditis, autoimmune uveitis, vitiligo, and granulomatosis with polyangiitis (Wegener's).

In some embodiments, autoimmune disease or condition to be treated is relapsed/refractory or moderate to severe, progressive ones. Non-limiting examples include relapsed/refractory or moderate to severe, progressive systemic lupus erythematosus (SLE) with or without lupus nephritis (LN), systemic sclerosis (SSc), or idiopathic inflammatory myopathy (IIM).

Systemic lupus erythematosus (SLE) is a chronic, multisystem autoimmune disease of unknown cause, predominantly affecting women of childbearing age. The disease manifests in a wide array of symptoms, including general constitutional complaints, skin rashes, serositis, inflammatory arthritis, kidney disease, systemic vasculitis, lymph node enlargement, splenomegaly, hemolytic anemia, cognitive impairment, and central nervous system involvement. SLE is a severe and potentially life-threatening condition that can impact any organ system. It poses a significant morbidity and mortality while profoundly affecting patients' daily functioning and quality of life. SLE is associated with increased morbidity and mortality, with an estimated 10% to 15% of patients facing premature death due to lupus-related complications.

Persistent, uncontrolled disease activity over time leads to poorer outcomes, including organ damage and coronary artery disease. Approximately 50% to 60% of SLE patients develop lupus nephritis (LN) within the first 3 years of diagnosis, which significantly increases the risk of renal failure and death. The toxicities associated with both approved and off-label treatments for SLE contribute to the long-term morbidity and mortality observed in these patients.

Systemic sclerosis (SSc), also known as scleroderma, is a rare autoimmune connective tissue disease characterized by immune-mediated fibrosis, vasculopathy, and significant systemic involvement, leading to substantial morbidity and mortality. Common features include skin thickening, Raynaud's phenomenon, and digital ulcers. Systemic sclerosis is further categorized into ‘limited’ and ‘diffuse cutaneous’ SSc based on the extent of the skin involvement, with each having distinctive clinical and serologic features. However, the systemic nature of SSc is underscored by frequent extra-cutaneous manifestations, such as interstitial lung disease (ILD), pulmonary arterial hypertension (PAH), renal crisis, and gastrointestinal involvement. Diffuse cutaneous systemic sclerosis (dcSSc) is associated with high mortality resulting from early internal-organ involvement.

The estimated incidence of SSc ranges from 1 to 2 per 100,000 person-years, with a prevalence of 7 to 50 per 100,000 people, depending on the population studied. In a systematic review and meta-analysis analyzing the global incidence and prevalence of SSc, the pooled incidence rate of SSc was 1.4 per 100,000 person-years and the prevalence rate was 17.6 cases per 100,000 people. Regional epidemiological studies estimated higher numbers, with prevalence up to 44.3 per 100,000 people in North America. SSc is more common in women than in men, with a peak onset between the ages of 30 and 50. The prognosis for SSc varies widely based on the extent of internal organ involvement. ILD, in particular, is a significant predictor of mortality, and patients with ILD experience worse outcomes compared to those without lung involvement.

Idiopathic inflammatory myopathy (IIM), commonly referred to as myositis, are a group of rare autoimmune diseases primarily characterized by immune-mediated muscle injury and significant systemic involvement, leading to substantial morbidity and mortality. Common features include muscle weakness, low muscle endurance, and myalgia. However, the systemic nature of the disease is underscored by frequent extramuscular manifestations such as skin rashes, arthritis, ILD, and heart involvement.

The Bohan and Peter 1975 criteria and the ACR/European league against rheumatism (EULAR) 2017 criteria are both used in the diagnosis and classification of IIM. The criteria recognize 5 main types of IIM: dermatomyositis (DM), immune-mediated necrotizing myopathy (IMNM), anti-synthetase syndrome, inclusion body myositis (IBM), and polymyositis (PM), each distinguished by characteristic clinical, serologic, and pathological features. Many patients previously classified as having PM per the Bohan and Peter 1975 criteria are now reclassified into these subgroups, resulting in a decline in the number of patients considered to have PM.

Data indicate that PM, DM, and IMNM are more common in women, while IBM is more prevalent in men. The incidence of IIM increases with age, peaking around 50 years of age. The estimated incidence of IIM ranges from 0.2 to 2 per 100,000 person-years, with a prevalence of 2 to 25 per 100,000 people. However, these estimates vary widely depending on factors such as age, gender, geographical location, ethnicity, and study methodology. Mortality rates also show considerable variation, with 10-year survival rates ranging from 20% to 90% in studies from Europe, North America, and Japan. ILD is a significant prognostic factor in IIM, with mortality rates much higher in those with ILD compared to those without. Additionally, there is a strong association between adult-onset IIM and malignancy, particularly in patients with DM.

Rheumatoid arthritis (RA) is a long-term autoimmune disorder that primarily affects joints. It typically results in warm, swollen, and painful joints. Pain and stiffness often worsen following rest. Most commonly, the wrist and hands are involved, with the same joints typically involved on both sides of the body. The disease may also affect other parts of the body. While the cause of rheumatoid arthritis is not clear, it is believed to involve a combination of genetic and environmental factors. The underlying mechanism involves the body's immune system attacking the joints. This results in inflammation and thickening of the joint capsule. The goals of treatment are to reduce pain, decrease inflammation, and improve a person's overall functioning. Pain medications, steroids, and NSAIDs are frequently used to help with symptoms. A group of medications called disease-modifying antirheumatic drugs (DMARDs), such as hydroxychloroquine and methotrexate, may be used to try to slow the progression of disease.

Osteoarthritis (OA) is a type of joint disease that results from breakdown of joint cartilage and underlying bone. The most common symptoms are joint pain and stiffness. Initially, symptoms may occur only following exercise, but over time may become constant. Other symptoms may include joint swelling, decreased range of motion, and when the back is affected weakness or numbness of the arms and legs. Causes include previous joint injury, abnormal joint or limb development, and inherited factors. Risk is greater in those who are overweight, have one leg of a different length, and have jobs that result in high levels of joint stress. Osteoarthritis is believed to be caused by mechanical stress on the joint and low grade inflammatory processes. Treatment includes exercise, efforts to decrease joint stress, support groups, and pain medications.

Multiple sclerosis (MS) is a demyelinating disease in which the insulating covers of nerve cells in the brain and spinal cord are damaged. This damage disrupts the ability of parts of the nervous system to communicate, resulting in a range of signs and symptoms, including physical, mental, and sometimes psychiatric problems. Specific symptoms can include double vision, blindness in one eye, muscle weakness, trouble with sensation, or trouble with coordination. While the cause is not clear, the underlying mechanism is thought to be either destruction by the immune system or failure of the myelin-producing cells. There is no known cure for multiple sclerosis. Treatments attempt to improve function after an attack and prevent new attacks. In some embodiments, the multiple sclerosis is relapsing multiple sclerosis. In some embodiments, the multiple sclerosis is progressive multiple sclerosis.

Asthma is a common long-term inflammatory disease of the airways of the lungs. It is characterized by variable and recurring symptoms, reversible airflow obstruction, and bronchospasm. Symptoms include episodes of wheezing, coughing, chest tightness, and shortness of breath. Asthma is thought to be caused by a combination of genetic and environmental factors. Environmental factors include exposure to air pollution and allergens. Asthma is classified according to the frequency of symptoms, forced expiratory volume in one second (FEV1), and peak expiratory flow rate. It may also be classified as atopic or non-atopic, where atopy refers to a predisposition toward developing a type 1 hypersensitivity reaction. There is no cure for asthma. Symptoms can be prevented by avoiding triggers, such as allergens and irritants, and by the use of inhaled corticosteroids. Long-acting beta agonists (LABA) or antileukotriene agents may be used in addition to inhaled corticosteroids if asthma symptoms remain uncontrolled. Treatment of rapidly worsening symptoms is usually with an inhaled short-acting beta-2 agonist such as salbutamol and corticosteroids taken by mouth. In very severe cases, intravenous corticosteroids, magnesium sulfate, and hospitalization may be required.

Chronic obstructive pulmonary disease (COPD) is a type of obstructive lung disease characterized by long-term poor airflow. COPD can include two main conditions, emphysema and chronic bronchitis. In emphysema, the walls between many of the air sacs are damaged. As a result, the air sacs lose their shape and become floppy. This damage also can destroy the walls of the air sacs, leading to fewer and larger air sacs instead of many tiny ones. If this happens, the amount of gas exchange in the lungs is reduced. In chronic bronchitis, the lining of the airways stays constantly irritated and inflamed, and this causes the lining to swell. Lots of thick mucus forms in the airways, making it hard to breathe. There is no known cure for COPD, but the symptoms are treatable and its progression can be delayed.

Pain is a distressing feeling often caused by intense or damaging stimuli, such as stubbing a toe, burning a finger, putting alcohol on a cut, or bumping the “funny bone”. Pain is a complex, subjective phenomenon, defining pain has been a challenge. Pain is also referred to as an unpleasant sensory and emotional experience associated with actual or potential tissue damage. Pain is sometimes regarded as a symptom of an underlying condition, such as inflammation.

Myasthenia Gravis (MG) is a prototypical B-cell-mediated autoimmune disease characterized by the production of autoantibodies targeting proteins in the neuromuscular junction, namely the acetylcholine receptor (AChR) or muscle-specific kinase (MuSK). Notably, the thymus, where B cells play a role in T cell selection, often shows abnormalities in MG patients, such as the presence of germinal centers that support B-cell proliferation. B-cell tolerance checkpoint defects have been identified in AChR⁺ and MuSK⁺ MG patients and that poly- and/or auto-reactive naive B cells are proposed to serve as precursors to mature pathogenic autoantibody producing B cells.

### Doses and Dosing Schedule for Autoimmune Diseases and Conditions

A specific dosage and treatment regimen for any particular patient will depend upon a variety of factors, including the particular CAR or CAR-immune cell used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

Given the superior potency and safety profiles of KITE-001 and its excellent in vivo expansion capability, the instant inventors contemplate that a suitable dose for a CAR-T cell of the present disclosure is 0.2×10⁶ to 2×10⁶ cells per kilogram of body weight of the patient. In some embodiments, the dose is 0.1×10⁶ cells, 0.2×10⁶ cells, 0.3×10⁶ cells, 0.4×10⁶ cells, 0.5×10⁶ cells, 0.6×10⁶ cells, 0.7×10⁶ cells, 0.8×10⁶ cells, 0.9×10⁶ cells, 1.0×10⁶ cells, 1.1×10⁶ cells, 1.2×10⁶ cells, 1.3×10⁶ cells, 1.4×10⁶ cells, 1.5×10⁶ cells, 1.6×10⁶ cells, 1.7×10⁶ cells, 1.8×10⁶ cells or higher per kilogram of body weight of the patient. In some embodiments, the dose is not greater than 5×10⁶ cells, 4×10⁶ cells, 3×10⁶ cells, 2×10⁶ cells, 1.9×10⁶ cells, 1.8×10⁶ cells, 1.7×10⁶ cells, 1.6×10⁶ cells, 1.5×10⁶ cells, 1.4×10⁶ cells, 1.3×10⁶ cells, 1.2×10⁶ cells, 1.1×10⁶ cells, 1.0×10⁶ cells, 0.9×10⁶ cells, 0.8×10⁶ cells, 0.7×10⁶ cells, 0.6×10⁶ cells, 0.5×10⁶ cells, 0.4×10⁶ cells, or 0.3×10⁶ cells.

In some embodiments, the dose is 0.1×10⁶ to 0.9×10⁶ cells per kilogram of body weight.

In some embodiments, the dose is 0.2×10⁶ to 0.8×10⁶ cells per kilogram of body weight. In some embodiments, the dose is 0.3×10⁶ to 0.7×10⁶ cells per kilogram of body weight. In some embodiments, the dose is 0.4×10⁶ to 0.6×10⁶ cells per kilogram of body weight. In some embodiments, the dose is 0.45×10⁶ to 0.55×10⁶ cells per kilogram of body weight. In some embodiments, the dose is about 0.5×10⁶ cells per kilogram of body weight.

In some embodiments, the dose is 0.5×10⁶ to 1.5×10⁶ cells per kilogram of body weight.

In some embodiments, the dose is 0.6×10⁶ to 1.4×10⁶ cells per kilogram of body weight. In some embodiments, the dose is 0.7×10⁶ to 1.3×10⁶ cells per kilogram of body weight. In some embodiments, the dose is 0.8×10⁶ to 1.2×10⁶ cells per kilogram of body weight. In some embodiments, the dose is 0.9×10⁶ to 1.1×10⁶ cells per kilogram of body weight. In some embodiments, the dose is 0.95×10⁶ to 1.05×10⁶ cells per kilogram of body weight. In some embodiments, the dose is about 1.0×10⁶ cells per kilogram of body weight.

In some embodiments, the dose does not exceed a total of 1×10⁸ cells, 1.5×10⁸ cells or 2×10⁸ cells per patient regardless of body weight.

In one embodiment, the dosing is just once per treatment. In one embodiment, the dosing is no more than once per month, every 4 months, every 6 months, every 12 months, every 2 years, every 3 years, every 4 years, every 5 years, or every 10 years.

Methods of administration of the CARs or CAR-immune cells include but are not limited to intradermal, intramuscular, intraperitoneal, intravenous, subcutaneous, intranasal, epidural, and oral routes. The antigen-binding polypeptides or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered orally, rectally, parenterally, intracistemally, intravaginally, intraperitoneally, topically (as by powders, ointments, drops or transdermal patch), bucally, or as an oral or nasal spray.

The term “parenteral” as used herein refers to modes of administration which include intravenous, intramuscular, intraperitoneal, intrasternal, subcutaneous and intra-articular injection and infusion.

Administration can be systemic or local. In addition, it may be desirable to introduce the antibodies of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It may be desirable to administer the CARs or CAR-immune cells or compositions of the disclosure locally to the area in need of treatment; this may be achieved by, for example, and not by way of limitation, local infusion during surgery, topical application, e.g., in conjunction, with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the disclosure, care must be taken to use materials to which the protein does not absorb.

The amount of the CARs or CAR-immune cells of the disclosure which will be effective in the treatment, inhibition and prevention of an immune or malignant disease, disorder or condition can be determined by standard clinical techniques. In addition, in vitro assays may optionally be employed to help identify optimal dosage ranges. The precise dose to be employed in the formulation will also depend on the route of administration, and the seriousness of the disease, disorder or condition, and should be decided according to the judgment of the practitioner and each patient's circumstances. Effective doses may be extrapolated from dose-response curves derived from in vitro or animal model test systems.

### EXAMPLES

### Example 1: Comparison of Anti-CD20 Binders

Thirty six fully human antibodies were isolated from a full-length human IgG antibody library expressed in an in vitro yeast selection library that was screened for CD20 binding. Four top candidates were selected for further testing. The binding of the four anti-CD20 antibodies to CD20 and 5647 other human proteins (the vast majority of which are cell surface membrane proteins) was determined in this example, to evaluate the antibodies' binding specificity to CD20. Anti-CD20 antibody Rituxan and Leu16 scFv-Fc (Leu16sscFv) were used as benchmarks and an IgG protein was used as negative control.

TABLE 1
Anti-CD20 Binders

| Name | Sequence | SEQ ID NO: |
|---|---|---|
| Ab3 VH | QLQLQESGPGLVKPSETLSLTCTVSGGSISSSSYYWGWIRQPPGKGLEWIG | 33 |
| | SIYYSGSTYYNPSLKSRVTISVDTSKNQFSLKLSSVTAADTAVYYCARETD | |
| | YSSGMGYGMDVWGQGTTVTVSS | |
| Ab3 VL | DIQMTQSPSSLSASVGDRVTITCRASQSINSYLNWYQQKPGKAPKLLIYAA | 34 |
| | SSLQSGVPSRFSGSGSGTDFTLTISSLQPEDFATYYCQQSLADPFTFGGGT | |
| | KVEIK | |
| Ab4 VH | QVQLVQSGAEVKKPGASVKVSCKASGYTFKEYGISWVRQAPGQGLEWMGWI | 12 |
| | SAYSGHTYYAQKLQGRVTMTTDTSTSTAYMELRSLRSDDTAVYYCARGPHY | |
| | DDWSGFIIWFDPWGQGTLVTVSS | |
| Ab4 VL | DIQMTQSPSSLSASVGDRVTITCRASQSISSYLNWYQQKPGKAPKLLIYAA | 13 |
| | SSLQSGVPSRFSGSGSGTDFTLTISSLQPEDFATYYCQQSYRFPPTFGQGT | |
| | KVEIK | |
| Ab8 VH | QVQLQQWGAGLLKPSETLSLTCAVYGGSFSRYVWSWIRQPPGKGLEWIGEI | 35 |
| | DSSGKTNYNPSLKSRVTISVDTSKNQFSLKLSSVTAADTAVYYCARVRYDS | |
| | SDSYYYSYDYGMDVWGQGTTVTVSS | |
| Ab8 VL | DIVLTQSPDSLAVSLGERATINCKSSQSVLYSSNNKNYLAWYQQKPGQPPK | 36 |
| | LLIYWASSRESGVPDRESGSGSGTDFTLTISSLQAEDVAVYYCQQSYSFPW | |
| | TFGGGTKVEIK | |
| Ab9 VH | QVQLQQWGAGLLKPSETLSLTCAVYGGSFSGYAWSWIRQPPGKGLEWIGEI | 37 |
| | DHRGFTNYNPSLKSRVTISVDTSKNQFSLKLSSVTAADTAVYYCARVRYDS | |
| | SDSYYYSYDYGMDVWGQGTTVTVSS | |
| Ab9 VL | DIVLTQSPDSLAVSLGERATINCKSSQSVLYSSNNKNYLAWYQQKPGQPPK | 38 |
| | LLIYWASSRESGVPDRFSGSGSGTDFTLTISSLQAEDVAVYYCQQSYSFPW | |
| | TFGGGTKVEIK | |

These clones are referred to as Ab3, Ab, Ab8 and Ab9 and their VH and VL sequences are provided in Table 1. The testing molecules include a scFv fused to a Fc fragment. Investigation of the levels of binding of each test molecule to fixed, untransfected human HEK293 cells, and to cells over-expressing CD2 or each of the 5647 proteins, determined the appropriate screening concentration for each test molecule. This revealed 43 primary hits altogether, including CD2.

Each primary hit was re-expressed, along with a control receptor (EGFR), and fixed cells were re-tested with each test molecule, and a series of positive and negative controls. After removing non-specific, non-reproducible, and likely intracellular proteins, there remained multiple specific cell surface hits for the test molecules. All four test molecules interacted with CD20, their primary target.

The levels of binding of each of these test molecules to CD20 and other proteins with at least weak binding are summarized in Table 2. Among all these binders, Ab4 had the strongest binding to CD20 and the least binding to non-target proteins.

TABLE 2
Binding Results

| Clone | Gene ID | | Primary | | Confirmation | |
|---|---|---|---|---|---|---|
| (conc) | (Accession) | Protein Name | 1 | 2 | 1 | 2 |
| Ab4 | MS4A1 | Membrane spanning 4- | ***** | ***** | ***** | ***** |
| (3 μg/ml) | (BC002807) | domains A1 (CD20) | | | | |
| | MUSK | Muscle associated receptor | **/*** | **/*** | ** | ** |
| | (NM_005592.3) | tyrosine kinase | | | | |
| Ab3 | MS4A1 | Membrane spanning 4- | ** | ** | ** | ** |
| (5 μg/ml) | (BC002807) | domains A1 (CD20) | | | | |
| | KIR2DS1 | Killer cell | **/*** | **/*** | ***** | ***** |
| | (NM_014512) | immunoglobulin like | | | | |
| | | receptor, two Ig domains | | | | |
| | | and short cytoplasmic | | | | |
| | | tail 1 | | | | |
| | KIK2DL2 | Killer cell | */** | */** | ** | ** |
| | (NM_014219) | immunoglobulin like | | | | |
| | | receptor, two Ig domains | | | | |
| | | and long cytoplasmic | | | | |
| | | tail 2 | | | | |
| | KIR2DL1 | Killer cell | ***** | ***** | ***** | ***** |
| | (NM_014218.2) | immunoglobulin like | | | | |
| | | receptor, two Ig domains | | | | |
| | | and long cytoplasmic | | | | |
| | | tail 1 | | | | |
| | KIR2DL1 | Killer cell | | | | |
| | (BC069344.1) | immunoglobulin like | | | | |
| | | receptor, two Ig domains | | | | |
| | | and long cytoplasmic | | | | |
| | | tail 1 | | | | |
| | SECTM1 | Secreted And | */** | */** | ***** | ***** |
| | (BC017716) | Transmembrane 1 | | | | |
| | FLRT3 | Fibronectin leucine rich | ** | ** | *** | *** |
| | (NM_013281.3) | transmembrane protein 3 | | | | |
| | IL6R | Interleukin 6 receptor | */** | */** | ** | |
| | (NM_00565.2) | | | | | |
| | ADGRB1 | Adhesion g protein- | ** | ** | ** | *** |
| | (NM_001702) | coupled receptor B1 | | | | |
| | NFASC | Neurofascin | **/*** | **/*** | *** | *** |
| | (NM_001005388.2) | | | | | |
| | IGSF3 | Immunoglobulin | **/*** | **/*** | *** | *** |
| | (NM_001542.2) | superfamily member 3 | | | | |
| | L1CAM | L2 cell adhesion | ***/***** | ***/***** | *** | ***** |
| | (NM_000425.3) | molecule | | | | |
| Ab8 | MS4A1 | Membrane spanning 4- | **/*** | **/*** | *** | *** |
| (19.5 μg/ml) | (BC002807) | domains A1 (CD20) | | | | |
| | HAVCR2 | Hepatitis A virus | **/*** | **/*** | *** | *** |
| | (BC063431.1) | cellular receptor 2 | | | | |
| | FCGR3B | Fc fragment of IgG | ** | ** | ** | ** |
| | (NM_000570.3) | receptor IIIb | | | | |
| | FCGR3A | Fc fragment of IgG | **/*** | **/*** | ** | ** |
| | (BC017865.1) | receptor IIIa | | | | |
| | KIR2DL1 | Killer cell | | | ** | ** |
| | (NM_014218.2) | immunoglobulin like | | | | |
| | | receptor, two Ig domains | | | | |
| | | and long cytoplasmic | | | | |
| | | tail 1 | | | | |
| | CD19 | CD19 molecule | ** | **/*** | ***** | ***** |
| | (BC006338) | | | | | |
| Ab9 | MS4A1 | Membrane spanning 4- | ***/***** | ***/***** | *** | *** |
| (18 μg/ml) | (BC002807) | domains A1 (CD20) | | | | |
| | FCGR2B | Fc fragment of IgG | *** | *** | ** | ** |
| | (BC031992.1) | receptor IIb | | | | |
| | FCGR3A | Fc fragment of IgG | **/*** | **/*** | ** | |
| | (BC017865.1) | receptor IIIa | | | | |
| | FCGR3B | Fc fragment of IgG | | | ** | ** |
| | (NM_000570.3) | receptor IIIb | | | | |
| Rituxan | MS4A1 | Membrane spanning 4- | ***** | ***** | ***** | ***** |
| (10 μg/ml) | (BC002807) | domains A1 (CD20) | | | | |
| | AHCY | Adenosylhomocysteinase | | | ** | ** |
| | (BC010018) | | | | | |
| | TULP3 | Tubby like protein 3 | ** | ** | ** | *** |
| | (BC032587.1) | | | | | |
| Leu16sscFv | MS4A1 | Membrane spanning 4- | ***/***** | ***/***** | *** | *** |
| (17 μg/ml) | (BC002807) | domains A1 (CD20) | | | | |
| | GPR173 | G protein-coupled | ** | ** | *** | *** |
| | (BC009861) | receptor 173 | | | | |
| | CD33 | CD33 molecule | ** | ** | *** | *** |
| | (BC028152) | | | | | |
| | CD33 | CD33 molecule | **/*** | **/*** | ***** | *** |
| | (NM_001082618.1) | | | | | |
| | GPR173 | G protein-coupled | ** | ** | ** | ** |
| | (NM_018969.4) | receptor 173 | | | | |
| | TNFRSF17 | TNF receptor superfamily | * | * | ** | ** |
| | (BC058291.1) | member 17 (BCMA) | | | | |
| | SIGLEC12 | Sialic acid binding Ig | ***/***** | ***/***** | ***** | ***** |
| | (NM_053003.2) | like lectin 12 | | | | |

Note:
*****: strong;
***: medium;
**: weak;
*: very week.

### Example 2. Binding Specificity of Ab4

Example 1 showed that Ab4 exhibited some weak binding to MUSK. In addition, there was detectable level of binding to THOP1 (thimet oligopeptidase 1). THOP1 is an intracellular cytosolic kinase and is not expressed extracellularly. Of interest, THOP1 was also identified as a medium strength binding hit in the IgG control and was therefore considered not likely to be a biologically relevant hit. MUSK is a cell-surface receptor tyrosine kinase required for the formation and maintenance of the neuromuscular junction.

This example tested the activity of a chimeric antigen receptor (CAR) containing a Ab4 scFv in inducing cytotoxicity against HEK293 T cells overexpressing CD20 or variants of the MUSK protein as well a THOP1 protein.

Several isoforms of MUSK arising from alternative splicing has been described. These isoforms differ by the presence of either 1 or 2 different insertions (a 10 amino acid insertion and a 9 amino acid insertion) in the ectodomain. Luciferase-expressing human embryonic kidney 293T (HEK293T) cells were transduced with a lentiviral vector (LVV) encoding MUSK variant 1, MUSK variant 2, MUSK variant 3, THOP1, or CD20 (as a positive control). All proteins were tagged with green fluorescent protein (GFP) for ease of detection. Transduced cells were enriched by cell sorting based on GFP expression and post enrichment cultures were confirmed to be greater than 80% GFP+ for all HEK293T overexpression cell lines generated. Overexpression of the gene of interest was confirmed by quantitative polymerase chain reaction, Western blotting, and flow cytometry. Engineered HEK293T cells and nonengineered parental HEK293 T cells (controls) were subsequently co-cultured with T cells transduced to express the Ab4-harboring anti-CD20 CAR. CAR T-cell-mediated cytotoxicity was measured on Days 1 and 4 after co-culture initiation.

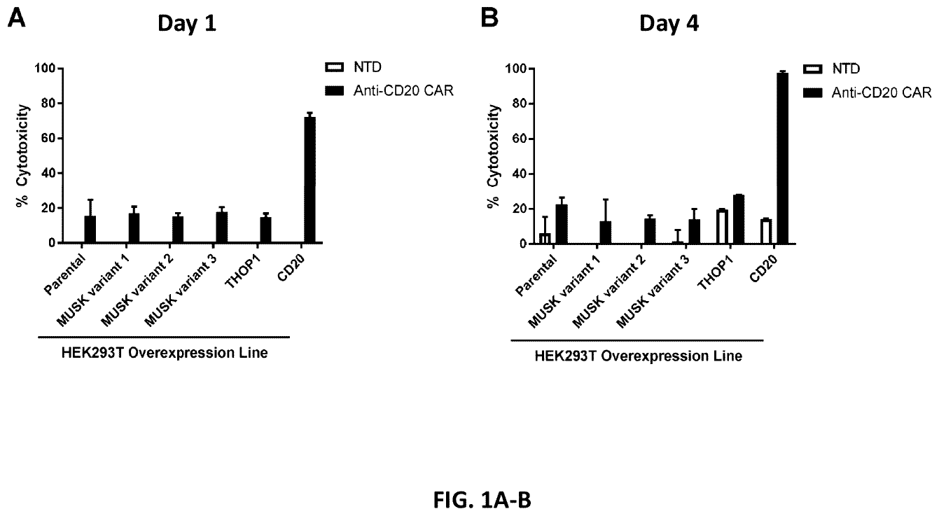

HEK293T cells that overexpressed GFP-tagged MUSK variant 1, MUSK variant 2, MUSK variant 3, THOP1, or CD20 were successfully generated. Anti-CD20 CAR T-cells with Ab4 demonstrated high levels of cytotoxicity to HEK293T cells overexpressing CD20 proteins (FIG. 1).

In contrast, anti-CD20 CAR T cells co-cultured with HEK293T cells overexpressing the MUSK variants or THOP1 resulted in levels of cytotoxicity that were similar to that resulting from co-culture with the non-transduced parental HEK293T control cell line (FIG. 1).

This in vitro assessment using an overexpression HEK293T system, therefore, invalidated the off-target protein hits identified in Example 1, and confirmed specificity of Ab4 to CD20.

### Example 3. Cross-Reactivity in Normal Human Tissues

In this example, the potential cross reactivity with nontarget normal human tissue of Ab4 was explored by immunohistochemistry using cryosections of normal human tissue.

Human tissues were collected from surgical or autopsy specimens and consisted of a panel of 36 different tissue types, each collected from at least 3 separate donors. Cryosections of cell pellets of Raji (high CD20-expressing [CD20^{high}]) and of Nalm6 (low CD20-expressing [CD20^{low}]) immortalized cell lines were used as positive controls, Raji CD20 knockout (KO; CD20⁻) cell pellets were used as a negative control, and human tonsil mononuclear cells served as an ancillary control due to the known population of CD20⁺ B cells in this tissue.

The Ab4-Fc displayed moderate to intense membrane and cytoplasmic staining in the positive control CD20^{high} Raji cells at the 2 concentrations tested, 1 and 5 μg/mL. At 5 μg/mL, Ab4-Fc produced weak to strong membrane staining of the CD20^{low} Nalm6 cells that reduced to weak-to-moderate and rare-to-occasional at the lower concentration (1 μg/mL). The Ab4-Fc did not react with the negative control Raji CD20 KO cells at either concentration. The specific reactions of Ab4-Fc with the positive control materials and the lack of specific reactivity with the negative control material and the control article, showed that the assay was sensitive, specific, and reproducible.

Membrane and cytoplasmic staining of mononuclear cells with Ab4-Fc in most human tissues, including lymphocytes in blood smears, was consistent with the known expression of CD20 by B cells. All other reactivity in the human tissue panel with Ab4 was limited to the cytoplasm and/or cytoplasmic granules, which, in tissue cross-reactivity studies, is considered to have little to no toxicologic significance due to the limited ability of antibody-based therapeutics to access the cytoplasmic compartment in vivo.

### Example 4. CD19 and CD20 Expression on B Cells from Autoimmune Patients

This example characterized B-cell subsets and CD19/CD20 expression in PBMCs by flow cytometry from patients with SLE (systemic lupus erythematosus) with LN (lupus nephritis) (LN; n=7) or without LN (nrSLE; n=7), SSc (systemic sclerosis; n=6), IIM (idiopathic inflammatory myopathy; n=3), HDs (healthy donors; n=14), and patients with diffuse large B cell lymphoma (LBCL; n=5).

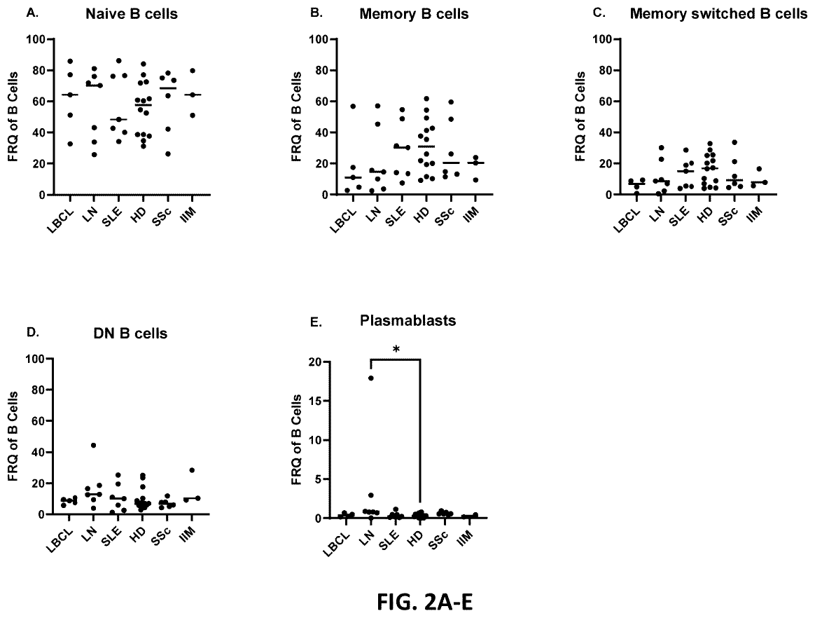

While there was a wide variability of B-cell subset frequencies (FRQ=% of B cells) across samples, overall the subset frequencies observed for the autoimmune indications were not significantly different from those of HDs (FIG. 2), with the notable exception of plasmablasts (CD27+/IgD−/CD38+/CD138−), which were significantly (p<0.05 by descriptive statistics) elevated in patients with LN compared to HDs (FIG. 2). Albeit the differences were not significant by descriptive statistics, median memory B-cell (CD27⁺) frequencies (% of B cells) appeared reduced in patients with LN, SSc, and IMM compared with HDs. Conversely, median naive B-cell (CD27−/IgD+/CD38low/CD24low) frequencies appeared elevated in patients with SSc. DN memory B cells (IgD⁻CD27⁻), which include age-associated B cells (CD11c+) and are associated with autoimmune diseases and chronic inflammation, appeared elevated in patients with LN compared with HDs.

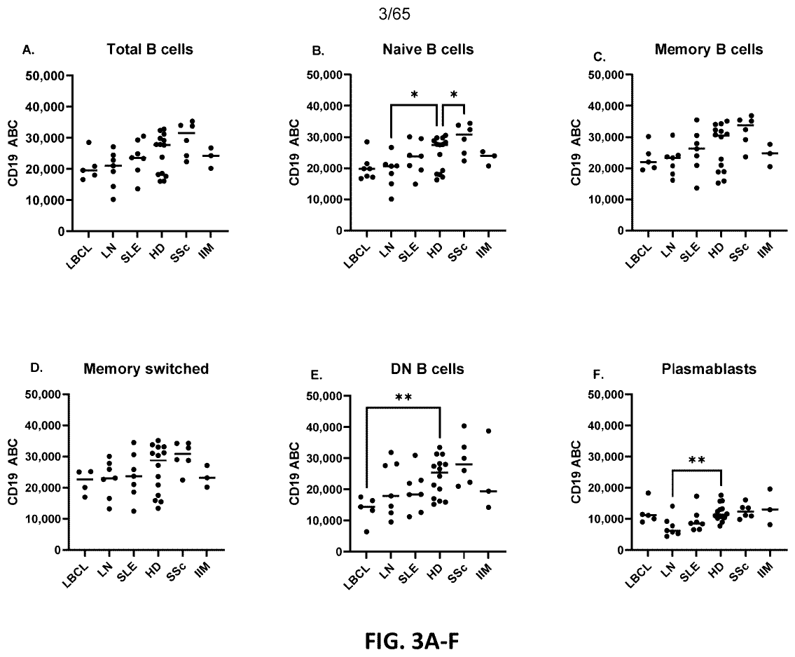

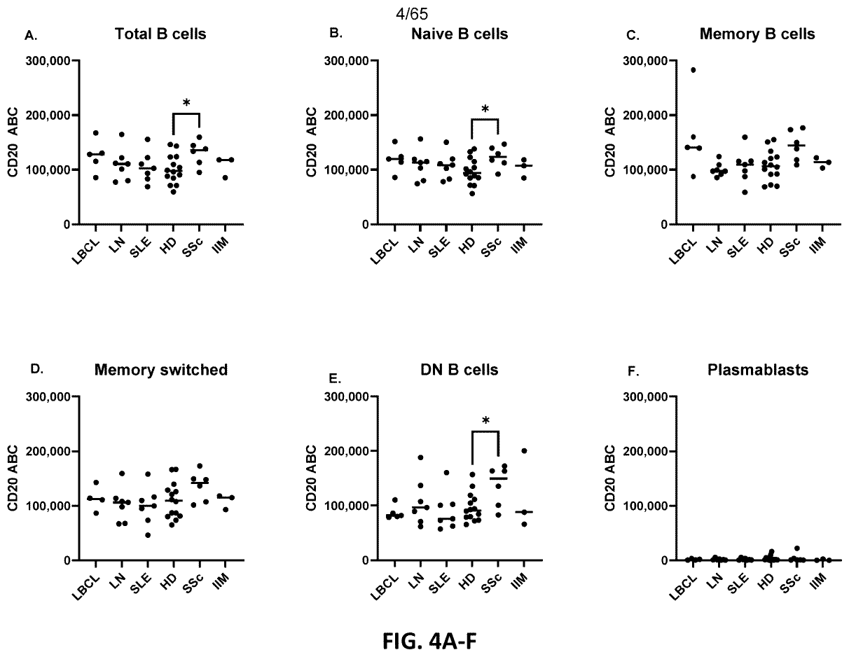

The expression of CD19 and CD20 was calculated as antibodies bound per cell; and in all B-cell subsets, including naive, memory, plasmablasts, and DN memory B cells, was highly variable, but overall within the ranges observed in samples from HDs and from patients with LBCL, with few exceptions (FIG. 3-4). Lower CD19 expression was observed in plasmablasts and naive B cells from patients with LN (p<0.05; FIG. 3), compared to HDs, but in similar range to that observed for patients with LBCL. Patients with SSc presented elevated CD19 and CD20 on naive B cells and elevated CD20 expression on DN memory B cells (FIGS. 2 and 4).

In all four autoimmune indications, the median B-cell expression of CD20 was between 4.3- and 5.3-fold higher than the median expression of CD19 (FIG. 3). Consistent results were observed in HDs and patients with LBCL and across all B-cell subsets, except for plasmablasts, where CD19 expression was between 6.7- and 16.4-fold higher than that of CD20.

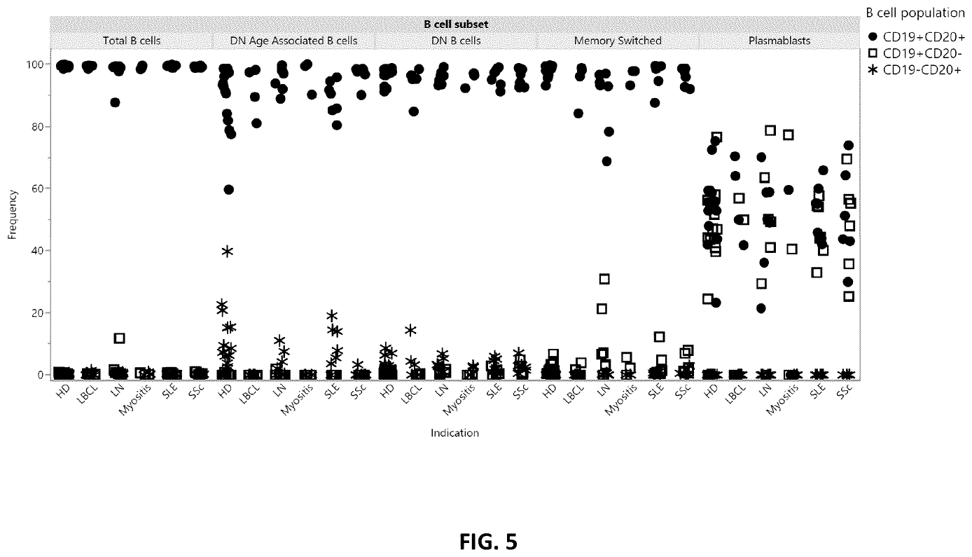

While overall most of the B cells exhibit co-expression of CD19 and CD20 (CD19⁺CD20⁺ B cells >85% for all donors; FIG. 5), subsets of both CD19⁺CD20⁻ and CD19⁻CD20⁺ B cells were observed in all 4 autoimmune conditions, similarly to HD and LBCL donors. Across the autoimmune conditions, plasmablasts were abundantly CD19⁺CD20⁻, with median frequencies of 40% to 50% (48% in patients with LN; 44% in patients with nrSLE; 50% in patients with SSc; and 40% in patients with IIM; FIG. 5). The memory switched (CD27⁺IgD⁻/IgM⁻) subset presented a median of 6.6%, 1.1%, 1.8%, and 2.2% of CD19⁺CD20⁻ B cells in patients with LN, nrSLE, SSc, and IIM, respectively, with 3/14 (21%) of nrSLE and LN donors presenting >10% frequencies. Albeit CD19⁻CD20⁺ frequencies were generally low (<40% for all donors across all B cell subsets), the DN subset, including age-associated B cells, presented a clear enrichment of CD19⁻CD20⁺ cells compared to other B cell subsets, particularly in nrSLE and LN donors, where 7/14 (50%) donors showed >5% frequencies (FIG. 5).

Overall, variable expression of CD19 and/or CD20 across B-cell subsets supports targeting both antigens for comprehensive B-cell depletion.

### Example 5. Preparation of KITE-001 and In Vitro Pharmacology Studies

A construct encoding an anti-CD19 CAR and an anti-CD20 CAR was synthesized and cloned into a replication-deficient LVV (lentiviral) backbone. The complete construct is referred to as an anti-CD19/CD20 CAR (or a bicistronic CAR) and is the dual antigen-targeting CAR component of “KITE-001”.

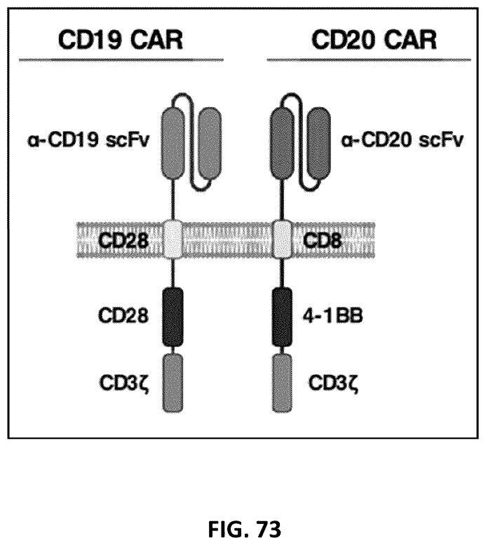

The anti-CD19 CAR included a scFv derived from the FMC63 antibody and the anti-CD20 CAR included a scFv derived from Ab4 as described above. The sequences of these CARs and their individual domains are shown in Table 3 below. Importantly, the anti-CD19 CAR included a CD28 intracellular signaling domain and the anti-CD20 CAR included a 4-1BB intracellular signaling domain (see illustration of the CAR domains in FIG. 73 and sequence in SEQ ID NO:3).

TABLE 3
Sequences and CARs and Components

| Name | Sequence | SEQ ID NO: |
|---|---|---|
| Anti-CD19 CAR | DIQMTQTTSSLSASLGDRVTISCRASQDISKYLNWYQQKPDGTVKLLIYHT | 1 |
| | SRLHSGVPSRFSGSGSGTDYSLTISNLEQEDIATYFCQQGNTLPYTFGGGT | |
| | KLEITGSTSGSGKPGSGEGSTKGEVKLQESGPGLVAPSQSLSVTCTVSGVS | |
| | LPDYGVSWIRQPPRKGLEWLGVIWGSETTYYNSALKSRLTIIKDNSKSQVF | |
| | LKMNSLQTDDTAIYYCAKHYYYGGSYAMDYWGQGTSVTVSSAAAIEVMYPP | |
| | PYLDNEKSNGTIIHVKGKHLCPSPLFPGPSKPFWVLVVVGGVLACYSLLVT | |
| | VAFIIFWVRSKRSRLLHSDYMNMTPRRPGPTRKHYQPYAPPRDFAAYRSRV | |
| | KFSRSADAPAYQQGQNQLYNELNLGRREEYDVLDKRRGRDPEMGGKPRRKN | |
| | PQEGLYNELQKDKMAEAYSEIGMKGERRRGKGHDGLYQGLSTATKDTYDAL | |
| | HMQALPPR | |
| Anti-CD19 | DIQMTQTTSSLSASLGDRVTISCRASQDISKYLNWYQQKPDGTVKLLIYHT | 5 |
| (FMC63) VL | SRLHSGVPSRFSGSGSGTDYSLTISNLEQEDIATYFCQQGNTLPYTFGGGT | |
| | KLEIT | |
| Whitlow linker | GSTSGSGKPGSGEGSTKG | 6 |
| Anti-CD19 | EVKLQESGPGLVAPSQSLSVTCTVSGVSLPDYGVSWIRQPPRKGLEWLGVI | 4 |
| (FMC63) VH | WGSETTYYNSALKSRLTIIKDNSKSQVFLKMNSLQTDDTAIYYCAKHYYYG | |
| | GSYAMDYWGQGTSVTVSS | |
| Anti-CD19 scFv | DIQMTQTTSSLSASLGDRVTISCRASQDISKYLNWYQQKPDGTVKLLIYHT | 7 |
| | SRLHSGVPSRFSGSGSGTDYSLTISNLEQEDIATYFCQQGNTLPYTFGGGT | |
| | KLEITGSTSGSGKPGSGEGSTKGEVKLQESGPGLVAPSQSLSVTCTVSGVS | |
| | LPDYGVSWIRQPPRKGLEWLGVIWGSETTYYNSALKSRLTIIKDNSKSQVF | |
| | LKMNSLQTDDTAIYYCAKHYYYGGSYAMDYWGQGTSVTVSS | |
| Anti-CD19 VH | DYGVS | 21 |
| CDR1 | | |
| Anti-CD19 VH | VIWGSETTYYNSALKS | 22 |
| CDR2 | | |
| Anti-CD19 VH | HYYYGGSYAMDY | 23 |
| CDR3 | | |
| Anti-CD19 VL | RASQDISKYLN | 24 |
| CDR1 | | |
| Anti-CD19 VL | HTSRLHS | 25 |
| CDR2 | | |
| Anti-CD19 VL | QQGNTLPYT | 26 |
| CDR3 | | |
| Spacer | AAA | |
| CD28 hinge | IEVMYPPPYLDNEKSNGTIIHVKGKHLCPSPLFPGPSKP | 8 |
| CD28 | FWVLVVVGGVLACYSLLVTVAFIIFWV | 9 |
| transmembrane | | |
| domain | | |
| CD28 | RSKRSRLLHSDYMNMTPRRPGPTRKHYQPYAPPRDFAAYRS | 10 |
| intracellular | | |
| domain | | |
| CD3 zeta | RVKFSRSADAPAYQQGQNQLYNELNLGRREEYDVLDKRRGRDPEMGGKPRR | 11 |
| | KNPQEGLYNELQKDKMAEAYSEIGMKGERRRGKGHDGLYQGLSTATKDTYD | |
| | ALHMQALPPR | |
| Anti-CD20 CAR | QVQLVQSGAEVKKPGASVKVSCKASGYTFKEYGISWVRQAPGQGLEWMGWI | 2 |
| | SAYSGHTYYAQKLQGRVTMTTDTSTSTAYMELRSLRSDDTAVYYCARGPHY | |
| | DDWSGFIIWFDPWGQGTLVTVSSGSTSGSGKPGSGEGSTKGDIQMTQSPSS | |
| | LSASVGDRVTITCRASQSISSYLNWYQQKPGKAPKLLIYAASSLQSGVPSR | |
| | FSGSGSGTDFTLTISSLQPEDFATYYCQQSYRFPPTFGQGTKVEIKAAAFV | |
| | PVFLPAKPTTTPAPRPPTPAPTIASQPLSLRPEACRPAAGGAVHTRGLDFA | |
| | CDIYIWAPLAGTCGVLLLSLVITLYCNHRNRFSVVKRGRKKLLYIFKQPFM | |
| | RPVQTTQEEDGCSCRFPEEEEGGCELRVKFSRSADAPAYQQGQNQLYNELN | |
| | LGRREEYDVLDKRRGRDPEMGGKPRRKNPQEGLYNELQKDKMAEAYSEIGM | |
| | KGERRRGKGHDGLYQGLSTATKDTYDALHMQALPPR | |
| Anti-CD20 | QVQLVQSGAEVKKPGASVKVSCKASGYTFKEYGISWVRQAPGQGLEWMGWI | 12 |
| (Ab4) VH | SAYSGHTYYAQKLQGRVTMTTDTSTSTAYMELRSLRSDDTAVYYCARGPHY | |
| | DDWSGFIIWFDPWGQGTLVTVSS | |
| Whitlow linker | GSTSGSGKPGSGEGSTKG | 6 |
| Anti-CD20 | DIQMTQSPSSLSASVGDRVTITCRASQSISSYLNWYQQKPGKAPKLLIYAA | 13 |
| (Ab4) VL | SSLQSGVPSRFSGSGSGTDFTLTISSLQPEDFATYYCQQSYRFPPTFGQGT | |
| | KVEIK | |
| Anti-CD20 scFv | QVQLVQSGAEVKKPGASVKVSCKASGYTFKEYGISWVRQAPGQGLEWMGWI | 14 |
| | SAYSGHTYYAQKLQGRVTMTTDTSTSTAYMELRSLRSDDTAVYYCARGPHY | |
| | DDWSGFIIWFDPWGQGTLVTVSSGSTSGSGKPGSGEGSTKGDIQMTQSPSS | |
| | LSASVGDRVTITCRASQSISSYLNWYQQKPGKAPKLLIYAASSLQSGVPSR | |
| | FSGSGSGTDFTLTISSLQPEDFATYYCQQSYRFPPTFGQGTKVEIK | |
| Anti-CD20 VH | EYGIS | 27 |
| CDR1 | | |
| Anti-CD20 VH | WISAYSGHTYYAQKLQ | 28 |
| CDR2 | | |
| Anti-CD20 VH | GPHYDDWSGFIIWFDP | 29 |
| CDR3 | | |
| Anti-CD20 VL | RASQSISSYLN | 30 |
| CDR1 | | |
| Anti-CD20 VL | AASSLQS | 31 |
| CDR2 | | |
| Anti-CD20 VL | QQSYRFPPT | 32 |
| CDR3 | | |
| Spacer | AAA | |
| CD8 hinge | FVPVFLPAKPTTTPAPRPPTPAPTIASQPLSLRPEACRPAAGGAVHTRGLD | 15 |
| | FACDIY | |
| CD8 | IWAPLAGTCGVLLLSLVITLYCNHRN | 16 |
| transmembrane | | |
| domain | | |
| 4-1BB co- | RFSVVKRGRKKLLYIFKQPFMRPVQTTQEEDGCSCRFPEEEEGGCEL | 17 |
| stimulatory | | |
| domain | | |
| CD3 zeta | RVKFSRSADAPAYQQGQNQLYNELNLGRREEYDVLDKRRGRDPEMGGKPRR | 11 |
| | KNPQEGLYNELQKDKMAEAYSEIGMKGERRRGKGHDGLYQGLSTATKDTYD | |
| | ALHMQALPPR | |
| Bicistronic CAR | MLLLVTSLLLCELPHPAFLLIPDIQMTQTTSSLSASLGDRVTISCRASQDI | 3 |
| (in KITE-001) | SKYLNWYQQKPDGTVKLLIYHTSRLHSGVPSRFSGSGSGTDYSLTISNLEQ | |
| | EDIATYFCQQGNTLPYTFGGGTKLEITGSTSGSGKPGSGEGSTKGEVKLQE | |
| | SGPGLVAPSQSLSVTCTVSGVSLPDYGVSWIRQPPRKGLEWLGVIWGSETT | |
| | YYNSALKSRLTIIKDNSKSQVFLKMNSLQTDDTAIYYCAKHYYYGGSYAMD | |
| | YWGQGTSVTVSSAAAIEVMYPPPYLDNEKSNGTIIHVKGKHLCPSPLFPGP | |
| | SKPFWVLVVVGGVLACYSLLVTVAFIIFWVRSKRSRLLHSDYMNMTPRRPG | |
| | PTRKHYQPYAPPRDFAAYRSRVKFSRSADAPAYQQGQNQLYNELNLGRREE | |
| | YDVLDKRRGRDPEMGGKPRRKNPQEGLYNELQKDKMAEAYSEIGMKGERRR | |
| | GKGHDGLYQGLSTATKDTYDALHMQALPPRRAKRSGSGEGRGSLLTCGDVE | |
| | ENPGPMALPVTALLLPLALLLHAARPQVQLVQSGAEVKKPGASVKVSCKAS | |
| | GYTFKEYGISWVRQAPGQGLEWMGWISAYSGHTYYAQKLQGRVTMTTDTST | |
| | STAYMELRSLRSDDTAVYYCARGPHYDDWSGFIIWFDPWGQGTLVTVSSGS | |
| | TSGSGKPGSGEGSTKGDIQMTQSPSSLSASVGDRVTITCRASQSISSYLNW | |
| | YQQKPGKAPKLLIYAASSLQSGVPSRFSGSGSGTDFTLTISSLQPEDFATY | |
| | YCQQSYRFPPTFGQGTKVEIKAAAFVPVFLPAKPTTTPAPRPPTPAPTIAS | |
| | QPLSLRPEACRPAAGGAVHTRGLDFACDIYIWAPLAGTCGVLLLSLVITLY | |
| | CNHRNRFSVVKRGRKKLLYIFKQPFMRPVQTTQEEDGCSCRFPEEEEGGCE | |
| | LRVKFSRSADAPAYQQGQNQLYNELNLGRREEYDVLDKRRGRDPEMGGKPR | |
| | RKNPQEGLYNELQKDKMAEAYSEIGMKGERRRGKGHDGLYQGLSTATKDTY | |
| | DALHMQALPPR | |
| GMCSFRα | MLLLVTSLLLCELPHPAFLLIP | 18 |
| signal | | |
| Furin-GSG-T2A | RAKRSGSGEGRGSLLTCGDVEENPGP | 19 |
| CD8a signal | MALPVTALLLPLALLLHAARP | 20 |
| KITE-001 | | |
| nucleic acid | ATGCTGCTGCTGGTGACATCTCTGCTGCTTTGCGAGCTGCCCCACCCTGCC | 39 |
| sequence | TTCCTGCTTATCCCCGACATTCAGATGACCCAGACCACCAGCAGCCTGAGC | |
| | GCCAGCTTAGGAGATAGAGTTACCATCAGCTGCAGAGCCAGCCAGGACATC | |
| | AGCAAATACCTGAACTGGTATCAGCAGAAGCCCGACGGCACTGTGAAACTG | |
| | CTTATTTACCACACCTCCAGACTGCACAGCGGCGTTCCCAGCAGATTCTCT | |
| | GGCAGCGGATCTGGAACCGACTACAGCCTCACCATCTCCAACCTGGAGCAG | |
| | GAGGACATCGCCACCTACTTCTGCCAGCAGGGCAACACACTGCCCTACACC | |
| | TTCGGAGGAGGAACCAAGCTGGAGATCACCGGCTCCACCTCTGGATCCGGC | |
| | AAGCCCGGATCTGGCGAGGGATCCACCAAGGGCGAGGTTAAGCTGCAGGAG | |
| | AGCGGCCCTGGCCTGGTGGCTCCTAGCCAATCTTTATCTGTGACCTGCACT | |
| | GTGTCCGGCGTTAGCCTGCCCGATTATGGCGTTTCCTGGATCAGACAGCCC | |
| | CCCAGAAAGGGCCTGGAATGGCTGGGCGTTATCTGGGGCAGCGAGACCACA | |
| | TACTACAACAGCGCCCTGAAGAGCAGACTTACGATTATCAAGGACAACAGC | |
| | AAGAGCCAGGTTTTCCTGAAGATGAACAGCCTGCAGACCGACGACACCGCC | |
| | ATCTACTACTGCGCTAAGCACTACTACTACGGCGGCAGCTACGCCATGGAC | |
| | TACTGGGGCCAGGGAACAAGCGTTACCGTTAGCAGCGCTGCTGCAATTGAA | |
| | GTTATGTATCCTCCTCCTTACCTGGACAACGAGAAGAGCAACGGCACCATC | |
| | ATCCACGTTAAGGGCAAGCACCTGTGCCCCAGCCCTCTGTTCCCTGGACCT | |
| | TCTAAGCCTTTCTGGGTTCTGGTGGTGGTCGGCGGCGTTTTAGCCTGTTAC | |
| | AGCCTTCTGGTGACTGTGGCCTTCATCATCTTTTGGGTTAGAAGCAAGAGA | |
| | AGCAGACTGCTCCACAGCGACTACATGAACATGACCCCCAGACGGCCTGGC | |
| | CCCACCAGAAAGCATTACCAGCCCTACGCTCCTCCCAGAGACTTCGCCGCC | |
| | TACAGGAGCAGAGTTAAATTCAGCAGATCCGCCGATGCCCCCGCTTACCAA | |
| | CAGGGACAAAACCAGCTGTACAATGAGCTCAACCTGGGGAGAAGAGAAGAA | |
| | TACGACGTTCTGGATAAGAGAAGGGGCAGAGATCCCGAAATGGGGGGCAAG | |
| | CCCAGACGCAAGAACCCTCAGGAGGGGCTTTACAACGAACTGCAGAAGGAT | |
| | AAGATGGCTGAGGCTTACTCGGAGATTGGGATGAAGGGGGAGAGAAGGCGG | |
| | GGCAAGGGACACGATGGCTTATACCAGGGGCTGAGCACCGCCACCAAGGAC | |
| | ACATACGACGCTCTTCATATGCAGGCTCTGCCCCCAAGAAGGGCTAAGAGA | |
| | TCTGGCTCTGGCGAGGGCAGAGGCAGCTTGCTTACATGTGGCGATGTGGAG | |
| | GAGAACCCCGGGCCCATGGCTCTTCCTGTGACAGCTCTTCTGCTGCCCCTG | |
| | GCCCTGCTTCTGCATGCTGCTAGACCTCAGGTTCAGCTTGTGCAGAGCGGA | |
| | GCTGAAGTTAAGAAGCCTGGCGCCTCTGTGAAGGTTAGCTGCAAGGCCAGC | |
| | GGCTACACATTCAAGGAATATGGCATCTCCTGGGTTAGGCAGGCTCCCGGC | |
| | CAAGGCTTAGAATGGATGGGCTGGATCTCCGCCTACTCCGGCCACACCTAC | |
| | TACGCCCAGAAGCTTCAGGGCAGGGTTACCATGACCACCGACACCAGCACC | |
| | TCTACCGCCTATATGGAGCTGAGGAGCCTGAGATCGGACGACACAGCTGTG | |
| | TATTACTGCGCCAGAGGCCCCCACTACGACGACTGGTCTGGATTTATCATC | |
| | TGGTTCGACCCCTGGGGGCAGGGCACCCTGGTCACAGTTTCTTCTGGCTCC | |
| | ACCAGCGGAAGCGGCAAGCCAGGCTCAGGCGAAGGATCTACAAAAGGCGAC | |
| | ATCCAAATGACACAGAGCCCCAGCAGCTTGAGCGCCTCCGTTGGCGACAGA | |
| | GTTACAATCACCTGCAGGGCCTCTCAGAGCATCAGCAGCTATTTGAATTGG | |
| | TATCAACAGAAGCCAGGAAAGGCCCCTAAGCTGCTCATCTACGCTGCCAGC | |
| | TCGCTCCAATCTGGCGTTCCTAGCAGATTTAGCGGCTCCGGCAGCGGCACA | |
| | GACTTTACTCTTACCATTAGCTCCCTGCAGCCCGAGGACTTCGCTACCTAC | |
| | TATTGCCAGCAAAGCTACAGATTCCCTCCCACCTTTGGCCAGGGCACAAAG | |
| | GTTGAGATCAAGGCAGCTGCTTTCGTGCCTGTGTTCCTGCCTGCTAAGCCC | |
| | ACCACCACTCCTGCTCCAAGACCTCCTACCCCCGCTCCTACAATCGCCAGC | |
| | CAACCTCTGAGCCTGAGACCGGAGGCATGCAGACCTGCGGCAGGGGGAGCA | |
| | GTTCACACAAGAGGCTTGGACTTCGCTTGCGACATCTACATCTGGGCCCCT | |
| | CTGGCCGGCACATGCGGAGTTCTTCTTCTTAGCCTGGTGATCACCCTGTAC | |
| | TGCAACCACAGAAACAGATTCAGCGTTGTGAAGAGAGGCCGGAAGAAGCTG | |
| | CTGTACATCTTCAAGCAGCCCTTCATGAGACCTGTGCAGACCACACAGGAG | |
| | GAAGACGGCTGCAGCTGTAGATTCCCCGAGGAAGAGGAGGGCGGCTGTGAG | |
| | CTGAGAGTTAAGTTCAGCAGGAGCGCCGACGCCCCTGCCTACCAGCAAGGA | |
| | CAGAATCAACTGTACAACGAGCTGAACCTGGGCAGACGGGAGGAATACGAT | |
| | GTGCTGGACAAGAGGAGAGGCAGAGACCCCGAGATGGGCGGCAAACCTAGA | |
| | AGAAAGAACCCCCAGGAGGGCCTGTATAACGAGCTCCAGAAGGACAAGATG | |
| | GCCGAGGCCTACAGCGAGATCGGCATGAAGGGCGAAAGAAGAAGAGGCAAG | |
| | GGCCACGACGGCCTCTACCAGGGCTTAAGCACAGCTACAAAGGACACCTAC | |
| | GACGCCCTGCACATGCAGGCCCTGCCCCCTAGATGA | |

### Generation of KITE-001 for In Vitro and In Vivo Characterization

Anti-CD19/CD20 CAR T cells were generated from T cells isolated from apheresis material from 3 healthy human donors transduced with an LVV encoding the anti-CD19/CD20 CAR construct.

Anti-CD19/CD20 CAR T cells were also generated from T cells isolated from apheresis material from patients with autoimmune diseases: 2 patients with SLE without LN (SLE-248 and SLE-254), 2 patients with LN (LN-972 and LN-168), and 2 patients with SSc (SSc-430 and SSc-589).

### Detection of Dual and Total CAR Expression in KITE-001 T-Cell Products

Cell-surface expression of both the anti-CD19 CAR and anti-CD20 CAR was individually detected using anti-idiotypic antibodies, and total CAR expression was also determined. In KITE-001 products generated from apheresis material collected from 3 healthy human donors, anti-CD19 CAR expression ranged from 29% to 64%, anti-CD20 CAR expression ranged from 42% to 79%, and total CAR (CD19 and/or CD20) expression ranged from 45% to 82%.

Cell-surface expression of both the anti-CD19 CAR and anti-CD20 CAR as well as total CAR expression was also determined in KITE-001 products generated from apheresis material collected from patients with autoimmune diseases. Given the consistency of data observed across the various autoimmune indications tested, data from KITE-001 products generated from patients with SLE without LN (SLE-248 and SLE-254) are summarized as a representative example. Individual expression of the anti-CD19 CAR was 76% for SLE-248 and 53% for SLE-254, and 75% and 53% respectively, for the anti-CD20 CAR in the same samples. Total CAR expression of KITE-001 products, as measured by the sum of total anti-CD19 and anti-CD20 CAR expression, was 79% and 57% for SLE-248 and SLE-254, respectively. In contrast, NTD control T cell samples did not stain positively for either anti-CD19 or anti-CD20 CAR expression.

### Functional Characterization of Anti-CD19/CD20 CAR T Cells

The in vitro functional activity of the anti-CD19/CD20 CAR T-cell (KITE-001) products generated from apheresis material from 3 healthy human donors was demonstrated by the production of pro-inflammatory cytokines and cytolytic activity after engagement with CD19/CD20 antigen-expressing target cells, regardless of whether one or both antigens were present. For these studies, a B-cell lymphoma cell line (Raji) and a B-cell acute lymphoblastic leukemia cell line (Nalm6) were used; both (wild type [WT]) cell lines express CD19 and CD20 antigens. To determine the specificity of the CAR T-cell product binding to either CD19 or CD20, the cell lines were also engineered to express only 1 or the other antigen (i.e., CD19 KO cells expressed only CD20, and CD20 KO cells expressed only CD19). A double-antigen KO (DKO) negative control Raji cell line (CD19⁻CD20⁻) was engineered by knocking out CD19 and CD20.

Similarly, the in vitro functional activity of the KITE-001 products generated from apheresis material from patients with autoimmune diseases was demonstrated by T-cell proliferation, production of pro-inflammatory cytokines, and cytolytic activity after co-culture with CD19-expressing and CD20-expressing target cells. Given the consistency of data observed across the various autoimmune indications tested, data from KITE-001 products generated from patients with SLE without LN (SLE-248 and SLE-254) are summarized below as a representative sample. For these studies, a B-cell lymphoma cell line (Raji) and an antigen-negative (CD19⁻CD20⁻) K-562 cell line (chronic myelogenous leukemia) was used to further evaluate CAR specificity.

Antigen-Driven Proliferation of KITE-001 Products from Healthy Donors

Anti-CD19/CD20 CAR T-cell activation was measured by flow cytometry using an anti-4-1BB antibody (4-1BB is induced when T cells are activated upon engagement with antigen). KITE-001 products showed robust proliferation after 4 days of co-culture with dual antigen-expressing Raji WT and Nalm6 WT target cells and single antigen-expressing Raji CD19KO or Raji CD20 KO cells, as shown by flow cytometry, demonstrating that both the anti-CD19 CAR and the anti-CD20 CAR in the KITE-001 products are functional. In comparison, the nontransduced control T cells showed only basal, homeostatic levels of proliferation in the presence of different target cell lines, and minimal proliferation was observed when T-cell products were cultured in the absence of target cells.

Antigen-Driven Proliferation of KITE-001 Products from Patients with SLE

To assess antigen-specific proliferation of the CAR T-cell products from patients with SLE upon engagement with target cells, KITE-001 and NTD products that had been co-cultured with Raji, Raji CD19KO, Raji CD20KO cells, K-562, and autologous B cells were harvested 4 days after initiation of co-culture. T cells had been pre-labeled with CellTrace™ Violet, a fluorescent dye used to trace multiple generations of cells by analysis of dye dilution upon cell division. T-cell product co-cultured with target cell lines at an E:T ratio of 1:1 was used whereas autologous B cells at an E:T ratio of 1:1.5 for SLE-248 or 1:2 for SLE-254 was used to match the number of T-cell product seeded for all conditions. T-cell products that had been cultured in the absence of target cells were also used as controls (T cells alone). Upon harvest, cells were stained with fluorescently-labelled antibodies against CD3, CD4, and CD8 to discriminate T cells from target cells, as well as a viability dye.

KITE-001 products from patients with SLE showed robust proliferation against Raji, Raji CD19KO, and Raji CD20KO cells, demonstrating that both the anti-CD19 CAR and the anti-CD20 CAR in KITE-001 products from patients with SLE were functional. Importantly, KITE-001 products from patients with SLE exhibited robust proliferation in response to autologous B cells. KITE-001 products displayed minimal proliferation against antigen-negative K-562 cells, indicating target-specific proliferation. Furthermore, NTD cells displayed minimal to no non-specific proliferation across all co-culture conditions, and T cells cultured alone showed no proliferation. Overall, these results demonstrate that KITE-001 products from patients with SLE showed robust antigen-dependent and -specific proliferation from co-culture with antigen-positive target cells, including autologous B cells.

Antigen-Driven Cytokine Production by KITE-001 Products from Heathy Donors

The anti-CD19/CD20 CAR T cells (KITE-001) were tested for functionality by assessing the production of pro-inflammatory cytokines upon stimulation with antigen-positive target cells. Briefly, on Day 1 after co-culture initiation, supernatants were collected and levels of pro-inflammatory cytokines were assayed via a panel kit. Two multiplicities of infection (MOI; MOI=5 and MOI=10) were used to understand the relationship between the number of infectious viral particles per cell and transduction efficiency. Supernatants from the co-cultures of T-cell products plated at the 1:1 effector-to-target (E:T) ratio with dual or single antigen-expressing target cells (Nalm6 WT, Raji WT, Raji CD19KO, and Raji CD20KO) were analyzed for levels of interferon (IFN)-7, IL-2, and tumor necrosis factor alpha (TNF)-α secretion mediated by antigen engagement. Supernatants from T cells co-cultured with antigen-negative Raji DKO target cells and from T cells cultured in the absence of target cells (T cells alone) were analyzed in parallel to assess antigen-independent allogeneic-driven cytokine production or basal levels of cytokine production in the absence of antigen, respectively. All samples were diluted to be within the range of detection.

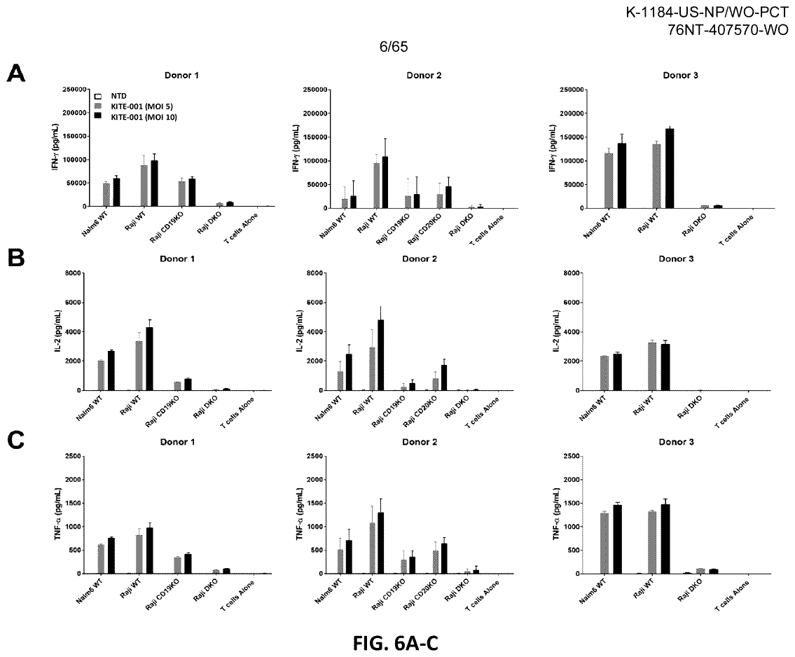

The anti-CD19/CD20 CAR T cells demonstrated functionality by the production of the pro-inflammatory cytokines IFN-7, IL-2, and TNF-α after a 1-day co-culture with dual antigen-positive Nalm6 WT and Raji WT target cells. In contrast, nontransduced control T cells did not produce detectable cytokines when co-cultured with the same target cells (FIG. 6A-C). This observation was consistent for KITE-001 products generated from T cells from each of the 3 donors. The functionality of the anti-CD19 CAR and anti-CD20 CAR in the KITE-001 products was also demonstrated separately by the production of IFN-7, IL-2, and TNF-α after 1 day of co-culture with single-antigen positive Raji CD20 KO (CD19⁺CD20⁻) target cells and Raji CD19 KO (CD19⁻CD20⁺) target cells, respectively, albeit at levels lower than those induced by co-culture with dual-antigen positive Raji WT (CD19⁺CD20⁺) target cells. In contrast, minimal or no cytokine release was observed when anti-CD19/CD20 CAR T cells were co-cultured with Raji DKO (CD19⁻CD20⁻) target cells, or in the absence of target cells (T cells alone). Production of comparable levels of IFN-γ, IL-2, and TNF-α were observed from KITE-001 products regardless of the MOI used for their generation. These results indicate that the KITE-001 products produced pro-inflammatory cytokines when stimulated with antigen-positive target cells.

Antigen-Driven Cytokine Production by KITE-001 Products from Patients with SLE

Specific product functionality was further evaluated by measurement of cytokine levels in culture supernatants after an overnight co-culture of KITE-001 products from patients with SLE with Raji, Raji CD19KO, Raji CD20KO, and K-562 cells at an E:T ratio of 1:1 (25,000 T cells: 25,000 target cells) and with autologous B cells at 1:1.5 for SLE-248 and 1:2 for SLE-254 in which the number of product T cells seeded is equivalent to 25,000 T cells in all co-culture conditions. Quantitation of IFN-γ, interleukin-2 (IL-2), and TNF-α was performed using the Mesoscale Discovery (MSD) platform.

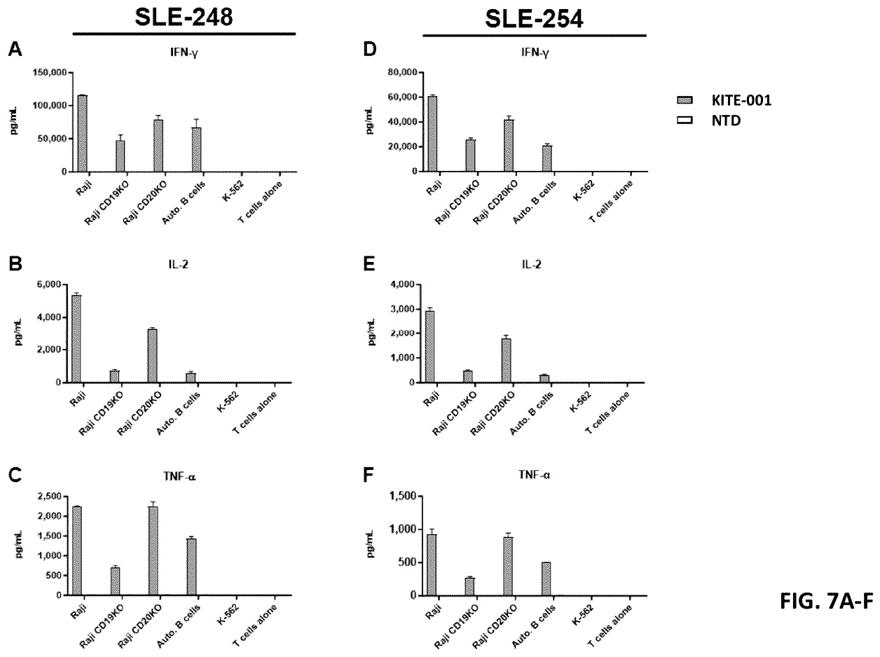

FIG. 7 shows specific cytokine production from co-culture of KITE-001 products with the antigen-positive Raji cells (CD19⁺CD20⁺). The functionality of each individual CAR was demonstrated by the production of cytokines when the KITE-001 products were co-cultured with target cells expressing a single antigen (Raji CD19KO or Raji CD20KO), albeit at lower levels than that induced by co-culture with Raji parental cells, which express both antigens. Importantly, KITE-001 products from patients with SLE exhibited cytokine production in response to autologous B cells. In contrast, cytokine release was not observed in all NTD T-cell co-culture conditions and when KITE-001 products were co-cultured with antigen-negative K-562 target cells or in the absence of target cells (T cells alone). These results demonstrate that KITE-001 products from patients with SLE showed antigen-dependent and -specific production of IFN-γ, IL-2, and TNF-α in co-culture with target cells, including autologous B cells.

### Cytotoxicity Against Target Cells of KITE-001 Products From Healthy Donors

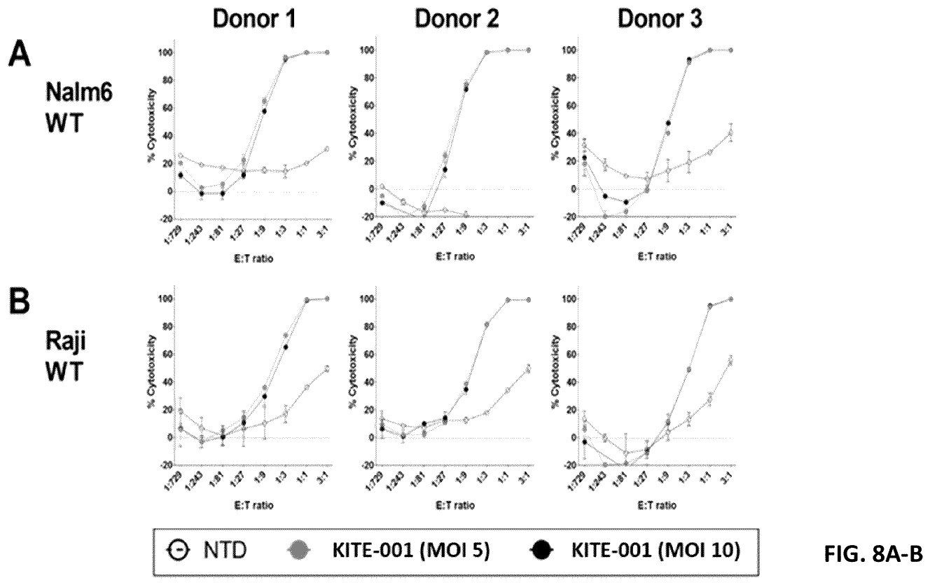

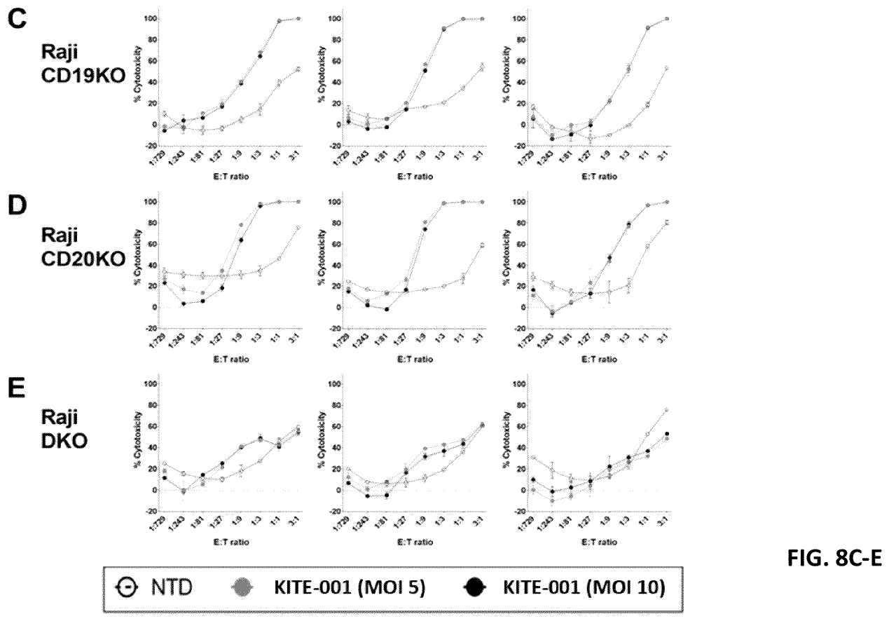

The anti-CD19/CD20 CAR T-cell (KITE-001) products demonstrated dose-dependent cytotoxicity against dual antigen-positive (i.e., CD19⁺CD20⁺) target cells, Nalm6 WT (FIG. 8A) and Raji WT (FIG. 8B), as well as against single antigen-positive target cells, Raji CD19 KO (FIG. 8C) and Raji CD20 KO (FIG. 8D). T-cell mediated cytotoxicity was measured after 1 and 4 days of co-culture with luciferase-labeled target cells as a function of the reduction in target luciferase signal in co-culture wells compared with the signal emitted by target cells plated alone. The degree of cytotoxicity against single antigen-positive target cells at Day 4, Raji CD19 KO (FIG. 8C) and Raji CD20 KO (FIG. 8D), was comparable with that shown against dual antigen-positive Raji WT cells (FIG. 8B), demonstrating that both the anti-CD19 CAR and anti-CD20 CAR were independently functional.

KITE-001 showed cytotoxic activity against all antigen-positive target cells in an E:T-dependent (FIGS. 8A-D) and an antigen-specific manner (FIG. 8E), as shown by minimal cytotoxicity against Raji DKO cells that do not express either CD19 or CD20 antigens. Basal levels of antigen-independent cytotoxic activity mediated by alloreactivity against all target cells was observed for T-cell products, as shown by the CAR-independent cytotoxicity mediated by the nontransduced control T cells, and by the activity of all T-cell products, regardless of CAR expression, against the negative control target cells, Raji DKO, which do not express either CD19 or CD20 antigens. Antigen-independent cytotoxic activity was dose-dependent and was more evident at the higher E:T ratios. Results were comparable at both MOIs and across KITE-001 products.

Cytotoxicity Against Target Cells of KITE-001 Products from Patients with SLE

The functionality of the KITE-001 products from patients with SLE were assessed in co-culture assays with luciferase-expressing target cells or patient-derived negative fraction cells containing autologous primary B cells. The negative fraction consists of the remaining white blood cells (from leukopaks) after T cells had been isolated to generate the CAR T-cell products. For cell lines, cytotoxicity was measured as the reduction of the luciferase signal emitted in the test wells upon addition of luciferin compared with the wells where target cells alone were seeded.

Multiple E:T ratios were used in these assays ranging from 3:1 to 1:81, with the number of targets held constant at 25,000 cells. Two cell lines were used: Raji, a B-cell lymphoma cell line that expresses high levels of CD19 and CD20 antigens; and K-562, a chronic myelogenous leukemia cell line that does not express CD19 or CD20 antigens. To independently assess the activity of the anti-CD19 and anti-CD20 CARs, Raji CD19KO and Raji CD20KO cells were used. Raji cell lines were engineered using clustered regularly interspaced short palindromic repeats (CRISPR) technology and clonally selected to express either antigen: Raji CD19KO cells only express CD20 and not CD19, while Raji CD20KO only express CD19 and not CD20. Antigen KO was confirmed at the DNA level by tracking of insertions or deletions (indels) by decomposition (TIDE) analysis and at the cell-surface protein level by flow cytometry.

For unlabeled autologous B cells, cytotoxicity was measured via flow cytometry by the reduction of absolute B cell counts in co-culture wells compared with B cells seeded alone. Since the target primary B cells constitutes only a portion of cells within the negative fraction, 125,000 negative fraction cells were seeded rendering the E:T ratios effectively at 2:1, 1:1.5, and 1:4.5 for patient SLE-248, and 1.5:1, 1:2, and 1:6 for patient SLE-254 respectively. NTD T-cell products were included in the co-culture experiments as controls to assess levels of alloreactivity. Additionally, CAR T-cell products cultured in the absence of any target cells were used to assess basal levels of T-cell function.

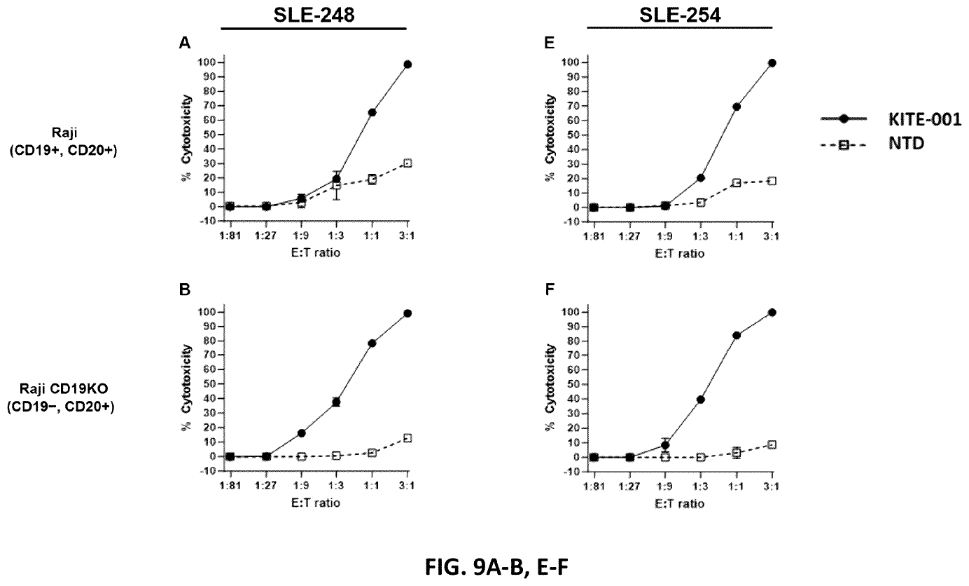

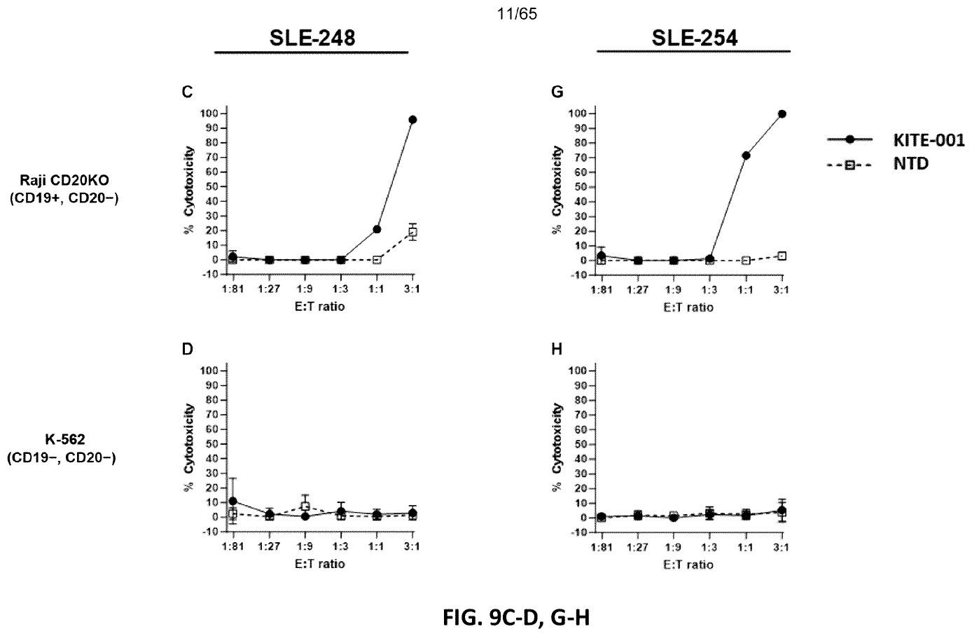

Through luciferase-based killing assay, KITE-001 products from 2 patients with SLE showed comparable dose-dependent specific cytotoxicity against CD19 and/or CD20-positive target cell lines on Day 4 of co-culture, regardless of expression of both or either antigen (FIG. 9). In contrast, control NTD T cells showed minimal to no cytotoxicity. KITE-001 products also displayed minimal to no cytotoxicity against antigen-negative K-562 cells, demonstrating target-specific cytotoxicity. Since the autologous B cells in the negative fraction were not luciferase-labeled, cytotoxicity against autologous B cells was assessed by flow cytometry.

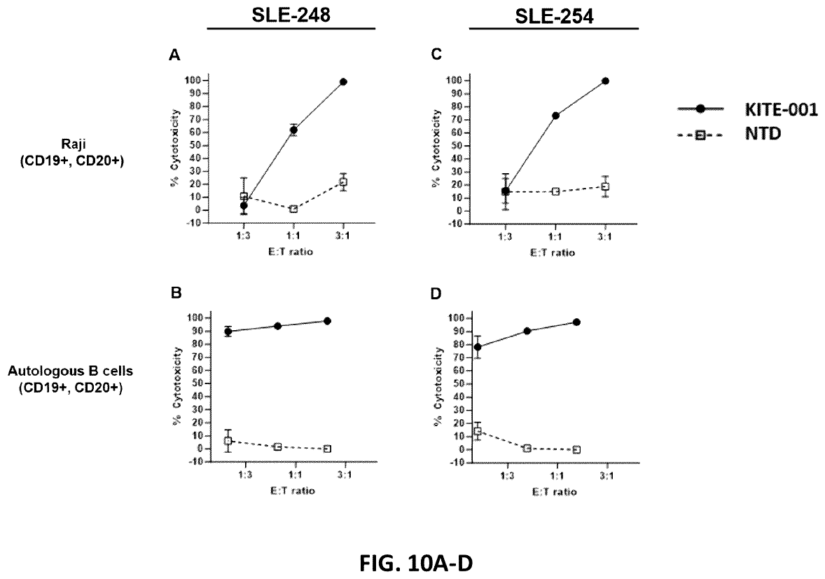

As demonstrated in FIG. 10, KITE-001 products exhibited dose-dependent cytolytic activity against Raji cells or autologous B cells at various E:T ratios, whereas no cytotoxicity was observed with NTD control cells. These results demonstrate that KITE-001 products from patients with SLE exhibit robust antigen-dependent and -specific cytotoxicity in co-culture with target cells, including autologous B cells.

### Example 6. Non-Clinical In Vivo Pharmacologic Evaluation of KITE-001

A xenograft mouse model of B-cell lymphoma using the Raji cell line in profoundly immune-deficient NSG mice was utilized in the in vivo KITE-001 efficacy study in this example.

### Efficacy Evaluation of KITE-001 Against a Disseminated Raji-Luc B-Cell Lymphoma Xenograft NSG Mouse Model

The antitumor activity of KITE-001 was evaluated in the xenograft mouse model of B-cell lymphoma. To establish the disseminated tumors, 5.0×10⁵ Raji-luc tumor cells were implanted IV via the lateral tail vein into 7- to 8-week-old female NSG mice (6 per group) on Day 0. On Day 6 after tumor cell implantation, nontransduced T cells or KITE-001 (5.0×10⁶ or 1.0×10⁶ CAR T cells) were administered IV. Animals receiving KITE-001 were supplemented with nontransduced T cells so that all test animals received the same total number of T cells (1.35×10⁷ total T cells). Beginning on Day 6 after tumor implantation, 36 g of human IL-2 was administered to each animal by intraperitoneal injection twice daily for 3 days.

### Antitumor Activity

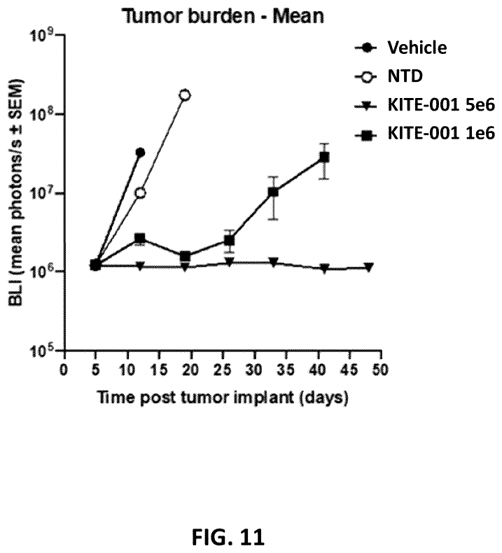

Antitumor activity was evaluated by tumor burden (measured by bioluminescence imaging) and tumor growth delay. A significant (p<0.05; analysis of variance with Tukey's post-test) and dose-dependent tumor growth inhibition was observed in the groups treated with KITE-001 when compared with the groups treated with the vehicle or nontransduced control T cells. The change in tumor burden (measured by bioluminescence and log₁₀ normalized) was assessed from Day 5 (1 day before treatment) to Day 12 (6 days after treatment and the last time point that all groups were intact). Tumor growth inhibition for the KITE-001-treated groups or the nontransduced T-cell control group when compared with tumor growth for the vehicle-treated group was 101.0% (KITE-001, 5×10⁶ CAR T cells), 83.1% (KITE-001, 1×10⁶ CAR T cells), and 35.9% (nontransduced T cells) (FIG. 11). The modest tumor growth inhibition caused by nontransduced T cells compared with vehicle is likely due to alloreactivity of the T cells and has been observed in these models across Kite studies. For mice treated with vehicle or nontransduced T cells, tumor burden increased rapidly as seen by body weight loss; the majority of animals were euthanized between Days 10 and 20 due to the effects of high tumor burden, as evidenced by body weight loss or adverse clinical signs.

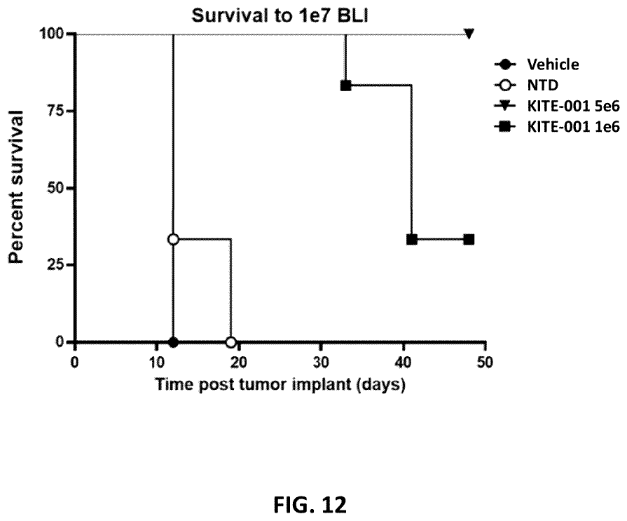

Treatment with KITE-001 also resulted in significant (p<0.05; log-rank [Mantel-Cox]test) dose-dependent tumor growth delay at both dose levels of KITE-001 compared with groups treated with vehicle or nontransduced T cells. The median survival time was not achieved by the study endpoint in the group treated with the higher dose (5×10⁶ CAR T cells) of KITE-001 compared with a median survival time of 41 days in the group treated with the lower dose of KITE-001 (1×10⁶ CAR T cells), and 12 days for the vehicle and nontransduced T-cell groups (FIG. 12).

### Body Weight

The higher dose of KITE-001 (5×10⁶ CAR T cells) was well tolerated with animals maintaining consistent body weight due to the significant control of tumor burden. The lower dose (1×10⁶ CAR T cells) was also well tolerated until body weight began to drop after Day 30, which coincided with this group exhibiting a loss of tumor burden control. Animals in the vehicle group had increased tumor burden resulting in a mean body weight loss of over 10% by Day 16, after which all animals were euthanized. Animals receiving nontransduced T cells showed a similar pattern of weight loss, with the exception of 1 animal that survived through the end of study; the animal did not reach the 20% body weight loss removal criteria, and exhibited evidence of some tumor burden control, reaching a plateau at a high tumor burden level, likely attributed to alloreactivity.

### CAR T-Cell Expansion and Kinetics

KITE-001 antitumor activity correlated with CAR T-cell expansion. Expansion of CAR T cells was observed in mice treated at both dose levels from 1 day (Day 7) to 7 days (Day 13) after injection of the CAR T cells, reaching peak expansion at Day 13. From Day 7 to Day 13, mice that received 5×10⁶ CAR T cells showed mean in vivo expansion to greater than 1,000 CAR T cells/L of murine blood and mice dosed with 1×10⁶ CAR T cells showed expansion to greater than 100 CAR T cells/L of murine blood, suggestive of a dose-dependent effect on peak expansion.

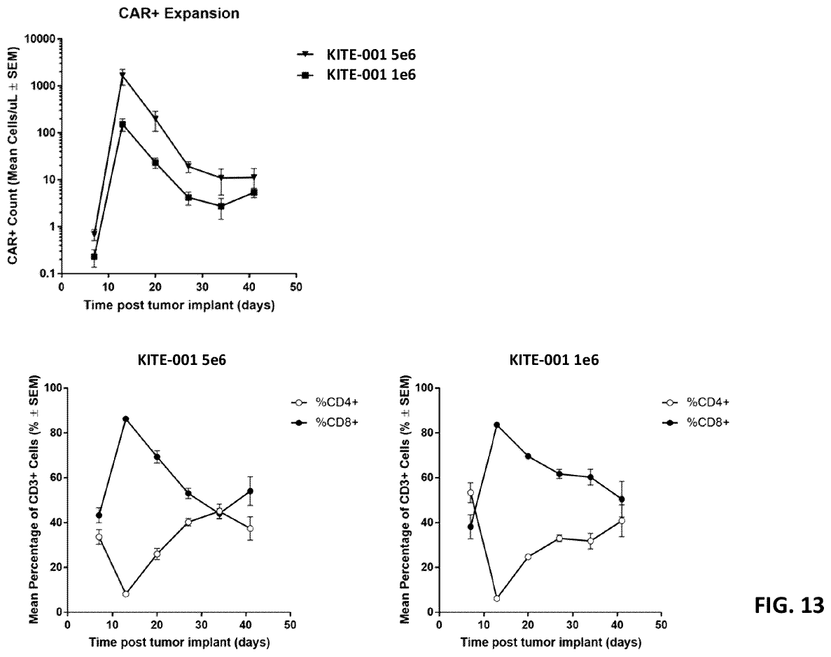

At peak expansion (Day 13) in this mouse model, CD8⁺ T-cell expansion reached 86% and 84% of total T cells (CD3⁺ cells) in mice treated with 5×10⁶ and 1×10⁶ CAR T cells, respectively (FIG. 13). Relative levels of CD4⁺ T cells concomitantly decreased to 8% and 6% of total T cells (CD3⁺ cells), respectively, on Day 13. After peak expansion, the CD4/CD8 ratio converged back to the original ratios observed at KITE-001 infusion on Day 6. Thus, anti-CD19/CD20 CAR T-cell expansion was driven primarily by CD8⁺ T-cell expansion in this mouse model (FIG. 13).

### Example 7. Clinical Study of KITE-001 in Cancer Patients

B-cell depletion is an expected outcome of treatment with KITE-001 and is the desired on-target pharmacodynamic effect of KITE-001. Herein, KITE-001-mediated B-cell depletion is summarized for a Phase 1, FIH study of KITE-001 in participants with r/r B-cell lymphomas.

A total of 3 participants were enrolled to dose level (DL)1 (0.5×10⁶ CAR T cells/kg), 8 participants enrolled to DL2 (1.0×10⁶ CAR T cells/kg), and 22 participants enrolled to DL3 (2.0×10⁶ CAR T cells/kg). CAR T-cell expansion was observed in blood of all participants with median peak on Days 10, 14, and 8 for DL1, DL2, and DL3, respectively.

At DL1 and DL2 (0.5×10⁶ and 1.0×10⁶ CAR T cells/kg, respectively), KITE-001 expanded to a similar median CAR T peak level as the 2.0×10⁶ CAR T cells/kg dose level of axicabtagene ciloleucel using a lymphodepleting chemotherapy regimen of CYC 500 mg/m² and fludarabine 30 mg/m² (data on file). Furthermore, at DL3 (2.0×10⁶ CAR T cells/kg), KITE-001 expanded to a 3-fold higher median CAR T-cell level (peak of 114 cells/μl blood) compared with participants treated with axicabtagene ciloleucel at the same dose level, suggesting an increased expansion potential of KITE-001 relative to axicabtagene ciloleucel.

Among all evaluated participants, 9 had not received prior CAR T-cell therapy and presented detectable B cells before infusion with KITE-001, which are 2 conditions needed to estimate the B-cell depletion effect of the KITE-001 product without possible confounding pre-existing B-cell lymphopenia or resistance to CAR T therapy.

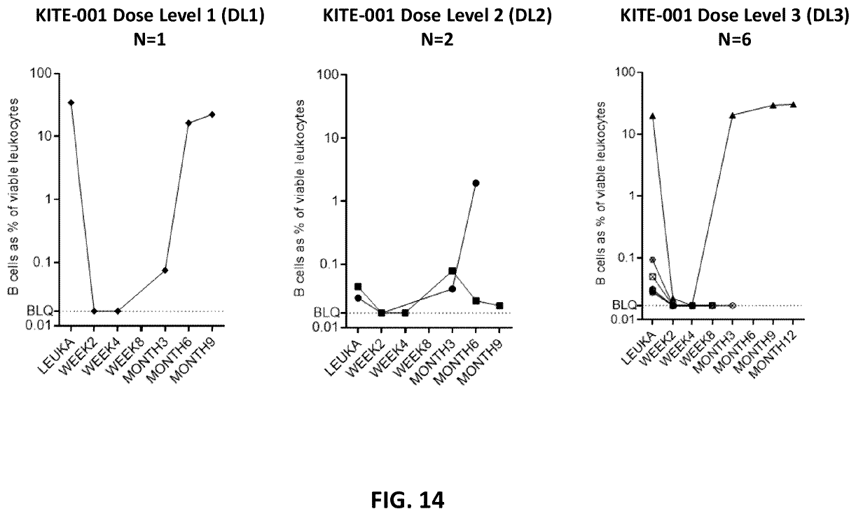

Within the first 2 to 4 weeks post KITE-001 treatment, all participants, at all 3 tested doses, presented complete B-cell depletion, defined as B-cell level ≤0.017% of PBMCs, where 0.017% is the flow cytometry assay lower limit of quantitation (FIG. 14). For all dose levels, B-cell recovery was observed in a participant-specific manner starting after Week 4 post-infusion when CAR T-cell levels decreased toward baseline levels.

### Clinical Safety

### Dose-Limiting Toxicities

As of the cutoff date for the B-cell aplasia data, no dose-limiting toxicities (DLTs) were reported.

### Treatment-Emergent Adverse Events

Treatment-emergent adverse events (TEAEs) are defined as any adverse event (AE) that begins on or after the date of the KITE-001 infusion. AEs are graded according to the National Cancer Institute (NCI) Common Terminology Criteria for Adverse Events (CTCAE) version 5.0 and coded according to the Medical Dictionary for Regulatory Activities (MedDRA) version 26.0. Cytokine release syndrome (CRS) is graded according to a modified CTCAE grading system and according to the American Society for Transplantation and Cellular Therapy (ASTCT) guidelines; however results reported herein are presented only for the Lee 2014 grading system. Neurologic events are graded according to CTCAE and ASTCT; however, results reported herein are per CTCAE as this is the grading system used to identify DLTs on this study.

An overview of TEAEs reported for the 33 participants dosed with KITE-001 in the study at the data cutoff date for this report is presented in Table 4.

All 33 participants (100%) treated with KITE-001 had at least 1 TEAE, including 22 participants (67%) with TEAEs Grade ≥3.

Of the 33 participants, 31 (94%) had TEAEs reported by the investigator as related to KITE-001, of which the majority were symptoms of CRS.

Fifteen participants experienced Grade ≥3 TEAEs related to KITE-001 that were not symptoms of CRS; events that occurred in more than 1 participant included: white blood cell count decreased (7 participants; worst Grade 3 to Grade 4), neutrophil count decreased (6 participants; worst Grade 3 to Grade 4), neutropenia (5 participants; worst Grade 3 to Grade 4, including 1 serious Grade 4 event), and encephalopathy (2 participants; worst Grade 2 to Grade 3, including 2 serious Grade 3 events). There were no Grade 5 events related to KITE-001.

Fifteen participants (45%) experienced at least 1 serious TEAE (SAE), of whom eleven (33%) had an SAE that was related to KITE-001. Five participants had KITE-001-related SAEs of serious CRS.

Eight participants experienced Grade ≥3 SAEs that were not considered related to KITE-001. One of these participants (Dose-escalation Cohort 2) had a fatal AE of myelodysplastic syndrome on Day 316 that was considered not related to KITE-001.

TABLE 4
Overall Summary of TEAEs

| | | Cohort 2 | Cohort 2 | Cohort 2 | | |
|---|---|---|---|---|---|---|
| | Cohort 1 | Escalation | Expansion | Overall | Cohort 3 | Overall |
| Participant Incidence, n (%) | (N = 3) | (N = 3) | (N = 5) | (N = 8) | (N = 22) | (N = 33) |
| Any TEAE | 3 (100) | 3 (100) | 5 (100) | 8 (100) | 22 (100) | 33 (100) |
| Any serious TEAE | 3 (100) | 1 (33) | 1 (20) | 2 (25) | 10 (45) | 15 (45) |
| Any KITE-001-related TEAE | 3 (100) | 3 (100) | 4 (80) | 7 (88) | 21 (95) | 31 (94) |
| Any KITE-001-related serious TEAE | 2 (67) | 1 (33) | 1 (20) | 2 (25) | 7 (32) | 11 (33) |
| Grade ≥3 TEAE | 2 (67) | 3 (100) | 2 (40) | 5 (63) | 15 (68) | 22 (67) |
| Grade ≥3 KITE-001-related TEAE | 2 (67) | 2 (67) | 2 (40) | 4 (50) | 10 (45) | 16 (48) |
| Fatal AE, excluding disease progression^{a} | 0 (0) | 1 (33) | 0 (0) | 1 (13) | 0 (0) | 1 (3) |
| Any TE CRS | 3 (100) | 2 (67) | 2 (40) | 4 (50) | 20 (91) | 27 (82) |
| Any TE CRS Grade ≥3 | 0 (0) | 0 (0) | 0 (0) | 0 (0) | 1 (5) | 1 (3) |
| Any TE neurologic event | 0 (0) | 0 (0) | 2 (40) | 2 (25) | 10 (45) | 12 (36) |
| Any TE neurologic event Grade ≥3 | 0 (0) | 0 (0) | 1 (20) | 1 (13) | 2 (9) | 3 (9) |
| Any TE cytopenia | 2 (67) | 3 (100) | 3 (60) | 6 (75) | 14 (64) | 22 (67) |
| Any TE cytopenia Grade ≥3 | 1 (33) | 2 (67) | 2 (40) | 4 (50) | 12 (55) | 17 (52) |
| Any TE infection | 2 (67) | 2 (67) | 1 (20) | 3 (38) | 5 (23) | 10 (30) |
| Any TE infection Grade ≥3 | 1 (33) | 2 (67) | 0 (0) | 2 (25) | 1 (5) | 4 (12) |
| Any TE hypogammaglobulinemia | 0 (0) | 0 (0) | 1 (20) | 1 (13) | 4 (18) | 5 (15) |
| Any TE hypogammaglobulinemia Grade ≥3 | 0 (0) | 0 (0) | 0 (0) | 0 (0) | 0 (0) | 0 (0) |
| Any TE graft-versus-host disease | 0 (0) | 0 (0) | 0 (0) | 0 (0) | 0 (0) | 0 (0) |
| Any TE graft-versus-host disease Grade ≥3 | 0 (0) | 0 (0) | 0 (0) | 0 (0) | 0 (0) | 0 (0) |
| Any TE autoimmune disorder | 0 (0) | 0 (0) | 1 (20) | 1 (13) | 3 (14) | 4 (12) |
| Any TE autoimmune disorder Grade ≥3 | 0 (0) | 0 (0) | 0 (0) | 0 (0) | 0 (0) | 0 (0) |
| Any TE tumor lysis syndrome | 0 (0) | 0 (0) | 0 (0) | 0 (0) | 0 (0) | 0 (0) |
| Any TE tumor lysis syndrome Grade ≥3 | 0 (0) | 0 (0) | 0 (0) | 0 (0) | 0 (0) | 0 (0) |

Abbreviations: AE, adverse event; CRS, cytokine release syndrome; CTCAE, Common Terminology Criteria for Adverse Events; TE, treatment-emergent; TEAE, treatment-emergent adverse event.
Cohort 1, 0.5 × 10⁶ (cells/kg); Cohort 2, 1.0 × 10⁶ (cells/kg); Cohort 3, 2.0 × 10⁶ (cells/kg).
Notes:
TEAEs include all AEs with an onset on or after the KITE-001 infusion date. Participants were summarized at their highest CTCAE grade except for CRS syndromes. Percentages were calculated using the total number of participants treated in each cohort as the denominator.
The AE preferred terms of “B-cell lymphoma”, “diffuse large B-cell lymphoma”, and “primary mediastinal large B-cell lymphoma” are considered disease progression.

### Example 8. Autoimmune Clinical Protocol

This example describes a Phase 1 open-label, multiregional, multicenter, basket study evaluating the safety and efficacy of KITE-001 in participants with SLE with or without LN, SSc, or IIM. The study will have a dose-escalation phase (Phase 1a) and a dose-expansion phase (Phase 1b).

### Phase 1a: Dose Escalation

The dose-escalation portion of the study will use a 3+3 study design. Up to 12 participants with treatment-refractory SLE with or without LN, SSc, or IIM will be enrolled and treated with KITE-001 at the sequential dose-escalation levels (shown in Table 5) and evaluated for DLTs.

There will be 2 planned dose-escalation levels (Table 5). Administration of KITE-001 in participants enrolled in Phase 1a will be staggered, as shown below, to allow for observations of acute and subacute toxicities:
Dose-escalation Level 1 (DL1): No additional participants can be dosed at DL1 until 28 days have passed since the first participant has been dosed.
Dose-escalation Level 2 (DL2): Dosing of the first participant at DL2 will be at least 28 days after the last participant has been dosed at DL1.

For participants weighing >100 kg in each dose-escalation level cohort, the maximum dose of KITE-001 CAR⁺ T cells will be capped at the dose administered for a participant body weight of 100 kg.

TABLE 5
Dose-escalation Levels for KITE-001

| | Number of | Maximum Total Dose of |
|---|---|---|
| Dose-escalation | KITE-001 CAR⁺ | CAR⁺ T Cells for |
| Level | T Cells/kg | Participants >100 kg |
| −1 | 0.2 × 10⁶ | 2 × 10⁷ |
| 1 | 0.5 × 10⁶ | 5 × 10⁷ |
| 2 | 1.0 × 10⁶ | 1 × 10⁸ |

Abbreviations: DL, dose level; MTD, maximum tolerated dose.
Notes:
Depending on the activity and tolerability observed at DL2, higher doses may also be potentially explored.

### Phase 1b: Dose Expansion

After the recommended Phase 1b dose is determined in Phase 1a, the study will progress to the Phase 1b dose expansion phase with individual indication-specific cohorts to further characterize the efficacy and safety profile. Approximately 10 participants will be treated at the target dose in each of the disease cohorts listed below. In total, up to approximately 52 participants will be enrolled and treated during the Phase 1a and Phase 1b portions of the study.
Cohort A: Lupus Nephritis
Cohort B: Systemic Lupus Erythematosus
Cohort C: Systemic Sclerosis
Cohort D: Idiopathic Inflammatory Myopathy

### Eligibility Criteria

### Inclusion Criteria

To be enrolled in the study, participants must meet all of the following criteria.

With the exception of safety laboratory values related to renal function, allowance will be made for laboratory values within 5% of cutoffs (e.g., 43% total lung capacity [TLC] and 950 cells/L absolute neutrophil count [ANC] are allowed).

### Inclusion Criteria for SLE and LN

1) Age ≥18 and ≤75 years
2) Meet the EULAR-ACR 2019 classification criteria for SLE
3) Presence of either double-stranded DNA (anti-dsDNA) and/or anti-Smith antibodies at screening per local laboratory
4) Moderate to severe, active disease defined as at least one BILAG-A score or 2 BILAG-B (excluding constitutional and/or neuropsychiatric organ system)
5) Refractory to steroids and inadequate response or intolerance to at least 2 of the following treatments, used for at least 3 months each: cyclophosphamide, mycophenolate mofetil or its derivatives, belimumab, anifrolumab, rituximab, obinutuzumab, methotrexate, azathioprine, cyclosporin, tacrolimus, or voclosporin.
   For LN: Refractory to steroids and inadequate response or intolerance to at least 2 of the following treatments, used for at least 3 months each: cyclophosphamide, mycophenolate mofetil or its derivatives, belimumab, rituximab, obinutuzumab, azathioprine, cyclosporin, tacrolimus, or voclosporin
6) Refractory disease as defined as lack of response, insufficient response or lack of sustained response to appropriate dose and treatment duration per local treatment guidelines
   For LN: refractory disease is failure to improve within 3-4 months, not achieving partial response after 6-12 months, or not achieving complete response after 2 years of treatment

### Inclusion Criteria for LN

1) Renal biopsy-proven Class III or IV±V LN according to the revised International Society of Nephrology and Renal Pathology Society (ISN/RPS) criteria within 6 months prior to or during screening
   >50% glomeruli with global sclerosis and/or >50% interstitial fibrosis and/or modified National Institute of Health (mNIH) chronicity index ≥5 on biopsy will be exclusionary
2) Evidence of active LN at screening defined as urine protein creatinine ratio (UPCR)≥0.5 based upon 24-hour urine collection per central lab evaluation
3) Progression despite maintenance on maximally tolerated doses of antiproteinuric agents per Kidney Disease: Improving Global Outcomes (KDIGO) guidelines

### Inclusion Criteria for SSc

1) Age ≥18 and ≤60 years
2) Systemic sclerosis according to ACR/EULAR 2013 classification criteria
3) Disease duration ≤5 years from the first non-Raynaud's phenomenon manifestation
4) Modified Rodnan Skin Score (mRSS)>10 with early disease <18 months
5) mRSS≥15 with disease duration ≥18 months OR
6) Increase ≥3 in mRSS score over 6 months or less
7) Either a Health Assessment Questionnaire-Disability Index (HAQ-DI) score ≥0.25 points or patient global assessment score ≥3 points
8) High-resolution computer tomography (HRCT) scan and pulmonary function test (PFT) within 3 months prior to screening to evaluate SSc-interstitial lung disease (ILD):
   A) TLC>45%
   B) For ILD: forced vital capacity (FVC) >45%, diffusing capacity of the lungs for carbon monoxide (DLCO) (corrected for hemoglobin) >40% predicted, and/or baseline resting systolic pulmonary artery pressure (PAP)<40 or mean pulmonary arterial pressure (mPAP)<25 mmHg by echocardiography
9) Refractory or intolerance to 1 of the following for a minimum of 3 months and/or contraindication: mycophenolate mofetil or its derivatives, methotrexate, tocilizumab (or other IL-6 inhibitor), rituximab (or other B-cell depleting agent), nintedanib (or other antifibrotic agents), cyclophosphamide

### Inclusion Criteria for IIM

1) Age ≥18 years
2) Idiopathic inflammatory myopathy (IIM) based on EULAR/ACR 2017 classification (excluding inclusion body myositis)
3) Active disease by electromyography (EMG) or magnetic resonance imaging (MRI) within 3 months of lymphodepletion
4) Moderate to severe disease activity demonstrated by manual muscle testing in 8 muscles (MMT-8)≤142 AND at least 2 of the following core set measures abnormalities:
   A) Patient/Parent Global Activity Assessment ≥2 cm
   B) Physician Global Activity Assessment using Myositis Disease Activity Assessment Tool (MDAAT) ≥2 cm
   C) Extramuscular Activity Assessment derived from MDAAT ≥2 cm
   D) HAQ-DI ≥0.25
   E) Abnormal muscle enzymes (>upper limit of normal [ULN]) demonstrated by at least 1 of the following: creatine kinase (CK), aldolase, lactate dehydrogenase, aspartate aminotransaminase (AST), alanine aminotransferase (ALT)
5) Positive for myositis specific antibodies for patients with non-dermatomyostitis IIM
6) HRCT scan and PFT within 3 months prior to screening to evaluate for presence of IIM-ILD
   A) TLC>45%
   B) For ILD: FVC >45%, DLCO (corrected for hemoglobin) >40% predicted, and/or baseline resting systolic PAP <40 or mPAP <25 mmHg by echocardiography
7) Negative malignancy screening per local guidelines within the last 2 years.
8) Active disease on standard of care (SOC), must meet 1 of the criteria prior to screening:
   Refractory or intolerance to at least 1 month of glucocorticoids and standardized use of at least 2 immunosuppressant/modulator (e.g., intravenous gamma globulins, methotrexate, mycophenolate mofetil and its derivatives, azathioprine, cyclophosphamide, calcineurin inhibitors, JAK inhibitors, rituximab or other B-cell depleting agent)

### Inclusion Criteria for all Cohorts

1) Meet the washout periods for relevant medications before leukapheresis
2) Participants must agree to use protocol-specified method(s) of contraception where applicable. Participants who have undergone surgical sterilization or have been postmenopausal for at least 2 years before enrollment are not considered to be of childbearing potential.

### Exclusion Criteria (All Cohorts)

To be enrolled in the study, participants must not meet any of the following criteria:
1) Females of childbearing potential who are pregnant or breastfeeding
2) Unstable cardiac disease within 12 months before enrollment:
   A) Cardiac ejection fraction <50% via echocardiogram (ECHO) or multigated acquisition scan (MUGA)
   B) Hemodynamic instability secondary to large pericardial effusion or cardiac tamponade
   C) Unstable arrythmia, valvular dysfunction, myocarditis, coronary vasculitis, or clinically significant electrocardiogram (ECG) findings, and/or
   D) Myocardial infarction, cardiac angioplasty or stenting, unstable angina
3) Clinically significant pulmonary disease within 12 months before enrollment:
   A) Baseline oxygen saturation <92% on room air
   B) Pulmonary hemorrhage
4) eGFR <40 mL/min/1.73 m²using the Chronic Kidney Disease (CKD) Epidemiology Collaboration (CKD-EPI) formula at screening
5) Dialysis within the past year
6) History of symptomatic deep vein thrombosis or pulmonary embolism requiring therapeutic anticoagulation within 6 months before enrollment
7) History of malignancy, including autoimmune lymphoproliferative disorders, within the last 5 years, except for adequately treated cancers of the skin (basal or squamous cell) or carcinoma in situ of the cervix
8) Bone marrow insufficiency unrelated to active SLE, SSc, or IIM within 28 days of lymphodepletion:
   A) Absolute neutrophil count ≤1000/μL
   B) Platelet count ≤75,000/μL
   C) Absolute lymphocyte count ≤500/μL
   D) Hemoglobin ≤8/μL
9) AST, ALT >2 ULN (unless secondary to muscle related elevation related to diagnostically proven IIM and after ruling out cirrhosis or liver disease not related to IIM)
10) Bilirubin >1.5 mg/dL (except in participants with Gilbert's syndrome where ≤3.0 ULN is acceptable)
11) Hypogammaglobulinemia requiring immunoglobulin replacement per local guidelines (IVIG treatment for IIM allowed, per washout criteria)
12) Active smoking
13) Active alcoholism
14) History of autologous or allogeneic stem cell transplant and/or organ transplant
15) Prior treatment with cellular therapy, gene therapy and/or T-cell engager therapy
16) Treatment with any investigational agent within 28 days or 5 half-lives, whichever is longer, prior to leukapheresis
17) History of severe, immediate hypersensitivity reaction attributed to aminoglycosides or history of severe hypersensitivity reaction to any of the agents used in this study
18) Known history of HIV infection, hepatitis B virus (HBV) (hepatitis B surface [HBs] antigen [HBsAg] positive) infection, or hepatitis C (hepatitis C ribonucleic acid [RNA] positive) infection. History of a hepatitis B or C infection is permitted if the viral load is undetectable per quantitative polymerase chain reaction (PCR) or nucleic acid testing. Note: Subjects who are seropositive for HBV (ie, HBs and/or hepatitis B core antibody positive) are eligible if they are HBsAg-negative and negative for viral DNA. Subjects who are seropositive because of HBV vaccination are eligible (ie, HBs antibody positive, hepatitis core antibody-negative, and HBsAg-negative). Subjects on prophylactic and suppressive antiviral medications against HBV and/or HCV administered per institutional or clinical practice guidelines are eligible.
19) Active or untreated latent tuberculosis (TB)
20) Live vaccine ≤6 weeks before planned start of lymphodepleting chemotherapy regimen or participants without up-to-date immunization per local guidelines
21) Presence of fungal, bacterial, viral, or other infection that is uncontrolled or requires IV antimicrobials for management. Note: Simple urinary tract infections and uncomplicated bacterial pharyngitis are permitted if the participant is responding to active treatment and after consultation with the Kite medical monitor.
22) Known major surgery requiring hospitalization within 4 weeks of the planned treatment start date
23) Any medical condition likely to interfere with the assessment of safety or efficacy of study treatment
24) In the investigator's judgment, the participant is unlikely to complete all protocol-required study visits or procedures, including follow-up visits, or comply with the study requirements for participation
25) Nonspecific, overlap, mixed autoimmune diseases not clearly identified into any of the studied cohorts
26) Symptomatic pleural effusion requiring intervention (e.g., diuretics or therapeutic thoracentesis)

### Exclusion Criteria for LN

1) Presence of crescent formation in ≥50% of glomeruli assessed on renal biopsy or >50% glomeruli with global sclerosis and/or >50% interstitial fibrosis and/or NIH chronicity index ≥5 on biopsy, and/or sustained doubling of serum creatinine within 12 weeks of screening or based on investigator's opinion that the participant has rapidly progressive glomerulonephritis
2) Pure class VI nephritis
3) Participants with severe proteinuria associated with clinically significant hypoalbuminemia and/or severe fluid imbalance (such as edema, anasarca, or ascites) as determined by the investigator's clinical judgment

### Exclusion Criteria for SLE

1) Drug-induced SLE
2) Catastrophic antiphospholipid syndrome
3) Participants with antiphospholipid syndrome and a history of arterial thrombosis or recent thrombosis (≤6 mo)
4) Thrombotic thrombocytopenic purpura
5) Active or unstable lupus neuropsychiatric manifestations including, but not limited to, any conditions defined by BILAG within last 6 months

### Exclusion Criteria for SSc

1) Severe Raynaud's with digital gangrene or active infection of digital ulcer within 3 months prior to enrollment
2) Severe pulmonary hypertension and/or requiring an intravenous prostaglandin agonist
3) History of systemic sclerosis renal crisis within 12 months prior to enrollment
4) History of active bleeding related to gastric antral vascular ectasia within 6 months prior to enrollment

### Exclusion Criteria for IIM

1) Other inflammatory and noninflammatory myopathies: inclusion body myositis, amyopathic dermatomyositis (DM), overlap myositis, metabolic myopathies, muscle dystrophies, or a family history of muscle dystrophy, drug induced, papular mucinosis (PM) associated with HIV or endocrine induced myositis
2) Severe, irreversible muscle damage (related or unrelated to IIM) where the benefit-risk profile is not acceptable per the discretion of the investigator

### Example 9. Functional Characterization of KITE-001 Prepared from Autoimmune Rheumatic Diseases

Supplementary to Examples 4 and 5, this example evaluated the functions of KITE-001 prepared from healthy donors or patients with systemic lupus erythematosus (SLE) with (n=7) and without lupus nephritis (LN) (n=7), or systemic sclerosis (SSc) (n=6). The CAR preparation methods are as described above.

T-cell products were co-cultured with negative fraction (remaining apheresis sample after T cells were isolated for manufacturing) that contains autologous B cells and various cell lines, including Raji (CD19⁺CD20⁺), Raji CD19KO (CD19-CD20⁺), Raji CD20KO (CD19⁺CD20⁻), and K-562 (CD19⁻CD20⁻). Functional read-outs included cytotoxic activity (luciferin and flow cytometry) on days 1 and 4, cytokine production (MSD assay) on day 1, and T-cell proliferation (Cell Trace™ Violet dye dilution by flow cytometry) on day 4.

### B Cell Cytotoxicity

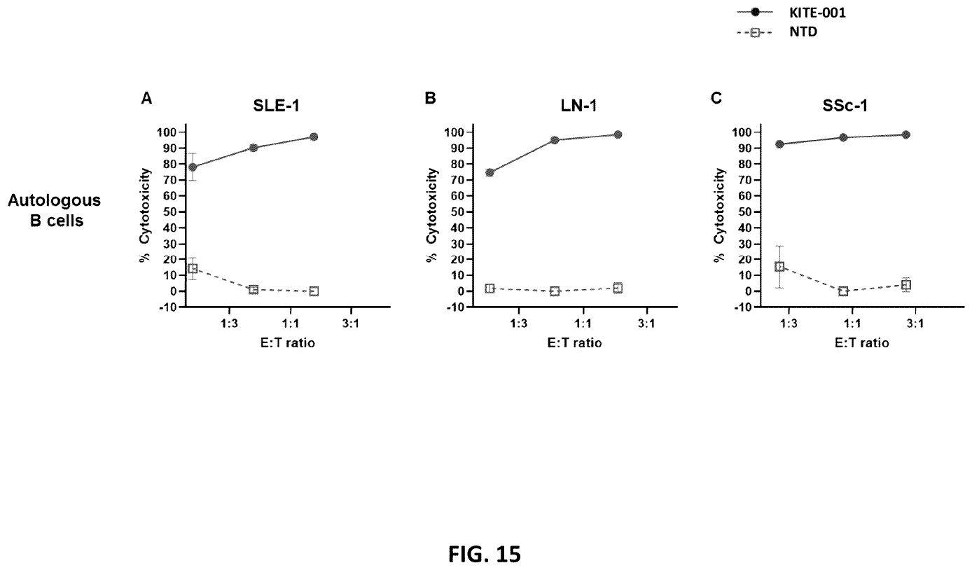

Flow cytometry-based cytotoxicity of patient-derived KITE-001 products and NTD controls against autologous B cells was evaluated. The results are shown in FIG. 15. Since target B cells constitute only a portion of the negative fraction, B-cell counts on Day 0 of co-culture were used to calculate the actual effector to target B-cell ratios. T cells were co-cultured with autologous B cells at various E:T ratios of (FIG. 15A) 1.5:1, 1:2, and 1:6 for SLE-1, (FIG. 15B) 1.8:1, 1:1.7, and 1:5 for LN-1, and (FIG. 15C) 2.5:1, 1:1.2, and 1:3.5 for SSc-1. Co-culture assays were performed using 3 technical replicates. Graphs denote mean±standard deviation in the figure.

### Cytotoxicity Against Target Cell Lines

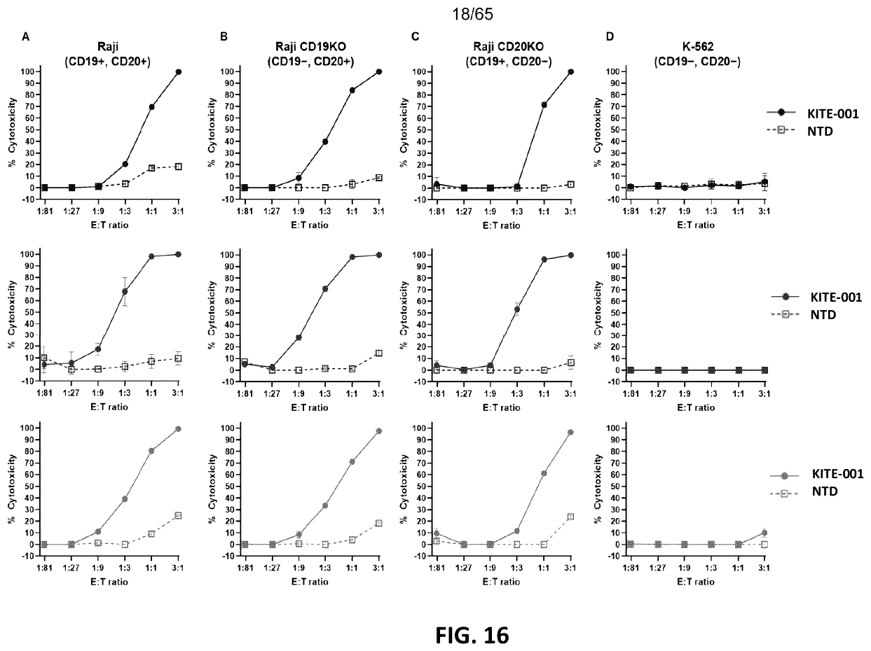

KITE-001 T-cell products and NTD controls were co-cultured with various CD19 and CD20 antigen-positive cell lines including (FIG. 16A) Raji (CD19⁺CD20⁺), (FIG. 16B) Raji CD19KO (CD19⁻CD20⁺), (FIG. 16C) Raji CD20KO (CD19⁺CD20⁻), and (FIG. 16D) K-562 (CD19⁻CD20⁻) at various effector to target (E:T) ratios. After 4 days, cytotoxicity was measured via luciferin viability read-out. As shown in FIG. 16A-D, KITE-001 exhibited antigen-specific cytotoxicity.

### Antigen-Specific Proliferation

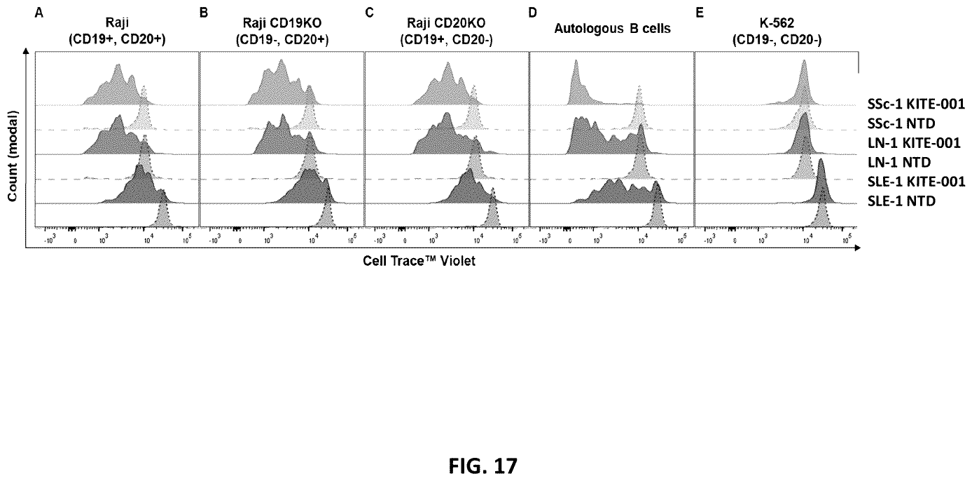

Cell Trace™ Violet-labeled (CTV) KITE-001 and NTD T cells were cocultured with (FIG. 17A) Raji (E:T 1:1), (FIG. 17B) Raji CD19KO (E:T 1:1), (FIG. 17C) Raji CD20KO (E:T 1:1), (FIG. 17D) autologous B cells (E:T 1:2, 1:1.7, and 1:1.2 for SLE-1, LN-1, and SSc-1 respectively), or (FIG. 17E) K-562 cells (E:T 1:1) for 4 days. Proliferation of the T-cell products was determined by flow cytometric assessment of CTV dilution. As shown in FIG. 17A-E, the KITE-001 products had efficient antigen-specific proliferation.

### Cytokine Production Against Antigen-Positive Cells

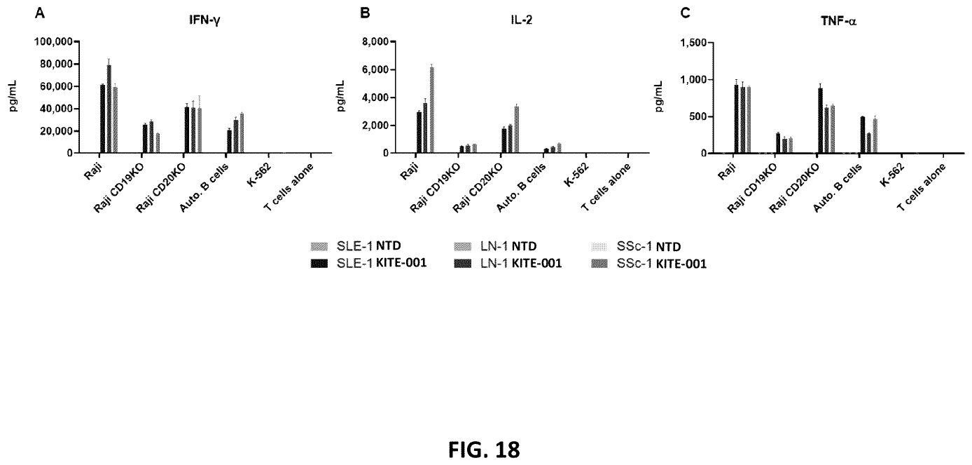

FIG. 18A-C show levels of (A) IFN-γ, (B) IL-2, and (C) TNF-α pro-inflammatory cytokines secreted by KITE-001 T-cell products at Day 1 of co-culture with Raji cells (E:T 1:1), Raji CD19KO cells (E:T 1:1), Raji CD20KO cells (E:T 1:1), autologous B cells (E:T 1:2, 1:1.7, and 1:1.2 for SLE-1, LN-1, and SSc-1 respectively), K-562 (E:T 1:1) cells, or T cells alone. These results demonstrate that KITE-001 effectively produced various cytokines against antigen-positive cells.

In this example, KITE-001 prepared from patients with various autoimmune rheumatic diseases, including systemic lupus erythematosus (SLE) with and without lupus nephritis (LN), or systemic sclerosis (SSc), demonstrated CAR- and antigen-specific T-cell functionality: the CAR-T cell products exhibited cytokine production, proliferation, and dose-dependent cytotoxicity against autologous primary B cells and cell lines expressing either CD19 and/or CD20 antigens.

### Example 10. Functional Characterization of KITE-001 Manufactured from Patients with Myasthenia Gravis

This example assessed manufacturability of KITE-001 from T cells isolated from apheresis material of two patients with myasthenia gravis (MG-013 and MG-579).

### Methods

### Lentiviral Titering

Anti-CD19/CD20 LVV was produced and titered using standard, in-house methods. Briefly, Jurkat cells were transduced with serially diluted LVV. On Day 3 post-transduction, Jurkat cells were stained with an antibody that binds the Whitlow linker between the light and heavy chains of the single chain variable fragment (scFv) portion of both CARs and analyzed by flow cytometry. The viral dilution that yields 5-30% CAR-positivity is used to calculate viral titers (number of infectious particles per mL).

### T-Cell Activation, Transduction, and Expansion

Isolated CD4⁺ and CD8⁺ primary human T cells were cultured in OpTmizer CTS T-cell expansion basal media supplemented with OpTmizer CTS T-cell expansion supplement, CTS immune cell serum replacement, penicillin/streptomycin/L-glutamine, and human interleukin (IL)-2. T cells were resuspended in the OpTmizer media containing anti-CD28 antibody and then seeded into a cell culture bag precoated with an anti-CD3 antibody to induce T-cell activation (Day 0). On Day 1 post-activation, cells were either transduced with an LVV encoding a bicistronic construct with coding regions to generate KITE-001 CAR T-cell products or were left NTD to serve as a negative control. T cells were washed with the OpTmizer media on Day 3 and cells were expanded for an additional 6 days (Days 3 to 9). At the harvest time point (Day 9), cells were cryopreserved for co-culture experiments to assess CAR T-cell function. At multiple time points during expansion (Days 3 to 9), cell counts were taken using a Vi-CELL BLU and the cell density was normalized down to 0.5×106 cells/mL by addition of the OpTmizer media. At multiple time points (Days 0 to 9), average cell viability, average diameter, and average cell density were recorded on the Vi-CELL BLU.

### Flow Cytometry to Determine CAR Transduction Efficiency and T-Cell Phenotype

CAR transduction efficiency and T-cell phenotype were evaluated using samples of T-cell products at various time points throughout the manufacturing process. T cells were stained with a panel of fluorophore-conjugated antibodies and characterized by flow cytometry to determine transduction efficiency, CD4⁺ and CD8⁺ T cell ratios, and memory subset ratios based on the expression of CD45RA and CCR7. Expression of anti-CD19 and anti-CD20 CARs was measured by flow cytometry. A fixable cell viability dye allowed specific analysis of viable cells. Cells were blocked at room temperature for 10 minutes. Cells were then subsequently stained by incubating with the appropriate antibody mix for 30 minutes at 4° C. followed by 2 washes with stain buffer, and subsequently fixed at room temperature. Fixed samples were analyzed by flow cytometry on each sampling day.

### Target Cell Lines

Raji (Burkitt's B-cell lymphoma) cells expressing both CD19 and CD20 proteins on the cell surface were used as target cells to assess the functional activity of KITE-001 T-cell products. The Raji parental cell line was engineered to remove MHC I/II expression via targeted double knock out to reduce the potential for alloreactivity. Furthermore, the Raji cell line was engineered through a second knock out to remove expression of CD19 or CD20 (CD19KO and CD20KO, respectively). K-562 (chronic myelogenous leukemia) cells lacking expression of MHC I/II, CD19, and CD20 were used as an antigen negative control line. Cell lines were additionally engineered (lentiviral transduction under antibiotic selection) to express luciferase as a tool for viability read out.

### Co-Culture Setup

Cryopreserved T-cell products were thawed and rested overnight in RPMI-1640 media prior to co-culture assays. After overnight rest, KITE-001 and NTD T cells were washed, labeled with Cell Trace™ Violet (CTV) dye, and co-cultured with luciferase-expressing target cells or patient-derived negative fraction cells containing autologous primary B cells. The negative fraction consists of the remaining white blood cells (from leukopaks) after T cells had been isolated to generate the CAR T-cell products.

Prior to co-culture setup, a sample of each T-cell product was incubated with a panel of antibody-fluorophore conjugates and analyzed by flow cytometry to assess total CAR expression by using KIP-1, a custom-made antibody that binds to the Whitlow linker between the heavy and light chains of the single-chain variable fragment (scFv) of both the anti-CD19 CAR and the anti-CD20 CAR. Total CAR expression of KITE-001 products were made comparable by addition of patient-specific NTD T cells. T-cell products were then labeled with Cell Trace™ Violet (CTV) dye and washed with R-10% media. CTV-labeled T-cell products were plated with luciferase-expressing target cells at different effector to target (E:T) ratios ranging from 3:1 to 1:81 in media (Day 0 of co-culture), and autologous negative fraction cells at different E:T ratios ranging from 3:5 to 1:15. T-cell products were serially diluted 3-fold while the number of luciferase-expressing target cells per well was held constant at 25,000 cells whereas negative fraction cells were held constant at 125,000 cells. Antigen-expressing cell lines included Raji (CD19⁺CD20⁺), Raji CD19KO (CD19⁻ CD20⁺), and Raji CD20KO (CD19⁺CD20⁻). K-562 (CD19⁻ CD20⁻) was used as a negative control cell line. NTD control T cells were used to establish the background level of homeostatic responses due to non-specific target cell killing. As an additional control, T-cell products were cultured without target cells (i.e., T cells alone) to assess basal levels of T cell function without stimulation. Co-cultures were incubated at 37° C. for either 1 or 4 days, and functional assessments were performed.

### Cytotoxicity

T cell-mediated cytotoxicity was measured as the reduction in target-specific luciferase signal in co-culture wells compared with target cells plated alone. On Days 1 and 4 of co-culture, D-luciferin substrate was added to wells at a final concentration of 0.14 mg/mL followed by an incubation at 37° C. in the dark for 10 minutes. Luminescent signal was read immediately after using an Envision multimode microplate reader.

For unlabeled negative fraction cells containing autologous primary B cells, T cell-mediated cytotoxicity was measured as the reduction of absolute B cell counts in co-culture wells compared with target cells plated alone using flow cytometry. B cell counts in the negative fraction were assessed prior to coculture setup (Day 0), and after coculture initiation (Days 1 and 4) by using an antibody against the CD22 antigen. On Days 0, 1 and 4, cells were harvested and incubated with antibody fluorophore conjugates.

### Cytokine Production

On Day 1 after co-culture initiation, supernatants were collected and analyzed for cytokine levels using V-Plex Proinflammatory Panel 1 Human kit according to the manufacturer's (Meso Scale Discovery) instructions. Specifically, supernatants from 1:1 E:T co-culture ratios with target cell lines, and autologous B cells at 1:1.2 for MG-579 were analyzed for levels of interferon gamma (IFN-γ), interleukin 2 (IL-2), and tumor necrosis factor alpha (TNF-α) secretion mediated by antigen engagement. Supernatants from T cells co-cultured with antigen-negative K-562 target cells were analyzed in parallel with T cells alone (NTD and KITE-001) to assess antigen-independent cytokine production levels. All samples were diluted to appropriate levels within the range of assay detection.

### Proliferation

On Day 4 after co-culture initiation, the proliferative capacity of T-cell products exposed to patient-derived negative fraction cells containing autologous primary B cells and target cell lines was determined by flow cytometric analysis of cell division (via dilution of Cell Trace Violet™ dye) and compared with NTD control T cells. T cells co-cultured with antigen-negative K-562 (CD19⁻ CD20⁻) cells were used to assess specificity and levels of antigen-independent proliferation while T cells alone were used to assess basal levels of proliferation in the absence of stimuli. Specifically, T-cell products plated at 1:1 E:T ratio with target cell lines, at 1:1.2 with autologous B cells for MG-579 (were harvested and incubated with fluorophore-conjugated antibodies to identify Cell Trace Violet™ (CTV)-labeled T cells by flow cytometry.

### Results

### T-Cell Product Viability

The viability of the T-cell products was assessed from Days 0 to 9. Cell viability was greater than 70% for all MG-013 T-cell products (FIG. 19A) and greater than 62% for all MG-579 T-cell products (FIG. 19B) between Days 0 and 9.

### T-Cell Product Activation (Cell Diameter)

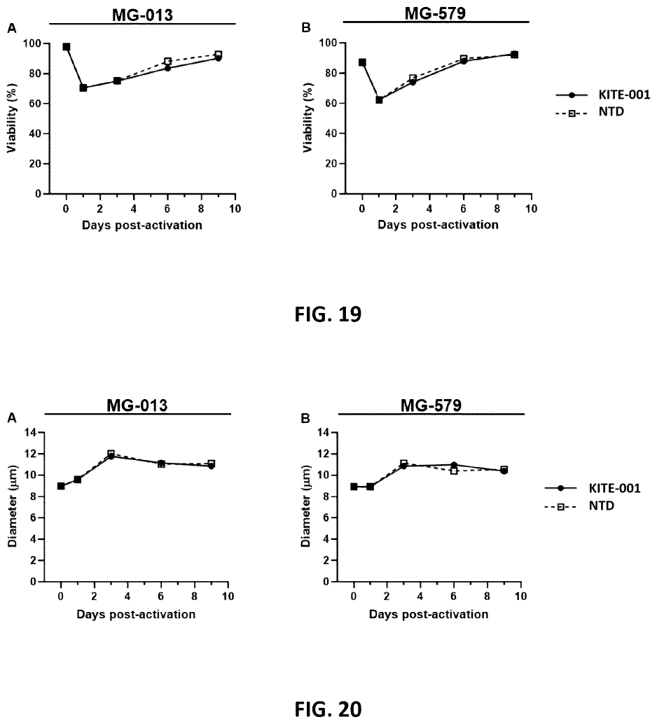

Cell diameter was measured for all T-cell products at multiple time points between Days 0 and 9 as an indicator of T-cell activation. Cell blasting was observed upon activation for all T-cell products, as evidenced by an increase in cell diameter between Days 1 and 3. The diameters of each patient's T-cell products were comparable on the days they were measured during the manufacturing process (FIG. 20A-B).

### T-cell Product Expansion

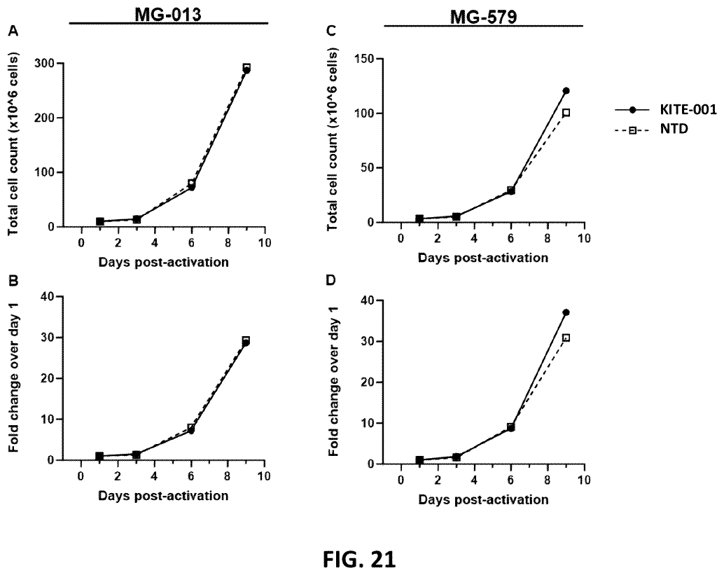

During manufacture of KITE-001 products, T cells were counted and the average total cell count (FIGS. 21A and C) and fold change in cell count compared with Day 1 (day of transduction; FIGS. 21B and D) were calculated. From Days 1 to 9, MG-013 KITE-001 product expanded 29-fold, and the corresponding NTD product also expanded 29-fold. MG-579 KITE-001 product expanded 37-fold and the corresponding NTD product expanded 31-fold.

### Transduction Efficiency of T-Cell Products

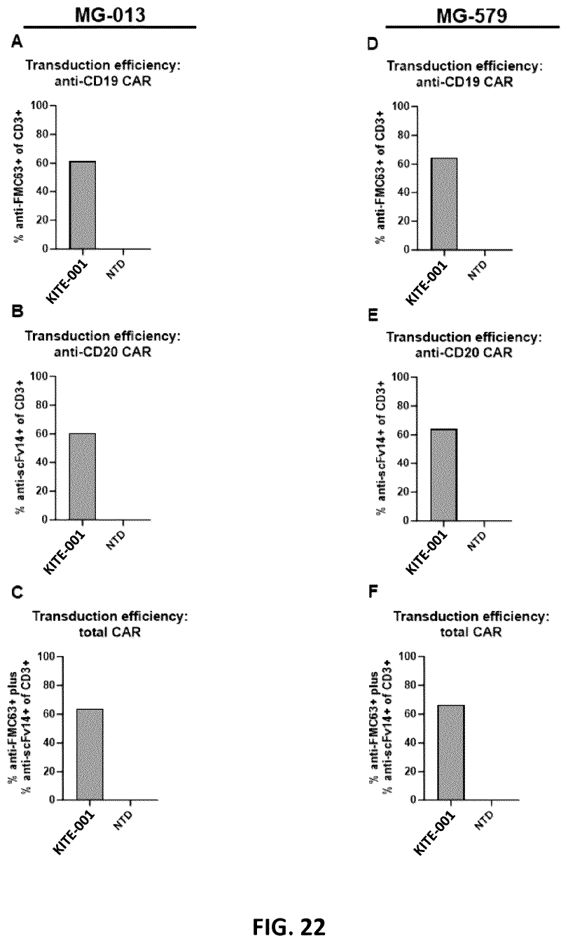

CAR transduction efficiency was evaluated using samples of T-cell products on Day 9 (harvest day). Individual expression of the anti-CD19 CAR in the KITE-001 products based on anti-FMC63 staining was 60% for MG-013 and 65% for MG-579 (FIGS. 22A and D), and 58% and 64% respectively, for the anti-CD20 CAR detected by anti-scFv14 staining in the same samples (FIGS. 22B and E). Total CAR expression of KITE-001 products, as measured by the sum of total anti-CD19 and anti-CD20 CAR expression (calculated by the sum of cells that are % anti-FMC63+ and/or % anti-scFv14+), was 62% and 67% for MG-013 and MG-579, respectively (FIGS. 22C and F). In contrast, NTD control T cell samples did not stain positively for either anti-CD19 or anti-CD20 CAR expression.

### Phenotype of T-Cell Products

In addition to CAR transduction efficiency, the proportions of CD4⁺ and CD8⁺ T cells were tracked between Days 0 and 9 (FIG. 23). In all T-cell products for MG-013 and MG-579 and as a result of the manufacturing process used for the generation of the T-cell products, an enrichment in the CD8⁺ T-cell population was observed (compared to Day 0).

The proportions of T-cell memory and effector subsets (naive, central memory, effector memory, and terminally differentiated effector memory), as determined by differential CD45RA and CCR7 expression, were assessed in T-cell products between Days 0 and 9 (FIG. 24). Compared with Day 0, an enrichment in the effector memory subset was observed for all T-cell products for MG-013 and MG-579 by Day 9.

KITE-001 CAR T-cell products transduced with a bicistronic LVV encoding an anti-CD19 CAR and an anti-CD20 CAR were successfully generated from CD4⁺ and CD8⁺ T cells that had been positively selected from apheresis material from two patients with MG. The KITE-001 T-cell products increased in diameter upon activation, expanded, and maintained high viability throughout the manufacturing process. Flow cytometry-based analysis on Day 9 (harvest day) demonstrated that the T cells expressed both the anti-CD19 CAR and anti-CD20 CAR and that total transduction efficiency was 62% and 67% for MG-013 and MG-579 KITE-001 products, respectively.

### CD19/CD20 Expression in Patients B Cells

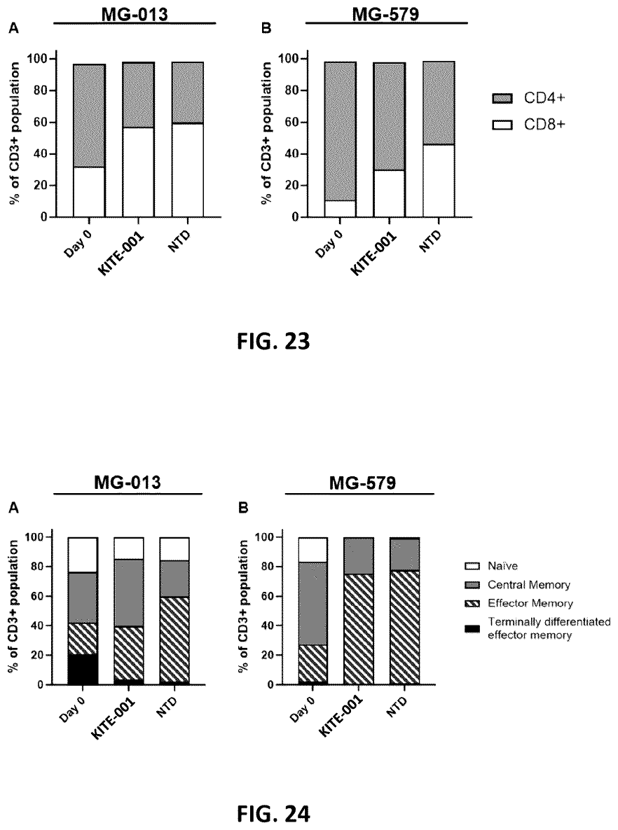

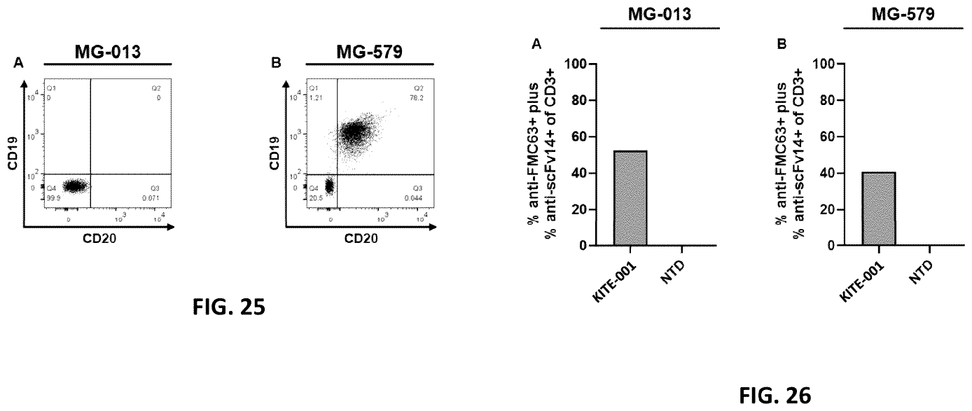

Antigen expression on autologous B cells from patients with MG was evaluated by flow cytometry using commercially available fluorophore-conjugated antibodies. CD19⁺CD20⁺ B cells were detected in MG-579 (FIG. 25B), however no CD19⁺CD20⁺ B cells were detected in MG-013 likely due to the patient's rituximab treatment (FIG. 25A).

### Total CAR Expression

Total CAR expression for both KITE-001 T-cell products was reduced by addition of patient-specific NTD T cells. Immediately prior to co-culture setup, total CAR expression was assessed at 53% for MG-013 and 41% for MG-579 (FIGS. 26A and B).

### Cytotoxicity

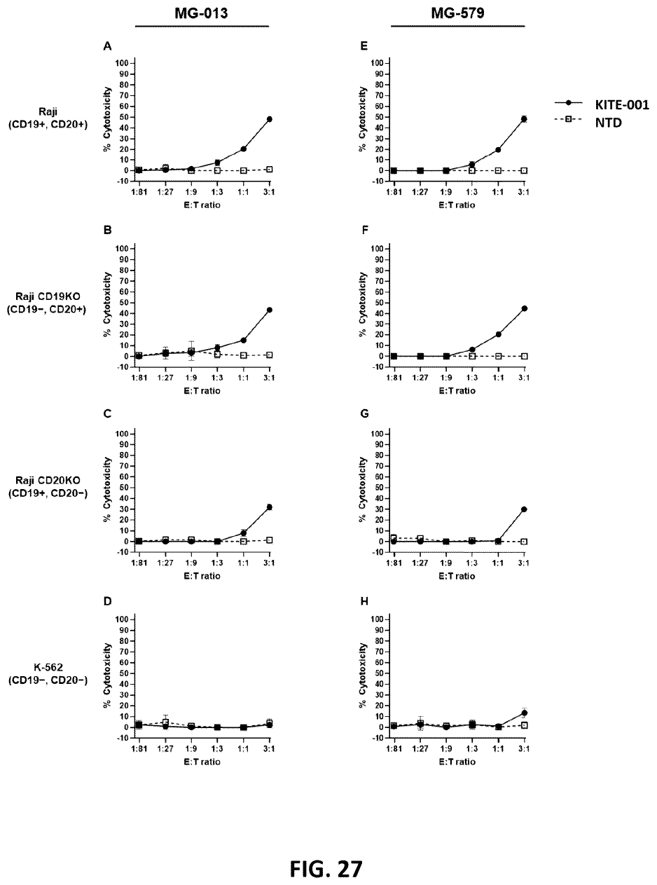

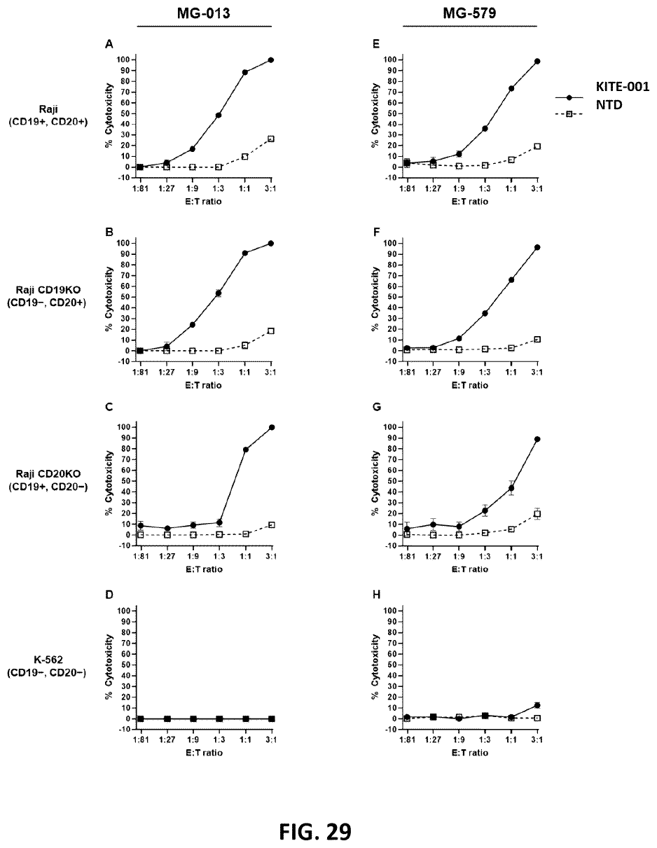

Through a luciferase-based killing assay, KITE-001 products generated from patients with MG demonstrated dose (E:T Ratio)-dependent cytotoxic activity against dual and single antigen-positive Raji target cells beginning at Day 1 (FIGS. 27A-C and E-G) and reaching maximum cytotoxic activity at the highest E:T ratio (3:1) by Day 4 of co-culture (FIGS. 27A-C and E-G). In contrast, NTD control T cells were nonreactive against all the antigen-expressing target cells on Day 1 and demonstrated minimal levels of antigen-independent activity against Raji target cells at higher E:T ratios on Day 4 (FIG. 27 and FIG. 29). Co-culture with K-562 cells showed lack of cytotoxicity in the absence of both antigens at Day 1 and Day 4 (FIG. 27 and FIGS. 29D and H).

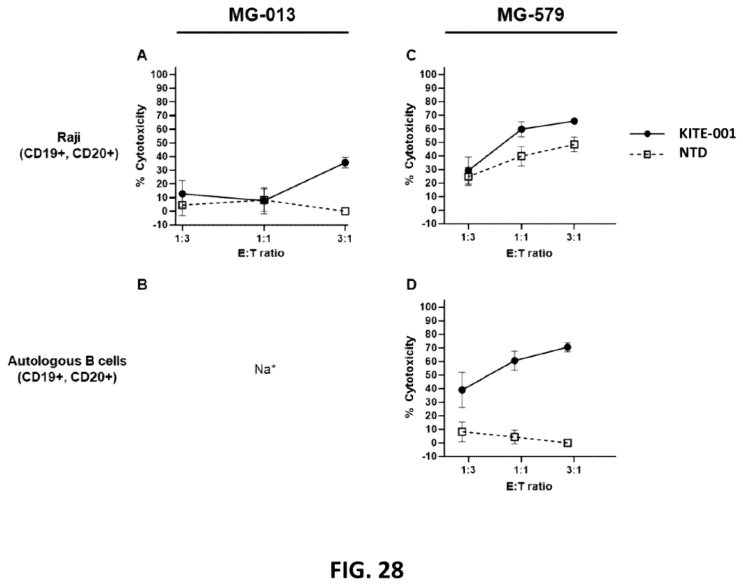

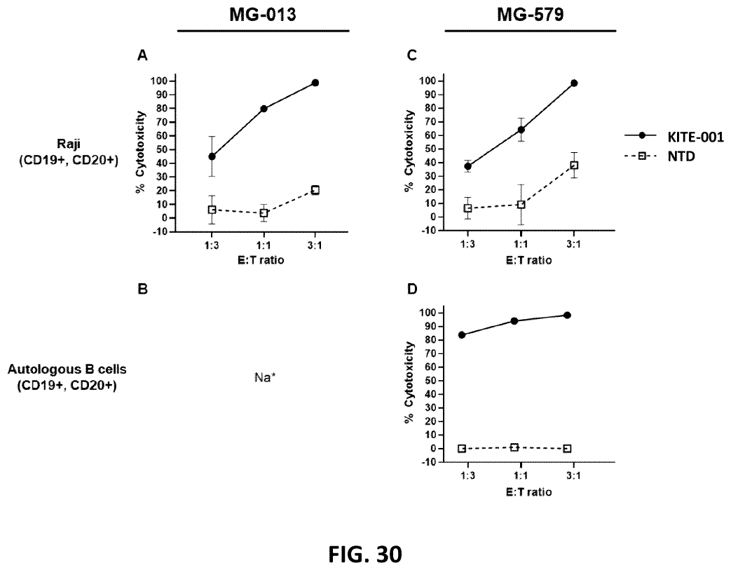

Through a flow cytometry-based killing assay, KITE-001 product from MG-579 exhibited dose (E:T ratio)-dependent specific cytotoxicity against autologous B cells beginning at Day 1 (FIG. 24), which reached maximum cytotoxic activity at the highest E:T ratio by Day 4 of coculture (FIG. 30). Minimal to no cytotoxicity was observed with NTD control T cells on Days 1 and 4 (FIG. 28 and FIG. 30). To verify robustness of detecting cytotoxicity of target cells by flow cytometry, Raji cells were used as a control to demonstrate that cytotoxicity of Raji cells from both the luciferase and flow cytometry-based killing assays were dose-dependent.

Combined, these results demonstrate that KITE-001 products from patients with MG exhibit robust antigen-dependent and antigen-specific cytotoxicity in coculture with target cells, including autologous B cells.

### Cytokine Production

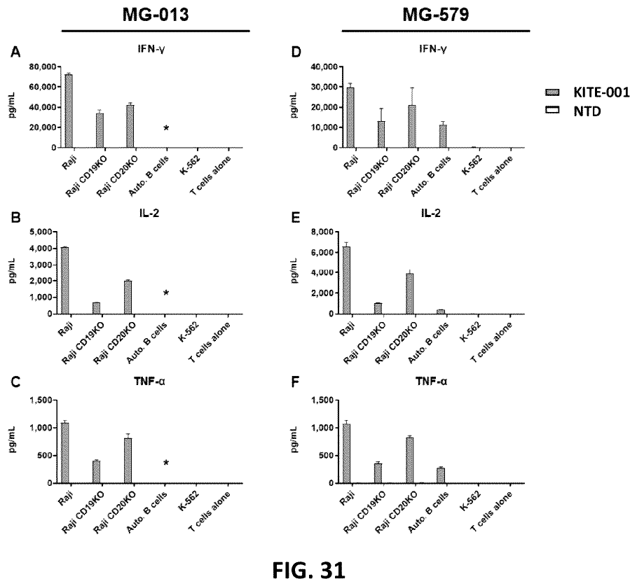

KITE-001 T-cell products yielded measurable levels of IFN-γ, IL-2, and TNF-α pro-inflammatory cytokines with stimulation by CD19 only, CD20 only, or CD19/CD20 expressing target cell lines. Importantly, KITE-001 products from MG-579 also exhibited cytokine production in response to autologous B cells. In contrast, cytokine production was minimal or absent when NTD T cells were co-cultured with any target cells (FIG. 31).

Pro-inflammatory cytokine production was specific, as no cytokine production was detected in the absence of target cells (i.e., T cells alone) and there was no cytokine production from KITE-001 T-cell products co-cultured with antigen-negative K-562 (CD19⁻CD20⁻) cells, as shown (FIG. 27). Therefore, these results demonstrate that KITE-001 products from patients with MG showed antigen-dependent- and antigen-specific production of IFN-γ, IL-2, and TNF-α in co-culture with target cells, including autologous B cells.

### Proliferation

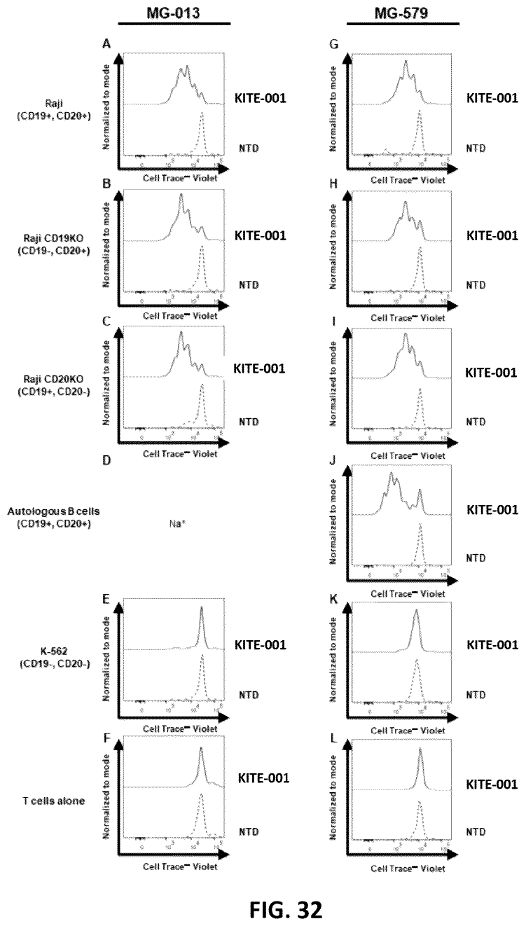

KITE-001 products from patients with MG showed robust proliferation when co-cultured for 4 days with single antigen (Raji CD19KO and Raji CD20KO) and dual antigen-expressing (Raji) target cell lines (FIGS. 32A-C and G-I), but not with antigen-negative K-562 cells (FIGS. 32E and K), indicating target-specific proliferation. Importantly, KITE-001 products from MG-579 exhibited robust proliferation in response to autologous B cells (FIG. 32J). Furthermore, NTD cells displayed minimal to no non-specific proliferation across all co-culture conditions (FIGS. 32A-C and G-J), and T cells cultured alone (FIGS. 32F and L). The CTV MFI of KITE-001 T-cell products was at least 1 log-fold lower when co-cultured with antigen-positive target cells demonstrating antigen-specific proliferation. Overall, these results demonstrate that KITE-001 products from patients with MG showed robust antigen-dependent and antigen-specific proliferation from co-culture with antigen-positive target cells, including autologous B cells.

In this example, KITE-001 products generated from patients with MG exhibited in vitro activity when challenged with CD19 and/or CD20 antigen-expressing target cells. In vitro assays demonstrated CAR and antigen-specific T-cell functionality: the CAR T-cell products exhibited cytokine production, proliferation, and dose-dependent cytotoxicity against autologous primary B cells and cell lines expressing either CD19 and/or CD20 antigens, but not against cell lines that did not express either antigen. NTD products lacking CAR expression did not react with any of the target cell lines or autologous B cells. These results support antigen-specificity and B-cell depleting activity of KITE-001 manufactured from patients with MG.

### Example 11. Functional Characterization of KITE-001 Manufactured from Patients with Multiple Sclerosis

This example assessed manufacturability of KITE-001 from T cells isolated from apheresis material of two patients with multiple sclerosis (MS-087 and MS-607). T-cell harvesting, activation, transduction, expansion and evaluation were conducted similar to Example 10.

### Results

### T-Cell Product Viability

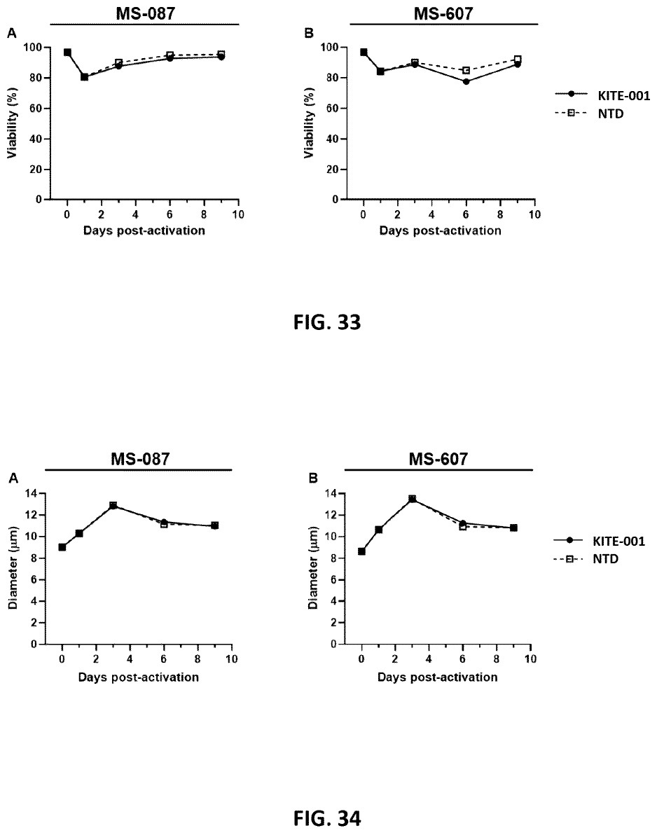

The viability of the T-cell products was assessed from Days 0 to 9. Cell viability was greater than 80% for all MS-087 T-cell products (FIG. 33A) and greater than 77% for all MS-607 T-cell products (FIG. 33B) between Days 0 and 9.

### T-Cell Product Activation (Cell Diameter)

Cell diameter was measured for all T-cell products at multiple time points between Days 0 and 9 (FIG. 34) as an indicator of T-cell activation. Cell blasting was observed upon activation for all T-cell products, as evidenced by an increase in cell diameter between Days 1 and 3. The diameter of each patient's T-cell products were comparable on the days they were measured during the manufacturing process.

### T-Cell Product Expansion

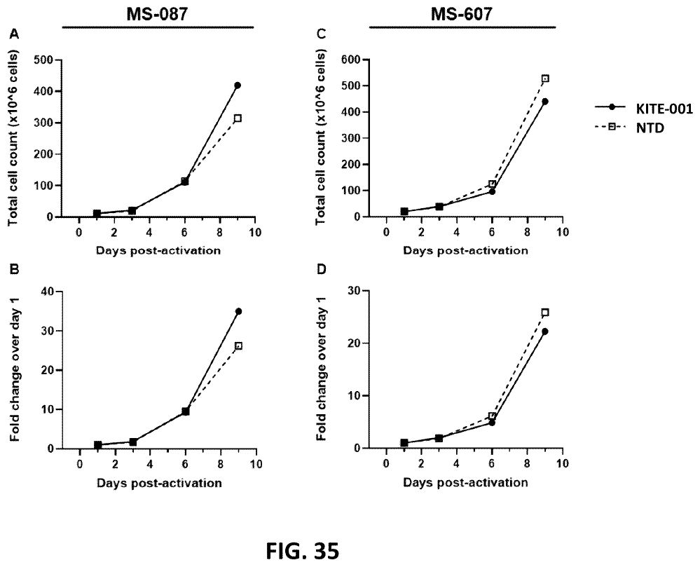

During manufacture of KITE-001 products, T cells were counted and the average total cell count (FIGS. 35A and C) and fold change in cell count compared with Day 1 (day of transduction; FIGS. 35B and D) were calculated. From Days 1 to 9, MS-087 KITE-001 product expanded 35-fold, whereas the corresponding NTD product expanded 26-fold. MS-607 KITE-001 product expanded 22-fold, whereas the corresponding NTD product expanded 26-fold.

### Transduction Efficiency of T-Cell Products

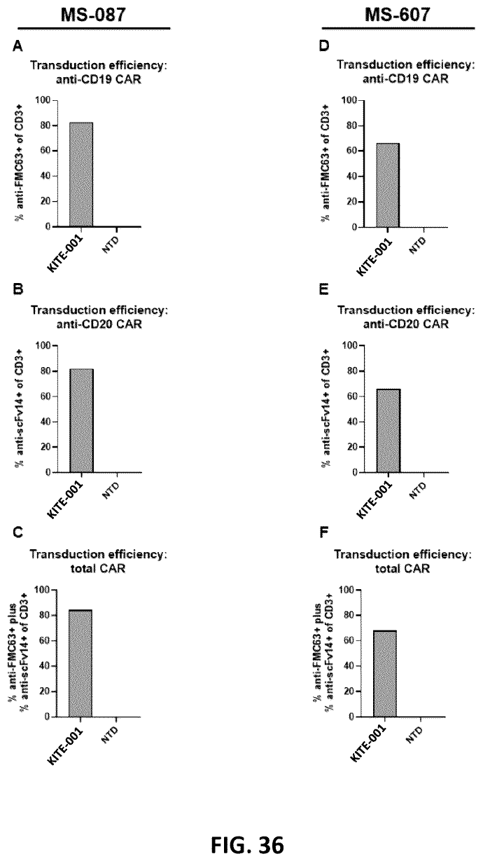

CAR transduction efficiency was evaluated using samples of T-cell products on Day 9 (harvest day). Individual expression of the anti-CD19 CAR in the KITE-001 products based on anti-FMC63 staining was 83% for MS-087 and 67% for MS-607 (FIGS. 36A and D), and 82% and 66% respectively, for the anti-CD20 CAR detected by anti-scFv14 staining in the same samples (FIGS. 36B and E). Total CAR expression of KITE-001 products, as measured by the sum of total anti-CD19 and anti-CD20 CAR expression (calculated by the sum of cells that are % anti-FMC63+ and/or % anti-scFv14+), was 84% and 68% for MS-087 and MS-607, respectively (FIGS. 36C and F). In contrast, NTD control T cell samples did not stain positively for either anti-CD19 or anti-CD20 CAR expression.

### Phenotype of T-Cell Products

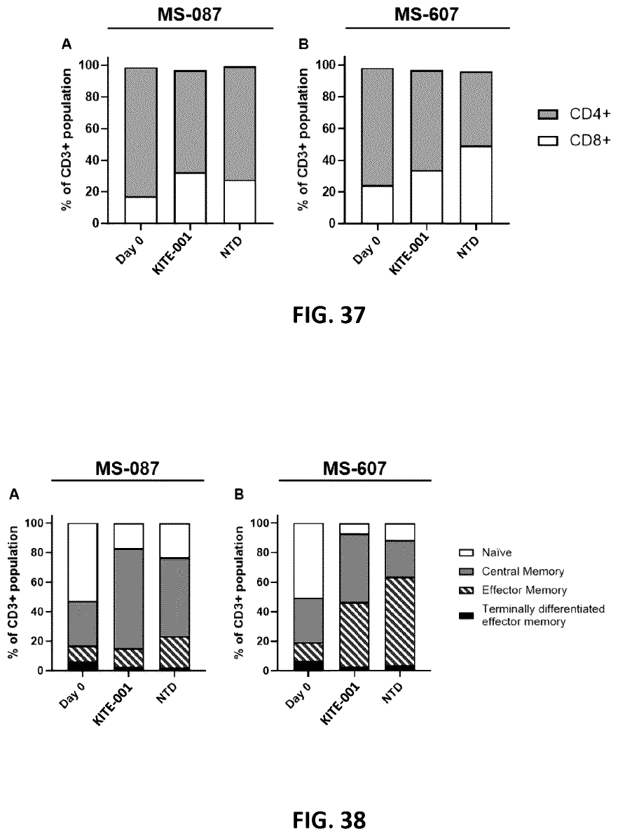

In addition to CAR transduction efficiency, the proportions of CD4⁺ and CD8⁺ T cells were tracked between Days 0 and 9 (FIG. 37). In all T-cell products and as a result of the manufacturing process used for the generation of the T-cell products, an enrichment in the CD8⁺ T-cell population was observed (compared to Day 0).

The proportions of T-cell memory and effector subsets (naive, central memory, effector memory, and terminally differentiated effector memory), as determined by differential CD45RA and CCR7 expression, were assessed in T-cell products between Days 0 and 9 (FIG. 38). Compared with Day 0, an enrichment in the central memory subset and/or effector memory subset was observed for all T-cell products for MS-087 and MS-607 by Day 9.

KITE-001 CAR T-cell products transduced with a bicistronic LVV encoding an anti-CD19 CAR and an anti-CD20 CAR were successfully generated from CD4⁺ and CD8⁺ T cells that had been positively selected from apheresis material from 2 patients with MS. The KITE-001 T-cell products increased in diameter upon activation, expanded, and maintained high viability throughout the manufacturing process. Flow cytometry-based analysis on Day 9 (harvest day) demonstrated that the T cells expressed both the anti-CD19 CAR and anti-CD20 CAR and that total transduction efficiency was 84% and 68% for MS-087 and MS-607 KITE-001 products respectively.

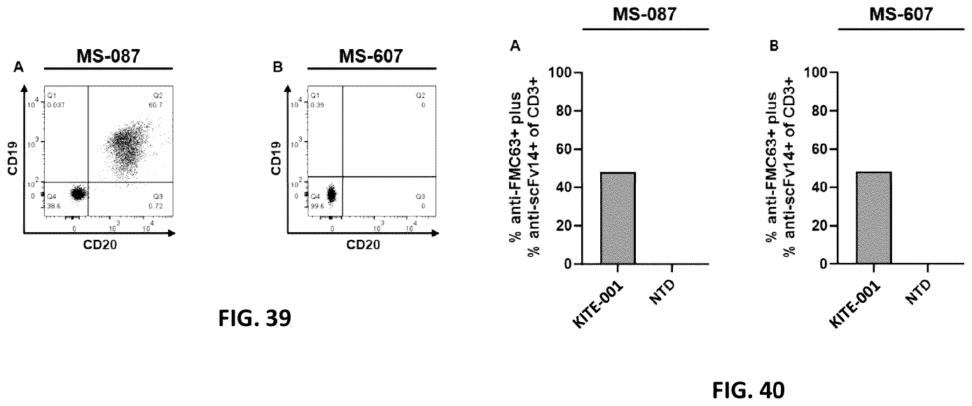

Antigen expression on autologous B cells from patients with MS was evaluated by flow cytometry using commercially available fluorophore-conjugated antibodies. CD19⁺CD20⁺ B cells were detected in MS-087 (FIG. 39), however minimal CD19⁺CD20⁺ B cells were detected in MS-607 likely due to the patient's ocrelizumab (anti-CD20 monoclonal antibody) treatment.

### Total CAR Expression

Total CAR expression between both KITE-001 T-cell products were made comparable by addition of patient-specific NTD T cells. Immediately prior to co-culture setup, total CAR expression was assessed at 48% for MS-087 and 49% for MS-607 (FIGS. 40A and B).

### Cytotoxicity

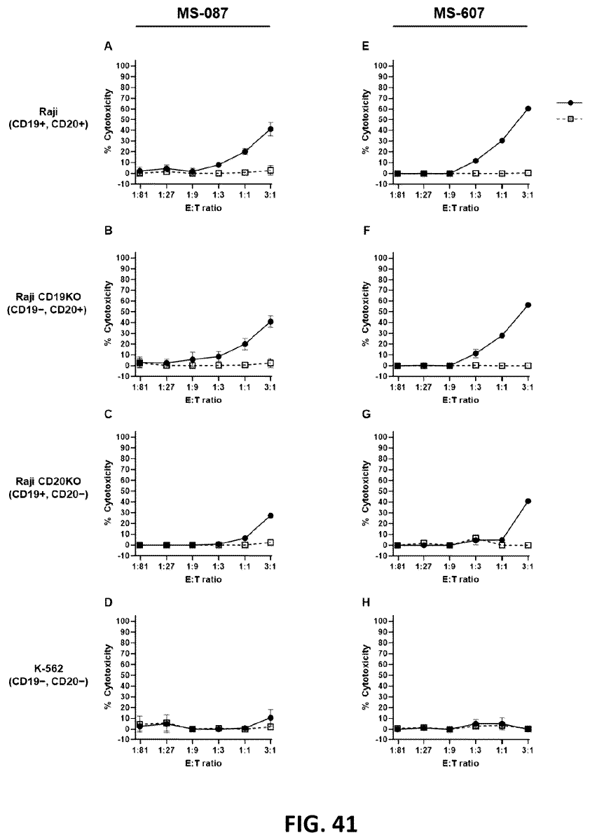

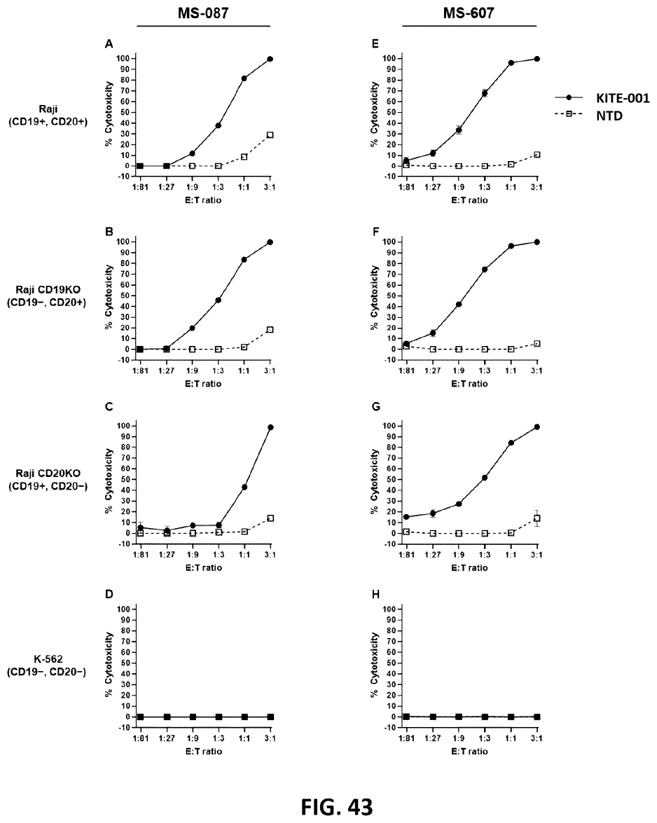

Through a luciferase-based killing assay, KITE-001 products generated from patients with MS demonstrated dose (E:T Ratio)-dependent cytotoxic activity against dual and single antigen-positive Raji target cells beginning at Day 1 (FIGS. 41A-C and E-G) and reaching maximum cytotoxic activity at the highest E:T ratio (3:1) by Day 4 of co-culture (FIGS. 41A-C and E-G). In contrast, NTD control T cells were nonreactive against all the antigen-expressing target cells on Day 1, and demonstrated minimal levels of antigen-independent activity against Raji target cells at higher E:T ratios on Day 4 (FIGS. 41 and 43). Co-culture with K-562 cells showed lack of cytotoxicity in the absence of both antigens at Day 1 and Day 4 (FIGS. 41 and 43D and H).

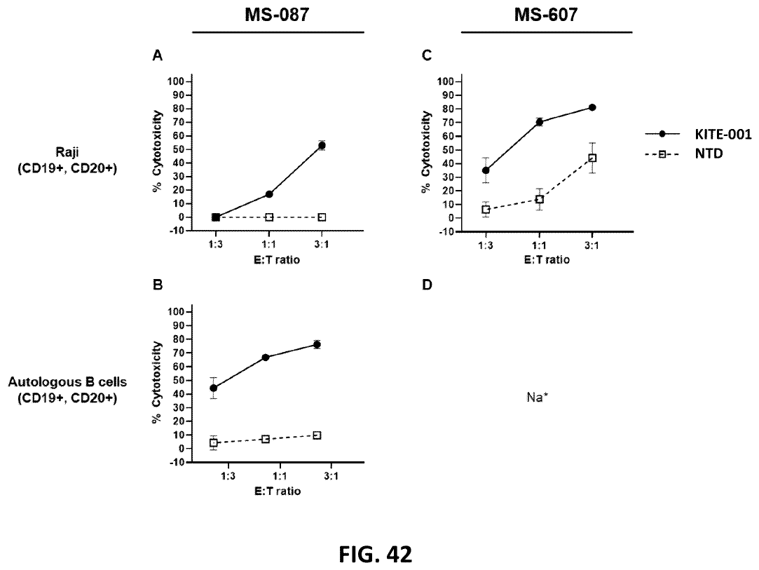

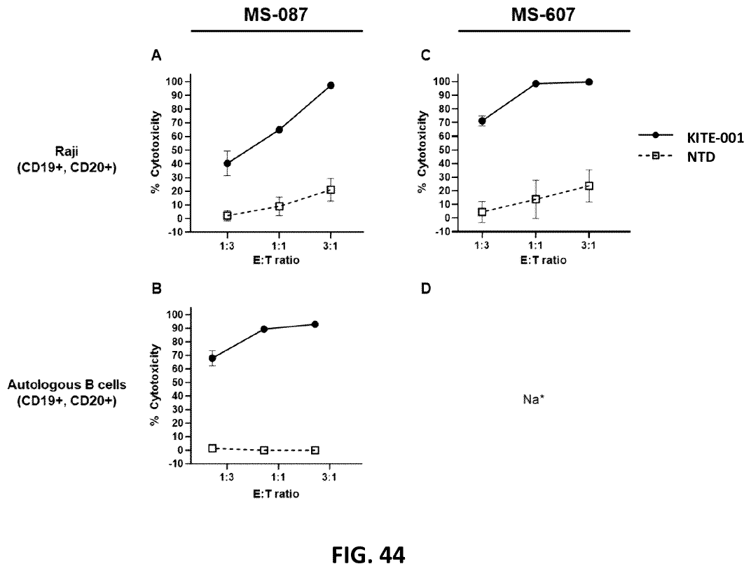

Through a flow cytometry-based killing assay, KITE-001 products from MS-087 exhibited dose (E:T ratio)-dependent specific cytotoxicity against autologous B cells beginning at Day 1 (FIG. 42B), which reached maximum cytotoxic activity at the highest E:T ratio by Day 4 of co-culture (FIG. 44B). Minimal to no cytotoxicity was observed with NTD control T cells on Days 1 and 4 (FIGS. 42 and 44B). To verify the robustness of detecting cytotoxicity of target cells by flow cytometry, Raji cells were used as a control to demonstrate that cytotoxicity of Raji cells from both the luciferase and flow cytometry-based killing assays were dose-dependent. Combined, these results demonstrate that KITE-001 products from patients with MS exhibit robust antigen-dependent and antigen-specific cytotoxicity in co-culture with target cells, including autologous B cells.

### Cytokine Production

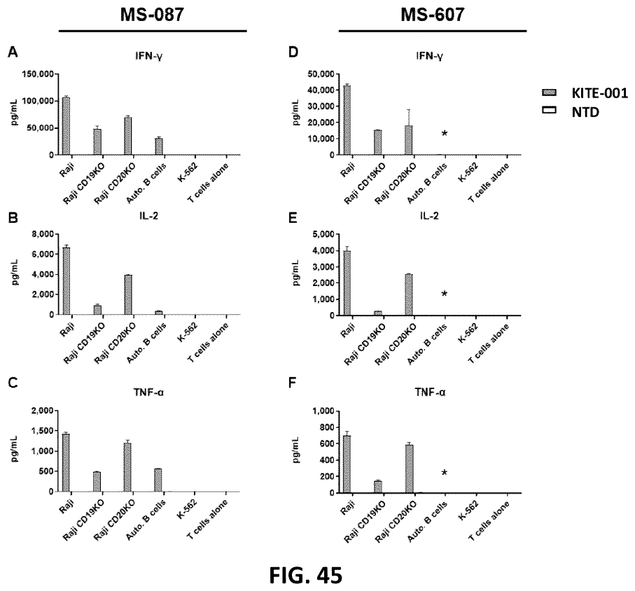

KITE-001 T-cell products yielded measurable levels of IFN-γ, IL-2, and TNF-α pro-inflammatory cytokines with stimulation by CD19 only, CD20 only, or CD19/CD20 expressing target cell lines. Importantly, KITE-001 product from MS-087 also exhibited cytokine production in response to autologous B cells. In contrast, cytokine production was minimal or absent when NTD T cells were co-cultured with any target cells (FIG. 45). Pro-inflammatory cytokine production was specific, as no cytokine production was detected in the absence of target cells (i.e., T cells alone) and there was no cytokine production from KITE-363 T-cell products co-cultured with antigen-negative K-562 (CD19⁻CD20⁻) cells, as shown (FIG. 45). Therefore, these results demonstrate that KITE-001 products from patients with MS showed antigen-dependent and antigen-specific production of IFN-γ, IL-2, and TNF-α in co-culture with target cells, including autologous B cells.

### Proliferation

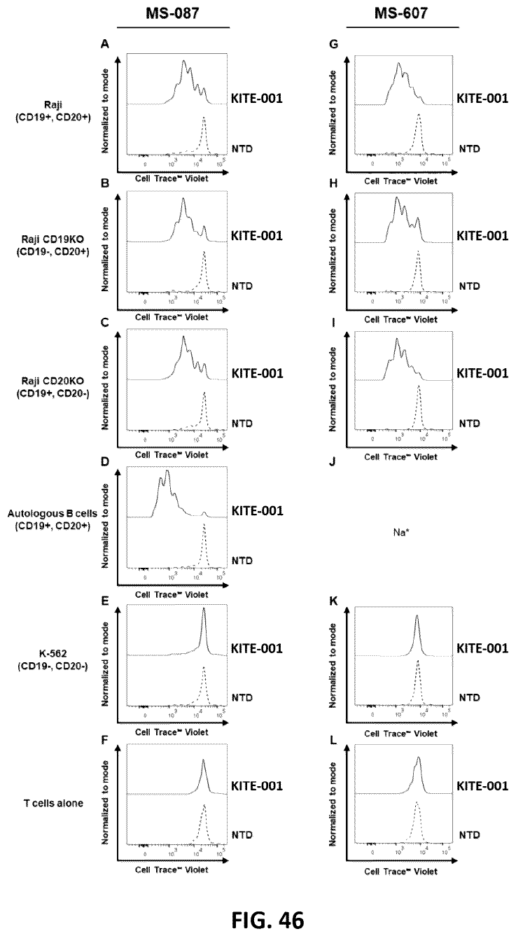

KITE-001 products from patients with MS showed robust proliferation when co-cultured for 4 days with single antigen (Raji CD19KO and Raji CD20KO) and dual antigen-expressing (Raji) target cell lines (FIGS. 46A-C and G-I), but not with antigen-negative K-562 cells (FIGS. 46E and K), indicating target-specific proliferation. Importantly, KITE-001 product from MS-087 exhibited robust proliferation in response to autologous B cells (FIG. 46D). Furthermore, NTD cells displayed minimal to no non-specific proliferation across all co-culture conditions (FIGS. 46A-D and G-J), and T cells cultured alone showed no proliferation (FIGS. 46F and L). The CTV MFI of KITE-001 T-cell products was at least 1 log-fold lower when co-cultured with antigen-positive target cells demonstrating antigen-specific proliferation. Overall, these results demonstrate that KITE-001 products from patients with MS showed robust antigen-dependent and antigen-specific proliferation from co-culture with antigen-positive target cells, including autologous B cells.

In this example, KITE-001 products generated from patients with MS exhibited in vitro activity when challenged with CD19 and/or CD20 antigen-expressing target cells. In vitro assays demonstrated CAR and antigen-specific T-cell functionality: the CAR T-cell products exhibited cytokine production, proliferation, and dose-dependent cytotoxicity against autologous primary B cells and cell lines expressing either CD19 and/or CD20 antigens, but not against cell lines that did not express either antigen. NTD products lacking CAR expression did not react with any of the target cell lines or autologous B cells. These results support antigen-specificity and B-cell depleting activity of KITE-001 manufactured from patients with MS.

### Example 12. Functional Characterization of KITE-001 Manufactured from Patients with Rheumatoid Arthritis

This example assessed manufacturability of KITE-001 from T cells isolated from apheresis material of two patients with rheumatoid arthritis (RA-736 and RA-985). T-cell harvesting, activation, transduction, expansion and evaluation were conducted similar to Example 10.

### Results

### T-Cell Product Viability

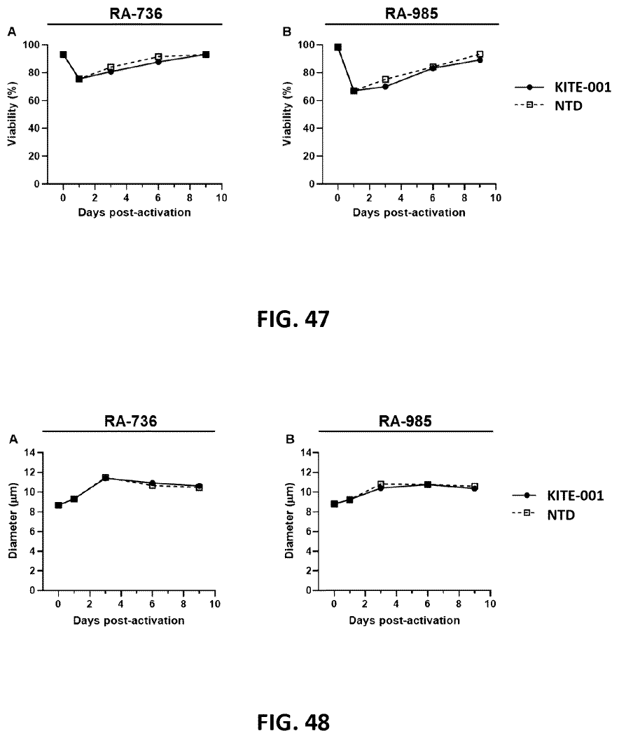

The viability of the T-cell products was assessed from Days 0 to 9. Cell viability was excellent for both T-cell products (FIG. 47) between Days 0 and 9.

### T-Cell Product Activation (Cell Diameter)

Cell diameter was measured for all T-cell products at multiple time points between Days 0 and 9 (FIG. 48) as an indicator of T-cell activation. Cell blasting was observed upon activation for all T-cell products, as evidenced by an increase in cell diameter between Days 1 and 3. The diameter of each patient's T-cell products were comparable on the days they were measured during the manufacturing process.

### T-Cell Product Expansion

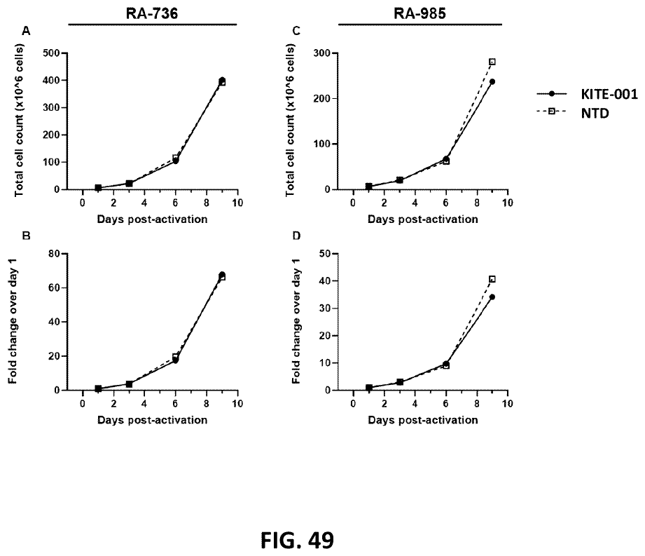

During manufacture of KITE-001 products, T cells were counted and the average total cell count (FIGS. 49A and C) and fold change in cell count compared with Day 1 (day of transduction; FIGS. 49B and D) were calculated. From Days 1 to 9, both KITE-001 products expanded over 30-fold.

### Transduction Efficiency of T-Cell Products

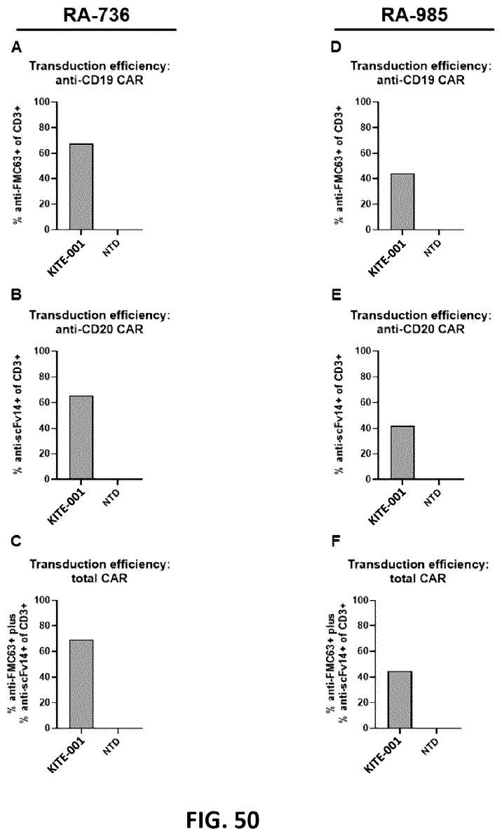

CAR transduction efficiency was evaluated using samples of T-cell products on Day 9 (harvest day). Individual expression of the anti-CD19 CAR in the KITE-001 products based on anti-FMC63 staining (FIGS. 50A and D), and for the anti-CD20 CAR detected by anti-scFv14 staining in the same samples (FIGS. 50B and E). Total CAR expression of KITE-001 products, as measured by the sum of total anti-CD19 and anti-CD20 CAR expression (calculated by the sum of cells that are % anti-FMC63+ and/or % anti-scFv14+), was high (FIGS. 50C and F). In contrast, NTD control T cell samples did not stain positively for either anti-CD19 or anti-CD20 CAR expression.

### Phenotype of T-Cell Products

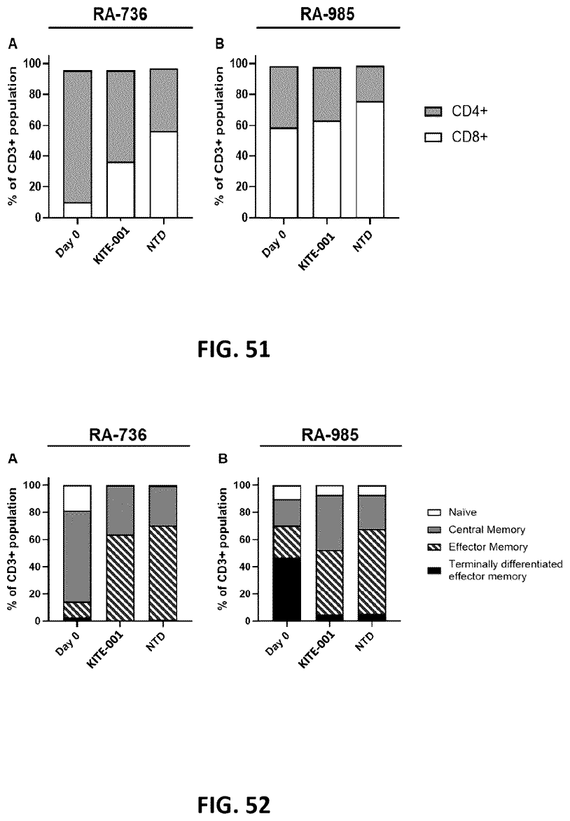

In addition to CAR transduction efficiency, the proportions of CD4⁺ and CD8⁺ T cells were tracked between Days 0 and 9 (FIG. 51). In all T-cell products and as a result of the manufacturing process used for the generation of the T-cell products, an enrichment in the CD8⁺ T-cell population was observed (compared to Day 0).

The proportions of T-cell memory and effector subsets (naive, central memory, effector memory, and terminally differentiated effector memory), as determined by differential CD45RA and CCR7 expression, were assessed in T-cell products between Days 0 and 9 (FIG. 52). Compared with Day 0, an enrichment in the central memory subset and/or effector memory subset was observed for all T-cell products for RA-736 and RA-985 by Day 9.

KITE-001 CAR T-cell products transduced with a bicistronic LVV encoding an anti-CD19 CAR and an anti-CD20 CAR were successfully generated from CD4⁺ and CD8⁺ T cells that had been positively selected from apheresis material from 2 patients with RA. The KITE-001 T-cell products increased in diameter upon activation, expanded, and maintained high viability throughout the manufacturing process. Flow cytometry-based analysis on Day 9 (harvest day) demonstrated that the T cells expressed both the anti-CD19 CAR and anti-CD20 CAR and that total transduction efficiency was high for both RA-736 and RA-985 KITE-001 products.

### Total CAR Expression

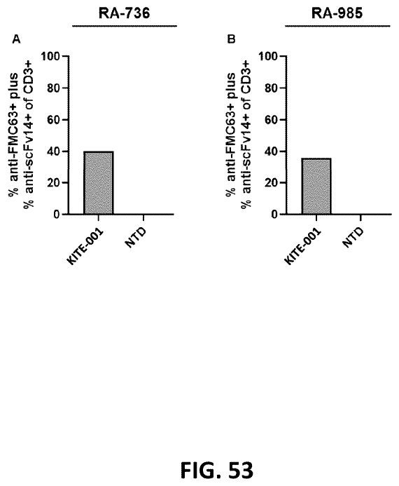

Total CAR expression between both KITE-001 T-cell products were made comparable by addition of patient-specific NTD T cells. Immediately prior to co-culture setup, total CAR expression was assessed at 40% for RA-736 and 38% for RA-985 (FIGS. 53A and B).

### Cytotoxicity

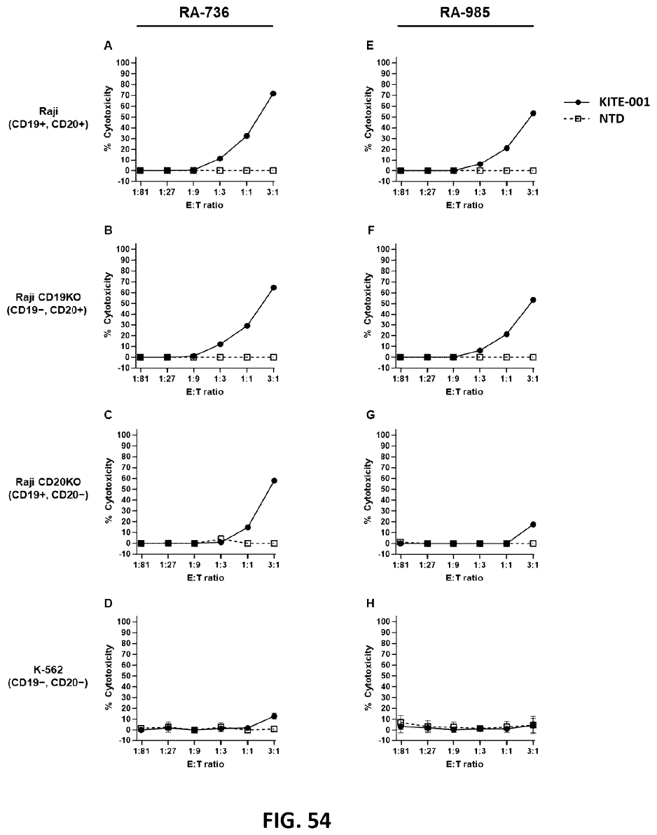

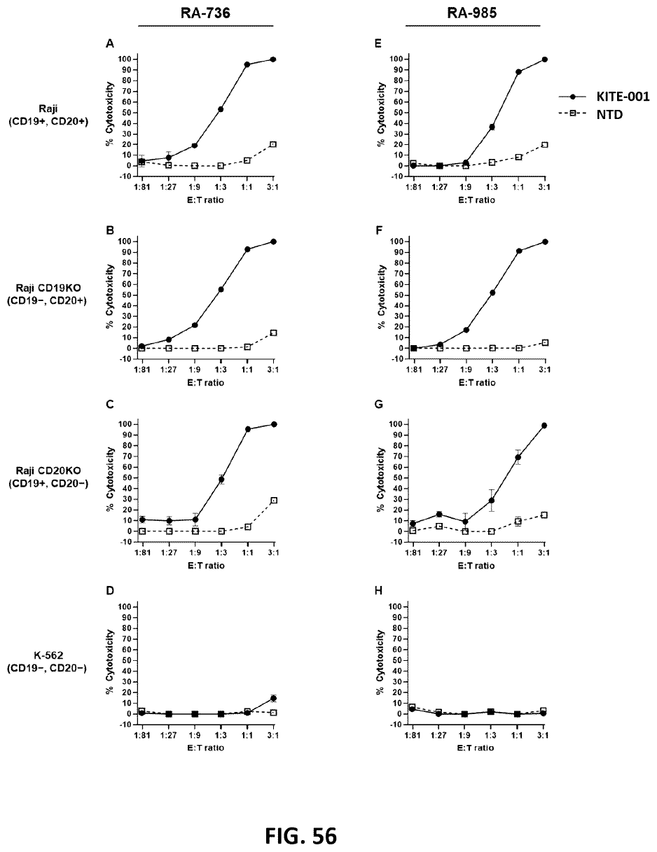

Through a luciferase-based killing assay, KITE-001 products generated from patients with RA demonstrated dose (E:T Ratio)-dependent cytotoxic activity against dual and single antigen-positive Raji target cells beginning at Day 1 (FIGS. 54A-C and E-G) and reaching maximum cytotoxic activity at the highest E:T ratio (3:1) by Day 4 of co-culture (FIGS. 54A-C and E-G). In contrast, NTD control T cells were nonreactive against all the antigen-expressing target cells on Day 1, and demonstrated minimal levels of antigen-independent activity against Raji target cells at higher E:T ratios on Day 4 (FIGS. 54 and 56). Co-culture with K-562 cells showed lack of cytotoxicity in the absence of both antigens at Day 1 and Day 4 (FIGS. 54 and 56D and H).

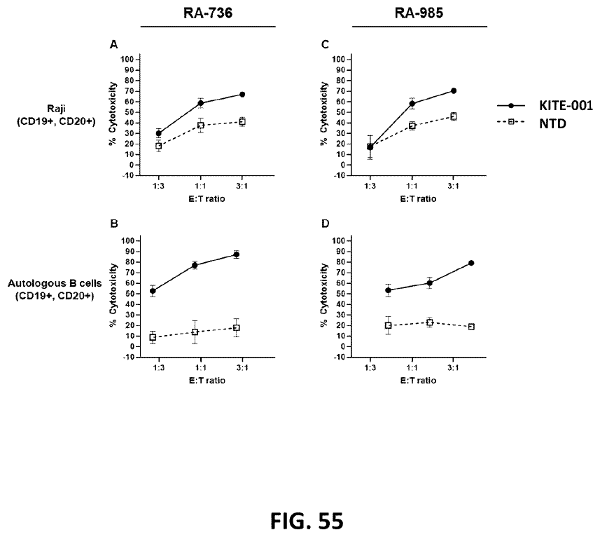

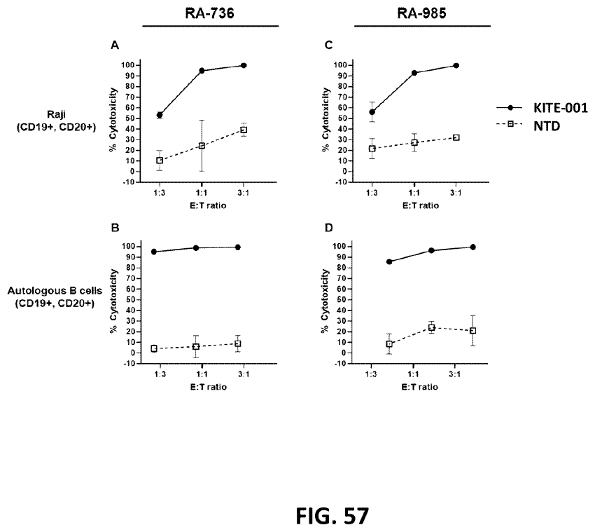

Through a flow cytometry-based killing assay, KITE-001 products exhibited dose (E:T ratio)-dependent specific cytotoxicity against autologous B cells beginning at Day 1 (FIGS. 55B and D), which reached maximum cytotoxic activity at the highest E:T ratio by Day 4 of co-culture (FIGS. 57B and D). Minimal to no cytotoxicity was observed with NTD control T cells on Days 1 and 4 (FIGS. 55 and 57). To verify the robustness of detecting cytotoxicity of target cells by flow cytometry, Raji cells were used as a control to demonstrate that cytotoxicity of Raji cells from both the luciferase and flow cytometry-based killing assays were dose-dependent. Combined, these results demonstrate that KITE-001 products from patients with RA exhibit robust antigen-dependent and antigen-specific cytotoxicity in co-culture with target cells, including autologous B cells.

### Cytokine Production

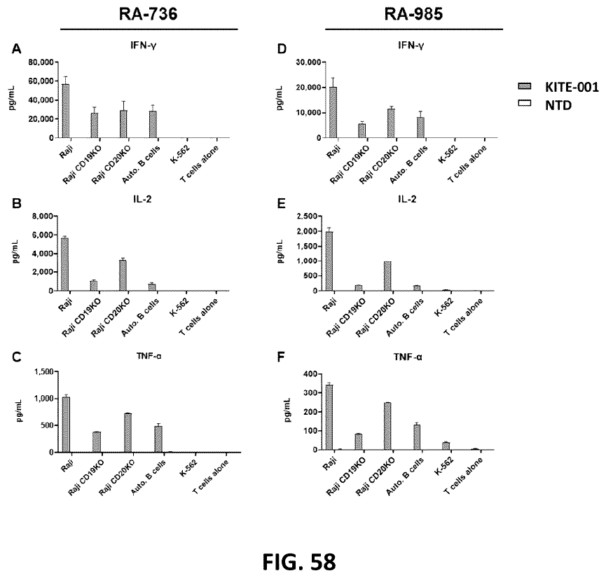

KITE-001 T-cell products yielded measurable levels of IFN-γ, IL-2, and TNF-α pro-inflammatory cytokines with stimulation by CD19 only, CD20 only, or CD19/CD20 expressing target cell lines. Importantly, KITE-001 product from RA-736 also exhibited cytokine production in response to autologous B cells. In contrast, cytokine production was minimal or absent when NTD T cells were co-cultured with any target cells (FIG. 58). Pro-inflammatory cytokine production was specific, as no cytokine production was detected in the absence of target cells (i.e., T cells alone) and there was no cytokine production from KITE-363 T-cell products co-cultured with antigen-negative K-562 (CD19⁻CD20⁻) cells, as shown (FIG. 58). Therefore, these results demonstrate that KITE-001 products from patients with RA showed antigen-dependent and antigen-specific production of IFN-γ, IL-2, and TNF-α in co-culture with target cells, including autologous B cells.

### Proliferation

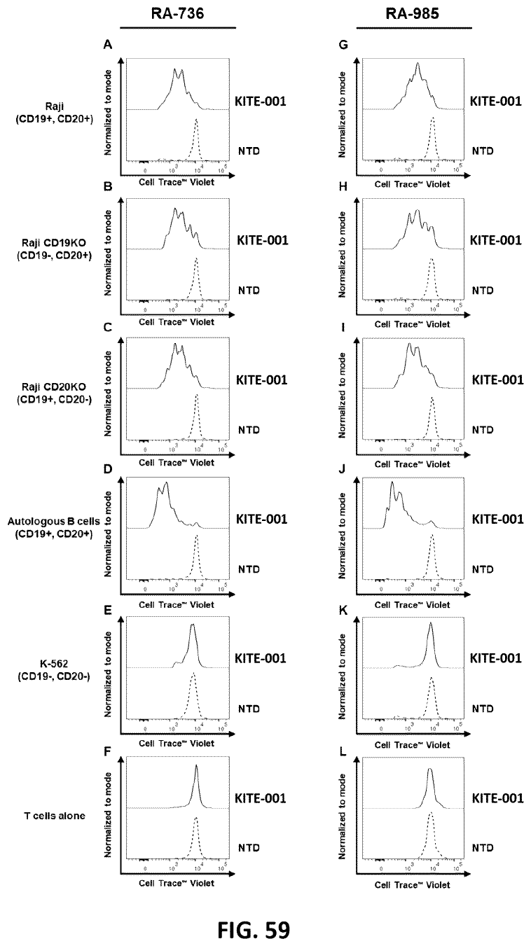

KITE-001 products from patients with RA showed robust proliferation when co-cultured for 4 days with single antigen (Raji CD19KO and Raji CD20KO) and dual antigen-expressing (Raji) target cell lines (FIGS. 59A-C and G-I), but not with antigen-negative K-562 cells (FIGS. 59E and K), indicating target-specific proliferation. Importantly, KITE-001 product from RA-736 exhibited robust proliferation in response to autologous B cells (FIG. 59D). Furthermore, NTD cells displayed minimal to no non-specific proliferation across all co-culture conditions (FIGS. 59A-D and G-J), and T cells cultured alone showed no proliferation (FIGS. 59F and L). The CTV MFI of KITE-001 T-cell products was significantly lower when co-cultured with antigen-positive target cells demonstrating antigen-specific proliferation. Overall, these results demonstrate that KITE-001 products from patients with RA showed robust antigen-dependent and antigen-specific proliferation from co-culture with antigen-positive target cells, including autologous B cells.

In this example, KITE-001 products generated from patients with RA exhibited in vitro activity when challenged with CD19 and/or CD20 antigen-expressing target cells. In vitro assays demonstrated CAR and antigen-specific T-cell functionality: the CAR T-cell products exhibited cytokine production, proliferation, and dose-dependent cytotoxicity against autologous primary B cells and cell lines expressing either CD19 and/or CD20 antigens, but not against cell lines that did not express either antigen. NTD products lacking CAR expression did not react with any of the target cell lines or autologous B cells. These results support antigen-specificity and B-cell depleting activity of KITE-001 manufactured from patients with RA.

### Example 13. Functional Characterization of KITE-001 Manufactured from Patients with Sjögren's Syndrome

This example assessed manufacturability of KITE-001 from T cells isolated from apheresis material of two patients with Sjogren's Syndrome (SJ-167 and SJ-663). T-cell harvesting, activation, transduction, expansion and evaluation were conducted similar to Example 10.

### Results

### T-Cell Product Viability

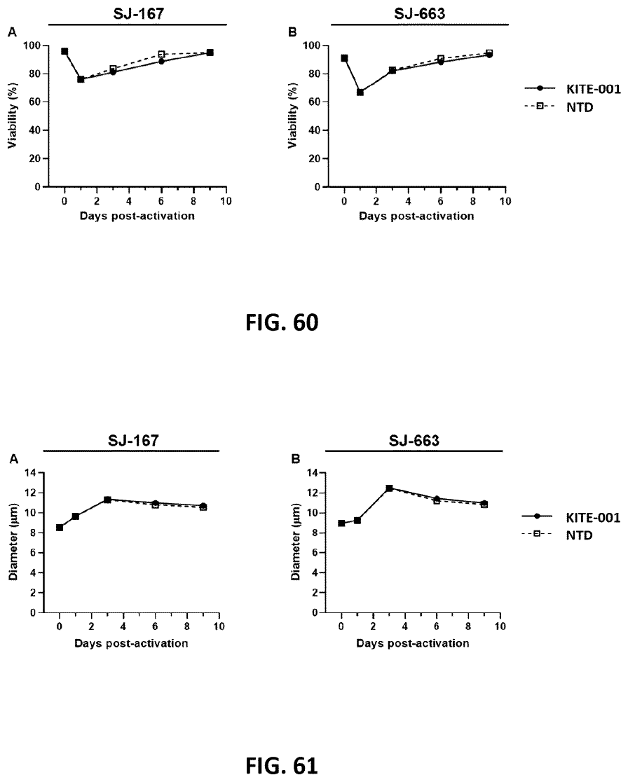

The viability of the T-cell products was assessed from Days 0 to 9. Cell viability was excellent for both T-cell products (FIG. 60) between Days 0 and 9.

### T-Cell Product Activation (Cell Diameter)

Cell diameter was measured for all T-cell products at multiple time points between Days 0 and 9 (FIG. 61) as an indicator of T-cell activation. Cell blasting was observed upon activation for all T-cell products, as evidenced by an increase in cell diameter between Days 1 and 3. The diameter of each patient's T-cell products were comparable on the days they were measured during the manufacturing process.

### T-Cell Product Expansion

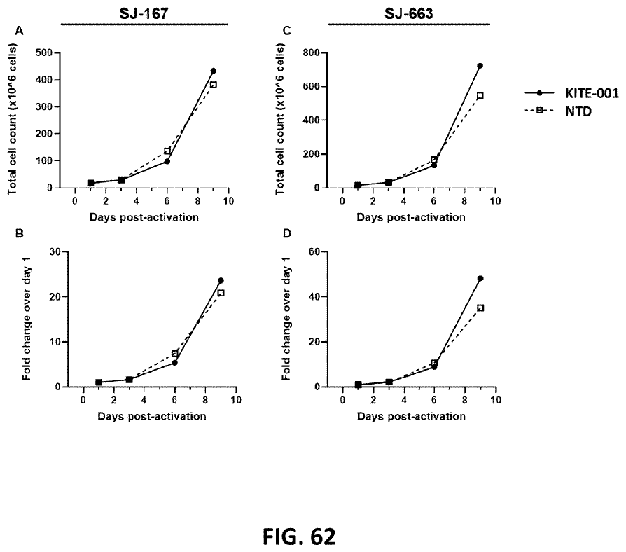

During manufacture of KITE-001 products, T cells were counted and the average total cell count (FIGS. 62A and C) and fold change in cell count compared with Day 1 (day of transduction; FIGS. 62B and D) were calculated. From Days 1 to 9, both KITE-001 products expanded over 30-fold.

### Transduction Efficiency of T-Cell Products

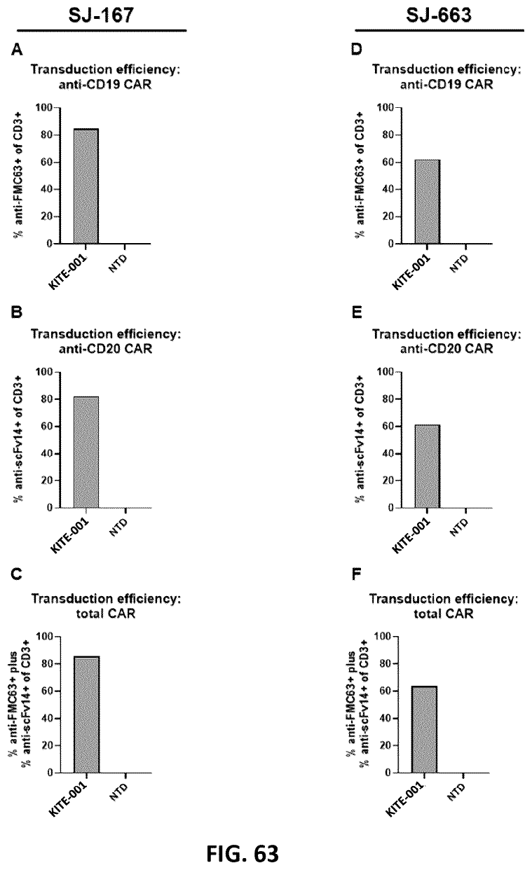

CAR transduction efficiency was evaluated using samples of T-cell products on Day 9 (harvest day). Individual expression of the anti-CD19 CAR in the KITE-001 products based on anti-FMC63 staining (FIGS. 63A and D), and for the anti-CD20 CAR detected by anti-scFv14 staining in the same samples (FIGS. 63B and E). Total CAR expression of KITE-001 products, as measured by the sum of total anti-CD19 and anti-CD20 CAR expression (calculated by the sum of cells that are % anti-FMC63+ and/or % anti-scFv14+), was high (FIGS. 63C and F). In contrast, NTD control T cell samples did not stain positively for either anti-CD19 or anti-CD20 CAR expression.

### Phenotype of T-Cell Products

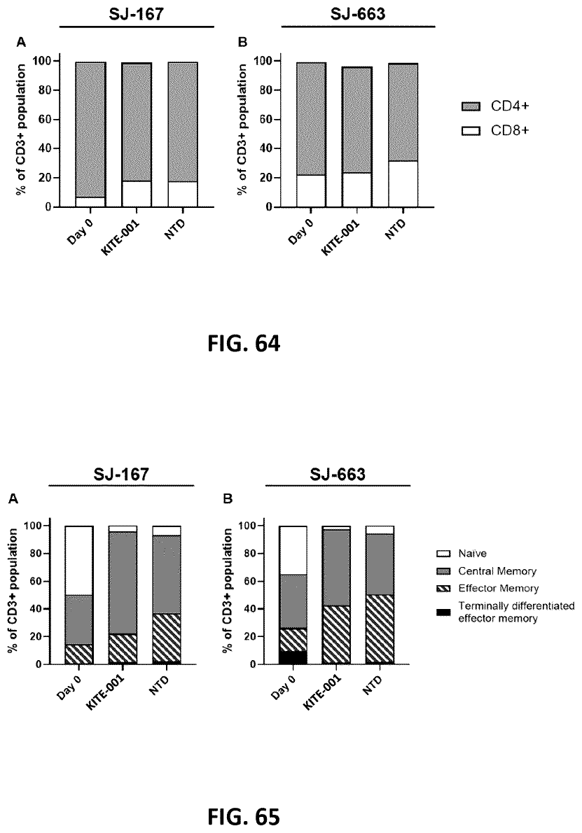

In addition to CAR transduction efficiency, the proportions of CD4⁺ and CD8⁺ T cells were tracked between Days 0 and 9 (FIG. 64). In all T-cell products and as a result of the manufacturing process used for the generation of the T-cell products, an enrichment in the CD8⁺ T-cell population was observed (compared to Day 0).

The proportions of T-cell memory and effector subsets (naive, central memory, effector memory, and terminally differentiated effector memory), as determined by differential CD45SJ and CCR7 expression, were assessed in T-cell products between Days 0 and 9 (FIG. 65). Compared with Day 0, an enrichment in the central memory subset and/or effector memory subset was observed for all T-cell products for SJ-167 and SJ-663 by Day 9.

KITE-001 CAR T-cell products transduced with a bicistronic LVV encoding an anti-CD19 CAR and an anti-CD20 CAR were successfully generated from CD4⁺ and CD8⁺ T cells that had been positively selected from apheresis material from 2 patients with SJ. The KITE-001 T-cell products increased in diameter upon activation, expanded, and maintained high viability throughout the manufacturing process. Flow cytometry-based analysis on Day 9 (harvest day) demonstrated that the T cells expressed both the anti-CD19 CAR and anti-CD20 CAR and that total transduction efficiency was high for both SJ-167 and SJ-663 KITE-001 products.

### Total CAR Expression

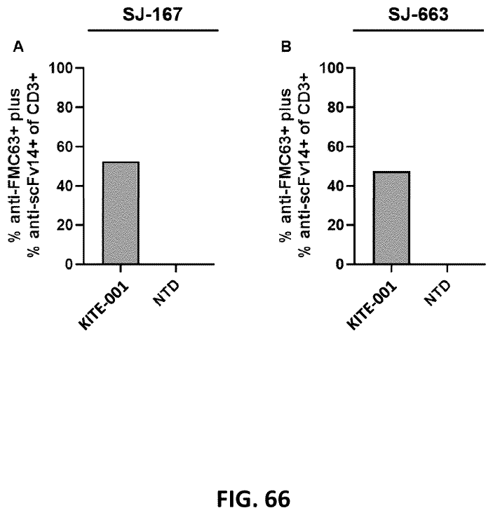

Total CAR expression between both KITE-001 T-cell products were made comparable by addition of patient-specific NTD T cells. Immediately prior to co-culture setup, total CAR expression was assessed at 52% for SJ-167 and 45% for SJ-663 (FIGS. 66A and B).

### Cytotoxicity

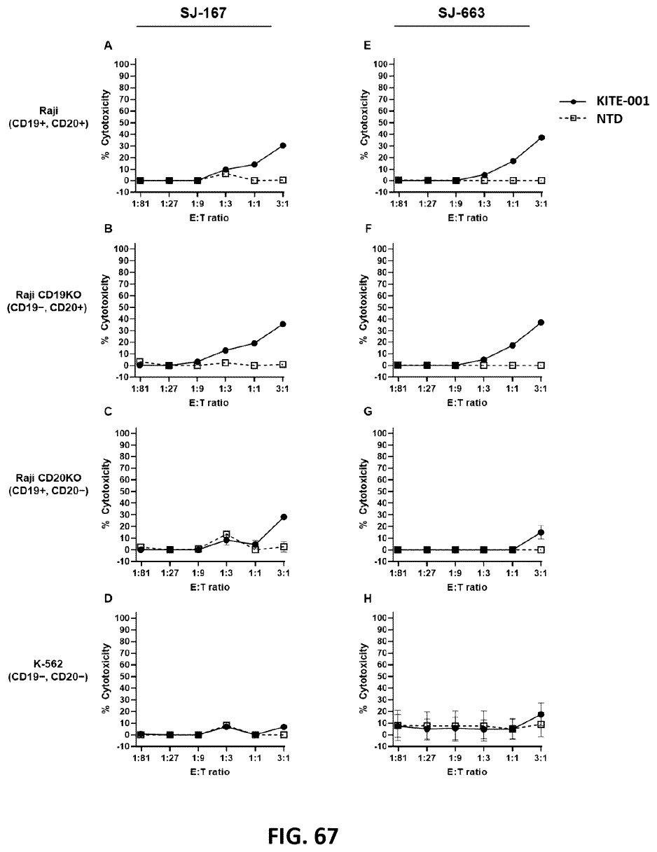

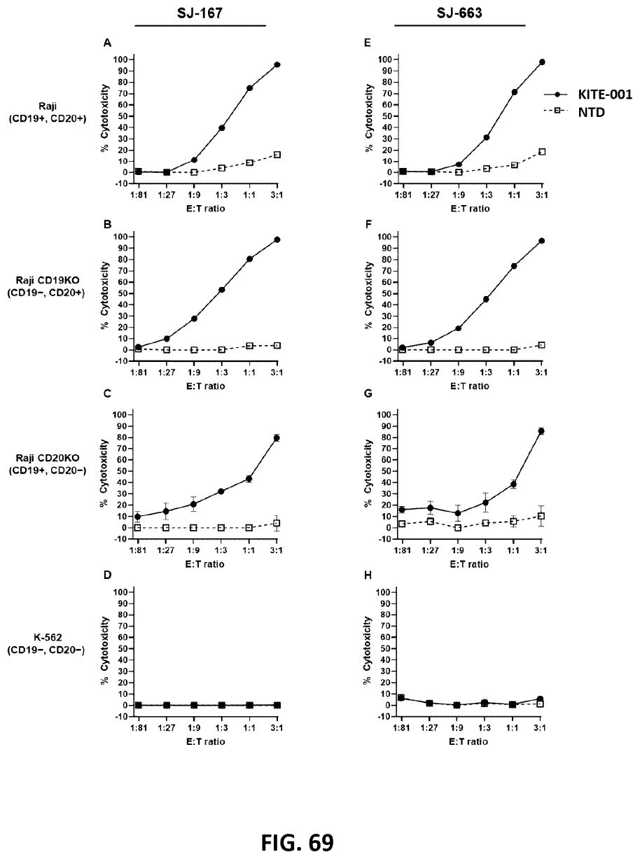

Through a luciferase-based killing assay, KITE-001 products generated from patients with SJ demonstrated dose (E:T Ratio)-dependent cytotoxic activity against dual and single antigen-positive Raji target cells beginning at Day 1 (FIGS. 67A-C and E-G) and reaching maximum cytotoxic activity at the highest E:T ratio (3:1) by Day 4 of co-culture (FIGS. 67A-C and E-G). In contrast, NTD control T cells were nonreactive against all the antigen-expressing target cells on Day 1, and demonstrated minimal levels of antigen-independent activity against Raji target cells at higher E:T ratios on Day 4 (FIGS. 67 and 69). Co-culture with K-562 cells showed lack of cytotoxicity in the absence of both antigens at Day 1 and Day 4 (FIGS. 67 and 69D and H).

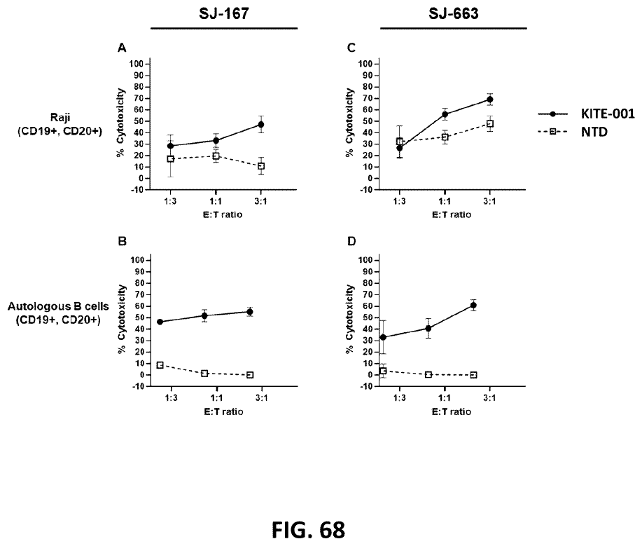

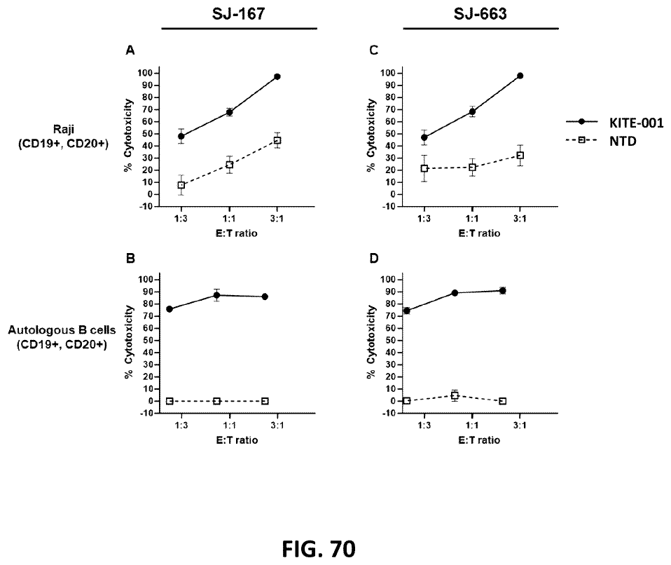

Through a flow cytometry-based killing assay, KITE-001 products exhibited dose (E:T ratio)-dependent specific cytotoxicity against autologous B cells beginning at Day 1 (FIGS. 68B and D), which reached maximum cytotoxic activity at the highest E:T ratio by Day 4 of co-culture (FIGS. 70B and D). Minimal to no cytotoxicity was observed with NTD control T cells on Days 1 and 4 (FIGS. 68 and 70). To verify the robustness of detecting cytotoxicity of target cells by flow cytometry, Raji cells were used as a control to demonstrate that cytotoxicity of Raji cells from both the luciferase and flow cytometry-based killing assays were dose-dependent. Combined, these results demonstrate that KITE-001 products from patients with SJ exhibit robust antigen-dependent and antigen-specific cytotoxicity in co-culture with target cells, including autologous B cells.

### Cytokine Production

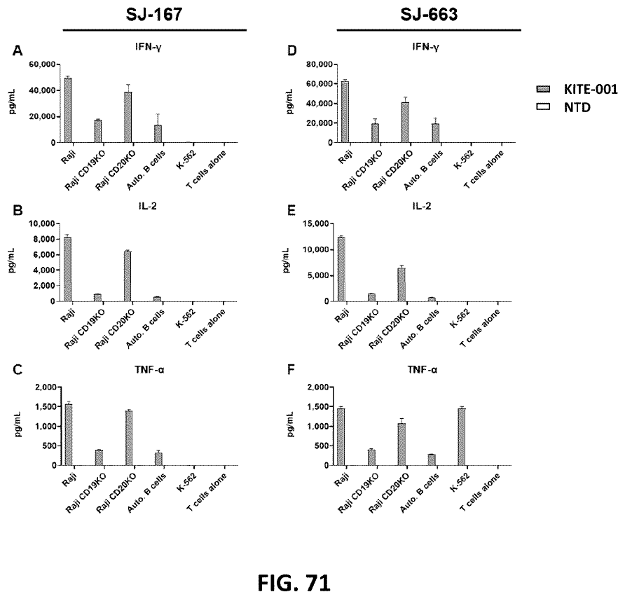

KITE-001 T-cell products yielded measurable levels of IFN-γ, IL-2, and TNF-α pro-inflammatory cytokines with stimulation by CD19 only, CD20 only, or CD19/CD20 expressing target cell lines. Importantly, KITE-001 product from SJ-167 also exhibited cytokine production in response to autologous B cells. In contrast, cytokine production was minimal or absent when NTD T cells were co-cultured with any target cells (FIG. 71). Pro-inflammatory cytokine production was specific, as no cytokine production was detected in the absence of target cells (i.e., T cells alone) and there was no cytokine production from KITE-363 T-cell products co-cultured with antigen-negative K-562 (CD19⁻ CD20⁻) cells, as shown (FIG. 71). Therefore, these results demonstrate that KITE-001 products from patients with SJ showed antigen-dependent and antigen-specific production of IFN-γ, IL-2, and TNF-α in co-culture with target cells, including autologous B cells.

### Proliferation

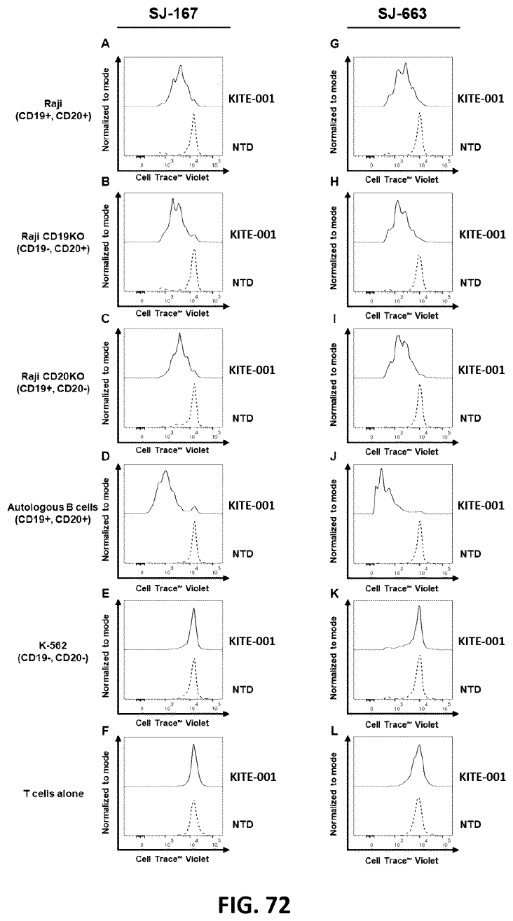

KITE-001 products from patients with SJ showed robust proliferation when co-cultured for 4 days with single antigen (Raji CD19KO and Raji CD20KO) and dual antigen-expressing (Raji) target cell lines (FIGS. 72A-C and G-I), but not with antigen-negative K-562 cells (FIGS. 72E and K), indicating target-specific proliferation. Importantly, KITE-001 product from SJ-167 exhibited robust proliferation in response to autologous B cells (FIG. 72D). Furthermore, NTD cells displayed minimal to no non-specific proliferation across all co-culture conditions (FIGS. 72A-D and G-J), and T cells cultured alone showed no proliferation (FIGS. 72F and L). The CTV MFI of KITE-001 T-cell products was significantly lower when co-cultured with antigen-positive target cells demonstrating antigen-specific proliferation. Overall, these results demonstrate that KITE-001 products from patients with SJ showed robust antigen-dependent and antigen-specific proliferation from co-culture with antigen-positive target cells, including autologous B cells.

In this example, KITE-001 products generated from patients with SJ exhibited in vitro activity when challenged with CD19 and/or CD20 antigen-expressing target cells. In vitro assays demonstrated CAR and antigen-specific T-cell functionality: the CAR T-cell products exhibited cytokine production, proliferation, and dose-dependent cytotoxicity against autologous primary B cells and cell lines expressing either CD19 and/or CD20 antigens, but not against cell lines that did not express either antigen. NTD products lacking CAR expression did not react with any of the target cell lines or autologous B cells. These results support antigen-specificity and B-cell depleting activity of KITE-001 manufactured from patients with SJ.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A method for treating an autoimmune disease or condition in a patient in needed thereof, comprising administering to the patient a T cell comprising an anti-CD19 chimeric antigen receptor (CAR) that specifically binds CD19 and an anti-CD20 CAR that specifically binds CD20, wherein
the anti-CD19 CAR comprises a first antigen binding fragment comprising a heavy chain variable region (VH) comprising complementarity determining regions (CDR) 1, 2, and 3, and a light chain variable region (VL) comprising a CDR1, a CDR2 and a CDR3, wherein the VH CDR1, VH CDR2, VH CDR3, VL CDR1, VL CDR2 and VL CDR3 of the anti-CD19 CAR comprise the amino acid sequences of SEQ ID NO:21-26, respectively, and
the anti-CD20 CAR comprises a second antigen binding fragment comprising a VH comprising a CDR1, a CDR2, and a CDR3, and a VL comprising a CDR1, a CDR2 and a CDR3, wherein the VH CDR1, VH CDR2, VH CDR3, VL CDR1, VL CDR2 and VL CDR3 of the anti-CD20 CAR comprise the amino acid sequences of SEQ ID NO:27-32, respectively.

2. The method of claim 1, wherein the anti-CD19 CAR further comprises a first transmembrane domain and/or a first CD3 zeta signaling domain.

3. The method of claim 1, where the anti-CD20 CAR further comprises a second transmembrane domain and/or a first CD3 zeta signaling domain.

4. The method of claim 1, wherein the anti-CD19 CAR further comprises a first co-stimulatory domain, and the anti-CD20 CAR further comprises a second co-stimulatory domain, wherein the first co-stimulatory domain comprises a CD28 intracellular signaling domain and the second co-stimulatory domain comprises a 4-1BB intracellular signaling domain.

5. The method of claim 4, wherein the CD28 intracellular signaling domain comprises an amino acid sequence that is at least 90% identical to SEQ ID NO:10.

6. The method of claim 4, wherein the 4-1BB intracellular signaling domain comprises an amino acid sequence that is at least 90% identical to SEQ ID NO:17.

7. The method of claim 1, wherein the autoimmune disease or condition is a rheumatological autoimmune disease or condition, or a neurological autoimmune disease or condition.

8. The method of claim 7, wherein the rheumatological autoimmune disease or condition is selected from the group consisting of lupus nephritis (LN), non-renal systemic lupus erythematosus (SLE), systemic sclerosis (SSc), idiopathic inflammatory myopathies (IIM), Anti-neutrophil cytoplasmic antibody-associated (ANCA) vasculitis, Sjogren's syndrome, rheumatoid arthritis (RA), and pediatric lupus.

9. The method of claim 7, wherein the neurological autoimmune disease or condition is selected from the group consisting of myasthenia gravis, multiple sclerosis, and chronic inflammatory demyelinating polyneuropathy.

10. The method of claim 7, wherein the autoimmune disease or condition is relapsed/refractory or moderate to severe, progressive systemic lupus erythematosus (SLE) with or without lupus nephritis (LN), systemic sclerosis (SSc), or idiopathic inflammatory myopathy (IIM).

11. The method of claim 1, wherein the T cell is administered to the patient at a dose of 0.2×10⁶ to 2×10⁶ cells per kilogram of body weight of the patient, but not greater than 1×10⁸ cells.

12. The method of claim 11, wherein the T cell is administered to the patient at a dose of 0.4×10⁶ to 0.6×10⁶ cells per kilogram of body weight of the patient.

13. The method of claim 11, wherein the T cell is administered to the patient at a dose of 0.8×10⁶ to 1.2×10⁶ cells per kilogram of body weight of the patient.

14. The method of claim 11, wherein the T cell is administered no more than once every 6 months.

15.-16. (canceled)

17. The method of claim 1, wherein the VH and VL of the anti-CD19 CAR comprise the amino acid sequences of SEQ ID NO:4 and 5, respectively.

18. (canceled)

19. The method of claim 17, wherein the VH and VL of the anti-CD20 CAR comprise the amino acid sequences of SEQ ID NO:12 and 13, respectively.

20.-29. (canceled)

30. The method of claim 1, wherein the anti-CD19 CAR comprises the amino acid sequence of SEQ ID NO:1 and the anti-CD20 CAR comprises the amino acid sequence of SEQ ID NO:2.

31. The method of claim 1, wherein the T cell comprises a polynucleotide comprising an open reading frame (ORF) that encodes a bicistronic CAR that comprises the anti-CD19 CAR and the anti-CD20 CAR.

32.-35. (canceled)

36. The method of claim 31, wherein the polynucleotide comprises the nucleic acid sequence of SEQ ID NO:39.

37. A T cell comprising an anti-CD19 chimeric antigen receptor (CAR) that specifically binds CD19 and an anti-CD20 CAR that specifically binds CD20, wherein
the anti-CD19 CAR comprises a first antigen binding fragment comprising a heavy chain variable region (VH) comprising complementarity determining regions (CDR) 1, 2, and 3, and a light chain variable region (VL) comprising a CDR1, a CDR2 and a CDR3, wherein the VH CDR1, VH CDR2, VH CDR3, VL CDR1, VL CDR2 and VL CDR3 of the anti-CD19 CAR comprise the amino acid sequences of SEQ ID NO:21-26, respectively, and
the anti-CD20 CAR comprises a second antigen binding fragment comprising a VH comprising a CDR1, a CDR2, and a CDR3, and a VL comprising a CDR1, a CDR2 and a CDR3, wherein the VH CDR1, VH CDR2, VH CDR3, VL CDR1, VL CDR2 and VL CDR3 of the anti-CD20 CAR comprise the amino acid sequences of SEQ ID NO:27-32, respectively.

38.-68. (canceled)

69. A polynucleotide encoding an anti-CD19 chimeric antigen receptor (CAR) that specifically binds CD19 and an anti-CD20 CAR that specifically binds CD20, wherein
the anti-CD19 CAR comprises a first antigen binding fragment comprising a heavy chain variable region (VH) comprising complementarity determining regions (CDR) 1, 2, and 3, and a light chain variable region (VL) comprising a CDR1, a CDR2 and a CDR3, wherein the VH CDR1, VH CDR2, VH CDR3, VL CDR1, VL CDR2 and VL CDR3 of the anti-CD19 CAR comprise the amino acid sequences of SEQ ID NO:21-26, respectively, and
the anti-CD20 CAR comprises a second antigen binding fragment comprising a VH comprising a CDR1, a CDR2, and a CDR3, and a VL comprising a CDR1, a CDR2 and a CDR3, wherein the VH CDR1, VH CDR2, VH CDR3, VL CDR1, VL CDR2 and VL CDR3 of the anti-CD20 CAR comprise the amino acid sequences of SEQ ID NO:27-32, respectively.

70.-95. (canceled)

96. An anti-CD20 chimeric antigen receptor (CAR) that specifically binds CD20, comprising an antigen binding fragment comprising a VH comprising a CDR1, a CDR2, and a CDR3, and a VL comprising a CDR1, a CDR2 and a CDR3, wherein the VH CDR1, VH CDR2, VH CDR3, VL CDR1, VL CDR2 and VL CDR3 of comprise the amino acid sequences of SEQ ID NO:27-32, respectively.

97.-109. (canceled)


SEQUENCE LISTING (39 sequences)

SEQ ID NO: 1 - protein, 467 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 495 aa, synthetic construct
QVQLVQSGAEVKKPGASVKVSCKASGYTFKEYGISWVRQAPGQGLEWMGWISAYSGHTYYAQKLQGRVTMTTDTSTSTAYMELRSLRSDDTAVYYCARGPHYDDWSGFIIWFDPWGQGTLVTVSSGSTSGSGKPGSGEGSTKGDIQMTQSPSSLSASVGDRVTITCRASQSISSYLNWYQQKPGKAPKLLIYAASSLQSGVPSRFSGSGSGTDFTLTISSLQPEDFATYYCQQSYRFPPTFGQGTKVEIKAAAFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 1031 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRSGSGEGRGSLLTCGDVEENPGPMALPVTALLLPLALLLHAARPQVQLVQSGAEVKKPGASVKVSCKASGYTFKEYGISWVRQAPGQGLEWMGWISAYSGHTYYAQKLQGRVTMTTDTSTSTAYMELRSLRSDDTAVYYCARGPHYDDWSGFIIWFDPWGQGTLVTVSSGSTSGSGKPGSGEGSTKGDIQMTQSPSSLSASVGDRVTITCRASQSISSYLNWYQQKPGKAPKLLIYAASSLQSGVPSRFSGSGSGTDFTLTISSLQPEDFATYYCQQSYRFPPTFGQGTKVEIKAAAFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 5 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 6 - protein, 18 aa, synthetic construct
GSTSGSGKPGSGEGSTKG

SEQ ID NO: 7 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 8 - protein, 39 aa, synthetic construct
IEVMYPPPYLDNEKSNGTIIHVKGKHLCPSPLFPGPSKP

SEQ ID NO: 9 - protein, 27 aa, synthetic construct
FWVLVVVGGVLACYSLLVTVAFIIFWV

SEQ ID NO: 10 - protein, 41 aa, synthetic construct
RSKRSRLLHSDYMNMTPRRPGPTRKHYQPYAPPRDFAAYRS

SEQ ID NO: 11 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 125 aa, synthetic construct
QVQLVQSGAEVKKPGASVKVSCKASGYTFKEYGISWVRQAPGQGLEWMGWISAYSGHTYYAQKLQGRVTMTTDTSTSTAYMELRSLRSDDTAVYYCARGPHYDDWSGFIIWFDPWGQGTLVTVSS

SEQ ID NO: 13 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYRFPPTFGQGTKVEIK

SEQ ID NO: 14 - protein, 250 aa, synthetic construct
QVQLVQSGAEVKKPGASVKVSCKASGYTFKEYGISWVRQAPGQGLEWMGWISAYSGHTYYAQKLQGRVTMTTDTSTSTAYMELRSLRSDDTAVYYCARGPHYDDWSGFIIWFDPWGQGTLVTVSSGSTSGSGKPGSGEGSTKGDIQMTQSPSSLSASVGDRVTITCRASQSISSYLNWYQQKPGKAPKLLIYAASSLQSGVPSRFSGSGSGTDFTLTISSLQPEDFATYYCQQSYRFPPTFGQGTKVEIK

SEQ ID NO: 15 - protein, 57 aa, synthetic construct
FVPVFLPAKPTTTPAPRPPTPAPTIASQPLSLRPEACRPAAGGAVHTRGLDFACDIY

SEQ ID NO: 16 - protein, 26 aa, synthetic construct
IWAPLAGTCGVLLLSLVITLYCNHRN

SEQ ID NO: 17 - protein, 47 aa, synthetic construct
RFSVVKRGRKKLLYIFKQPFMRPVQTTQEEDGCSCRFPEEEEGGCEL

SEQ ID NO: 18 - protein, 22 aa, synthetic construct
MLLLVTSLLLCELPHPAFLLIP

SEQ ID NO: 19 - protein, 26 aa, synthetic construct
RAKRSGSGEGRGSLLTCGDVEENPGP

SEQ ID NO: 20 - protein, 21 aa, synthetic construct
MALPVTALLLPLALLLHAARP

SEQ ID NO: 21 - protein, 5 aa, synthetic construct
DYGVS

SEQ ID NO: 22 - protein, 16 aa, synthetic construct
VIWGSETTYYNSALKS

SEQ ID NO: 23 - protein, 12 aa, synthetic construct
HYYYGGSYAMDY

SEQ ID NO: 24 - protein, 11 aa, synthetic construct
RASQDISKYLN

SEQ ID NO: 25 - protein, 7 aa, synthetic construct
HTSRLHS

SEQ ID NO: 26 - protein, 9 aa, synthetic construct
QQGNTLPYT

SEQ ID NO: 27 - protein, 5 aa, synthetic construct
EYGIS

SEQ ID NO: 28 - protein, 16 aa, synthetic construct
WISAYSGHTYYAQKLQ

SEQ ID NO: 29 - protein, 16 aa, synthetic construct
GPHYDDWSGFIIWFDP

SEQ ID NO: 30 - protein, 11 aa, synthetic construct
RASQSISSYLN

SEQ ID NO: 31 - protein, 7 aa, synthetic construct
AASSLQS

SEQ ID NO: 32 - protein, 9 aa, synthetic construct
QQSYRFPPT

SEQ ID NO: 33 - protein, 124 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARETDYSSGMGYGMDVWGQGTTVTVSS

SEQ ID NO: 34 - protein, 107 aa, synthetic construct
DIQMTQSPSSLSASVGDRVTITCRASQSINSYLNWYQQKPGKAPKLLIYAASSLQSGVPSRFSGSGSGTDFTLTISSLQPEDFATYYCQQSLADPFTFGGGTKVEIK

SEQ ID NO: 35 - protein, 127 aa, synthetic construct
QVQLQQWGAGLLKPSETLSLTCAVYGGSFSRYVWSWIRQPPGKGLEWIGEIDSSGKTNYNPSLKSRVTISVDTSKNQFSLKLSSVTAADTAVYYCARVRYDSSDSYYYSYDYGMDVWGQGTTVTVSS

SEQ ID NO: 36 - protein, 113 aa, synthetic construct
DIVLTQSPDSLAVSLGERATINCKSSQSVLYSSNNKNYLAWYQQKPGQPPKLLIYWASSRESGVPDRFSGSGSGTDFTLTISSLQAEDVAVYYCQQSYSFPWTFGGGTKVEIK

SEQ ID NO: 37 - protein, 127 aa, synthetic construct
QVQLQQWGAGLLKPSETLSLTCAVYGGSFSGYAWSWIRQPPGKGLEWIGEIDHRGFTNYNPSLKSRVTISVDTSKNQFSLKLSSVTAADTAVYYCARVRYDSSDSYYYSYDYGMDVWGQGTTVTVSS

SEQ ID NO: 38 - protein, 113 aa, synthetic construct; identical to SEQ ID NO: 36

SEQ ID NO: 39 - protein, 3096 aa, synthetic construct
ATGCTGCTGCTGGTGACATCTCTGCTGCTTTGCGAGCTGCCCCACCCTGCCTTCCTGCTTATCCCCGACATTCAGATGACCCAGACCACCAGCAGCCTGAGCGCCAGCTTAGGAGATAGAGTTACCATCAGCTGCAGAGCCAGCCAGGACATCAGCAAATACCTGAACTGGTATCAGCAGAAGCCCGACGGCACTGTGAAACTGCTTATTTACCACACCTCCAGACTGCACAGCGGCGTTCCCAGCAGATTCTCTGGCAGCGGATCTGGAACCGACTACAGCCTCACCATCTCCAACCTGGAGCAGGAGGACATCGCCACCTACTTCTGCCAGCAGGGCAACACACTGCCCTACACCTTCGGAGGAGGAACCAAGCTGGAGATCACCGGCTCCACCTCTGGATCCGGCAAGCCCGGATCTGGCGAGGGATCCACCAAGGGCGAGGTTAAGCTGCAGGAGAGCGGCCCTGGCCTGGTGGCTCCTAGCCAATCTTTATCTGTGACCTGCACTGTGTCCGGCGTTAGCCTGCCCGATTATGGCGTTTCCTGGATCAGACAGCCCCCCAGAAAGGGCCTGGAATGGCTGGGCGTTATCTGGGGCAGCGAGACCACATACTACAACAGCGCCCTGAAGAGCAGACTTACGATTATCAAGGACAACAGCAAGAGCCAGGTTTTCCTGAAGATGAACAGCCTGCAGACCGACGACACCGCCATCTACTACTGCGCTAAGCACTACTACTACGGCGGCAGCTACGCCATGGACTACTGGGGCCAGGGAACAAGCGTTACCGTTAGCAGCGCTGCTGCAATTGAAGTTATGTATCCTCCTCCTTACCTGGACAACGAGAAGAGCAACGGCACCATCATCCACGTTAAGGGCAAGCACCTGTGCCCCAGCCCTCTGTTCCCTGGACCTTCTAAGCCTTTCTGGGTTCTGGTGGTGGTCGGCGGCGTTTTAGCCTGTTACAGCCTTCTGGTGACTGTGGCCTTCATCATCTTTTGGGTTAGAAGCAAGAGAAGCAGACTGCTCCACAGCGACTACATGAACATGACCCCCAGACGGCCTGGCCCCACCAGAAAGCATTACCAGCCCTACGCTCCTCCCAGAGACTTCGCCGCCTACAGGAGCAGAGTTAAATTCAGCAGATCCGCCGATGCCCCCGCTTACCAACAGGGACAAAACCAGCTGTACAATGAGCTCAACCTGGGGAGAAGAGAAGAATACGACGTTCTGGATAAGAGAAGGGGCAGAGATCCCGAAATGGGGGGCAAGCCCAGACGCAAGAACCCTCAGGAGGGGCTTTACAACGAACTGCAGAAGGATAAGATGGCTGAGGCTTACTCGGAGATTGGGATGAAGGGGGAGAGAAGGCGGGGCAAGGGACACGATGGCTTATACCAGGGGCTGAGCACCGCCACCAAGGACACATACGACGCTCTTCATATGCAGGCTCTGCCCCCAAGAAGGGCTAAGAGATCTGGCTCTGGCGAGGGCAGAGGCAGCTTGCTTACATGTGGCGATGTGGAGGAGAACCCCGGGCCCATGGCTCTTCCTGTGACAGCTCTTCTGCTGCCCCTGGCCCTGCTTCTGCATGCTGCTAGACCTCAGGTTCAGCTTGTGCAGAGCGGAGCTGAAGTTAAGAAGCCTGGCGCCTCTGTGAAGGTTAGCTGCAAGGCCAGCGGCTACACATTCAAGGAATATGGCATCTCCTGGGTTAGGCAGGCTCCCGGCCAAGGCTTAGAATGGATGGGCTGGATCTCCGCCTACTCCGGCCACACCTACTACGCCCAGAAGCTTCAGGGCAGGGTTACCATGACCACCGACACCAGCACCTCTACCGCCTATATGGAGCTGAGGAGCCTGAGATCGGACGACACAGCTGTGTATTACTGCGCCAGAGGCCCCCACTACGACGACTGGTCTGGATTTATCATCTGGTTCGACCCCTGGGGGCAGGGCACCCTGGTCACAGTTTCTTCTGGCTCCACCAGCGGAAGCGGCAAGCCAGGCTCAGGCGAAGGATCTACAAAAGGCGACATCCAAATGACACAGAGCCCCAGCAGCTTGAGCGCCTCCGTTGGCGACAGAGTTACAATCACCTGCAGGGCCTCTCAGAGCATCAGCAGCTATTTGAATTGGTATCAACAGAAGCCAGGAAAGGCCCCTAAGCTGCTCATCTACGCTGCCAGCTCGCTCCAATCTGGCGTTCCTAGCAGATTTAGCGGCTCCGGCAGCGGCACAGACTTTACTCTTACCATTAGCTCCCTGCAGCCCGAGGACTTCGCTACCTACTATTGCCAGCAAAGCTACAGATTCCCTCCCACCTTTGGCCAGGGCACAAAGGTTGAGATCAAGGCAGCTGCTTTCGTGCCTGTGTTCCTGCCTGCTAAGCCCACCACCACTCCTGCTCCAAGACCTCCTACCCCCGCTCCTACAATCGCCAGCCAACCTCTGAGCCTGAGACCGGAGGCATGCAGACCTGCGGCAGGGGGAGCAGTTCACACAAGAGGCTTGGACTTCGCTTGCGACATCTACATCTGGGCCCCTCTGGCCGGCACATGCGGAGTTCTTCTTCTTAGCCTGGTGATCACCCTGTACTGCAACCACAGAAACAGATTCAGCGTTGTGAAGAGAGGCCGGAAGAAGCTGCTGTACATCTTCAAGCAGCCCTTCATGAGACCTGTGCAGACCACACAGGAGGAAGACGGCTGCAGCTGTAGATTCCCCGAGGAAGAGGAGGGCGGCTGTGAGCTGAGAGTTAAGTTCAGCAGGAGCGCCGACGCCCCTGCCTACCAGCAAGGACAGAATCAACTGTACAACGAGCTGAACCTGGGCAGACGGGAGGAATACGATGTGCTGGACAAGAGGAGAGGCAGAGACCCCGAGATGGGCGGCAAACCTAGAAGAAAGAACCCCCAGGAGGGCCTGTATAACGAGCTCCAGAAGGACAAGATGGCCGAGGCCTACAGCGAGATCGGCATGAAGGGCGAAAGAAGAAGAGGCAAGGGCCACGACGGCCTCTACCAGGGCTTAAGCACAGCTACAAAGGACACCTACGACGCCCTGCACATGCAGGCCCTGCCCCCTAGATGA